SEQUENCE LISTING (74 sequences)

SEQ ID NO: 1 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 2 - protein, 389 aa, synthetic construct
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 3 - protein, 419 aa, synthetic construct
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 4 - protein, 406 aa, synthetic construct
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 5 - protein, 451 aa, synthetic construct
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 6 - protein, 386 aa, synthetic construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 7 - protein, 386 aa, synthetic construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 8 - protein, 417 aa, synthetic construct
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 9 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 10 - protein, 386 aa, synthetic construct
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 11 - protein, 411 aa, synthetic construct
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 12 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 13 - protein, 408 aa, synthetic construct
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 14 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 15 - protein, 386 aa, synthetic construct
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 16 - protein, 386 aa, synthetic construct
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 17 - protein, 386 aa, synthetic construct
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 18 - protein, 386 aa, synthetic construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 19 - protein, 386 aa, synthetic construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 20 - protein, 386 aa, synthetic construct
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 21 - protein, 386 aa, synthetic construct
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 22 - protein, 386 aa, synthetic construct
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 23 - protein, 386 aa, synthetic construct
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 24 - protein, 386 aa, synthetic construct
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 25 - protein, 386 aa, synthetic construct
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 26 - protein, 386 aa, synthetic construct
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 27 - protein, 391 aa, synthetic construct
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 28 - protein, 386 aa, synthetic construct
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 29 - protein, 386 aa, synthetic construct
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 30 - protein, 386 aa, synthetic construct
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 31 - protein, 386 aa, synthetic construct
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 32 - protein, 385 aa, synthetic construct
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 33 - protein, 386 aa, synthetic construct
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 34 - protein, 386 aa, synthetic construct
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 35 - protein, 386 aa, synthetic construct
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 36 - protein, 385 aa, synthetic construct
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 37 - protein, 386 aa, synthetic construct
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 38 - protein, 386 aa, synthetic construct
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 39 - protein, 386 aa, synthetic construct
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 40 - protein, 386 aa, synthetic construct
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 41 - protein, 386 aa, synthetic construct
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 42 - protein, 386 aa, synthetic construct
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 43 - protein, 386 aa, synthetic construct
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 44 - protein, 386 aa, synthetic construct
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 45 - protein, 386 aa, synthetic construct
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 46 - protein, 381 aa, synthetic construct
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 47 - protein, 391 aa, synthetic construct
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 48 - protein, 383 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 49 - protein, 386 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 50 - protein, 392 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 51 - protein, 386 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 52 - protein, 395 aa, synthetic construct
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 53 - protein, 386 aa, synthetic construct
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 54 - protein, 386 aa, synthetic construct
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 55 - protein, 385 aa, synthetic construct
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 56 - protein, 386 aa, synthetic construct
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 57 - protein, 383 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 58 - protein, 386 aa, synthetic construct
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 59 - protein, 386 aa, synthetic construct
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 60 - protein, 2174 aa, synthetic construct
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 61 - protein, 386 aa, synthetic construct
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 62 - protein, 2104 aa, synthetic construct
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 63 - protein, 386 aa, synthetic construct
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 64 - protein, 420 aa, synthetic construct
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 65 - protein, 386 aa, synthetic construct
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 66 - protein, 386 aa, synthetic construct
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 67 - protein, 386 aa, synthetic construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 68 - protein, 386 aa, synthetic construct
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 69 - protein, 386 aa, synthetic construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 70 - protein, 399 aa, synthetic construct
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 71 - protein, 389 aa, synthetic construct
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 72 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 73 - protein, 389 aa, synthetic construct
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 74 - protein, 484 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSPNORMALDOTM

(19) United States
(12) Patent Application Publication
(10) Pub. No.: US 2025/0019396 A1
(43) Pub. Date: Jan. 16, 2025

(54) RECOMBINANT PROTEIN RECOVERY METHODS

(71) Applicant: Clara Foods Co., Daly City, CA (US)

(72) Inventors: Aniket KALE, Pleasanton, CA (US); Ranjan PATNAIK, Daly City, CA (US)

(73) Assignee: Clara Foods Co., Daly City, CA (US)

(21) Appl. No.: 18/903,982

(22) Filed: Oct. 1, 2024

Related U.S. Application Data
(63) Continuation of application No. PCT/US2023/065167, filed on Mar. 30, 2023.
(60) Provisional application No. 63/346,809, filed on May 27, 2022.
(60) Provisional application No. 63/326,278, filed on Apr. 1, 2022.

(51) Int. Cl. C07K 1/36; A23J 1/00; C12N 1/14; C12R 1/66

(52) U.S. Cl. CPC C07K 1/36; A23J 1/008; C12N 1/14; C12R 2001/66

(57) ABSTRACT

The present disclosure relates to methods for increasing recovery and purity of a secreted protein of interest, recovered proteins from the methods, and uses of the proteins.

### CROSS-REFERENCE TO RELATED APPLICATIONS

This application is a continuation of International Patent Application No. PCT/US2023/065167, filed Mar. 30, 2023, which claims the benefit of U.S. Provisional Application No. 63/326,278, filed Apr. 1, 2022, and U.S. Provisional Application No. 63/346,809, filed May 27, 2022; the contents of each of which is incorporated by reference herein in its entirety.

### SEQUENCE LISTING

The instant application contains a Sequence Listing which has been submitted electronically in XML format and is hereby incorporated by reference in its entirety. Said XML copy, created on Mar. 29, 2023, is named 49160752601.xml and is 97,017 bytes in size.

### BACKGROUND

The recombinant proteins can be produced by precision fermentation by expressing in multiple host systems such as bacteria, yeast, and fungi. Fungal systems have an inherent issue in that they express both a protein of interest and native fungal proteins. Thus, when using fungal expression systems, additional, complicated, time-consuming, and expensive steps are needed for recovering and purifying proteins of interest. There remains an unmet need for simplified recovery and purification of proteins of interest from fungal expression systems.

### SUMMARY

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

An aspect of the present disclosure is a method for increasing recovery and purity of a secreted protein of interest. The method comprising steps of: obtaining recombinant fungal cells capable of expressing a secreted protein of interest; culturing the recombinant fungal cells under conditions that promote expression and secretion of the recombinant protein of interest into a culturing medium; adding an acid to the culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; and recovering the precipitated protein of interest.

Another aspect of the present disclosure is a powdered composition comprising any herein-disclosed recovered protein of interest.

Yet another aspect of the present disclosure is a liquid composition comprising a solvent suitable for animal or human consumption and a powdered composition comprising any herein-disclosed recovered protein of interest.

In an aspect, the present disclosure provides a method for increasing recovery and purity of a secreted protein of interest. The method comprising steps of: obtaining recombinant fungal cells capable of expressing a secreted protein of interest; culturing the recombinant fungal cells under conditions that promote expression and secretion of the recombinant protein of interest into a culturing medium; centrifuging the culturing medium and excluding the recombinant fungal cells and other cellular components; microfiltering the centrifuged culturing medium to further remove any residual cell components prior to adding the acid and introducing ammonium sulfate; adding an acid to the microfiltered culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; and recovering the precipitated protein of interest.

In another aspect, the present disclosure provides a method for increasing recovery and purity of a secreted protein of interest. The method comprising steps of: obtaining recombinant fungal cells capable of expressing a secreted protein of interest; culturing the recombinant fungal cells under conditions that promote expression and secretion of the recombinant protein of interest into a culturing medium; centrifuging the culturing medium and excluding the recombinant fungal cells and other cellular components; microfiltering the centrifuged culturing medium to further remove any residual cell components prior to adding the acid and introducing ammonium sulfate; adding an acid to the microfiltered culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; recovering the precipitated protein of interest; solubilizing the precipitated secreted protein of interest with water to obtain a solubilized protein of interest; diafiltering and/or ultrafiltering the solubilized protein of interest; microfiltering the diafiltered and/or ultrafiltered protein of interested; and drying the further microfiltered protein of interest, thereby obtaining a dried protein product.

Additionally, any composition, food product, ingredient, use, or method disclosed herein is applicable to any herein-disclosed composition, food product, ingredient, use, or method. In other words, any aspect or embodiment described herein can be combined with any other aspect or embodiment as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A and FIG. 1B are flow charts showing illustrative steps of methods of the present disclosure.

FIG. 2 is a photograph showing centrifuge tubes containing pelleted protein. The proteins were precipitated with increasing pH from left to right (pH 3.25 to pH 5.5).

FIG. 3 is a SDS PAGE gel for proteins precipitated at various pH values.

FIG. 4 is a graph showing percentages of protein recovery for proteins precipitated at various pH values.

FIG. 5 is a graph showing percentages of protein that was not recovered, i.e., proteins remaining in the supernatant, for proteins precipitated at various pH values. The percentages shown on the Y-axis are in decimal; thus, “0.1” means “10%”. POI: protein of interest; supe: supernatant.

FIG. 6 is a graph showing the amount of protein (in grams/liter) recovered (in blue and bottom portion of each bar) and the amount of protein that was not recovered, i.e., proteins remaining in the supernatant (in orange and top portion of each bar), for proteins precipitated at various concentrations of ammonium sulfate.

FIG. 7 is a graph showing the percentage of protein recovered for proteins precipitated at various concentrations of ammonium sulfate, with pH at 4.5 throughout.

FIG. 8A is an SDS PAGE gel demonstrating the purification of the protein with certain process conditions; lanes 9 and 10 are duplicates of the supernatant and lanes 20 and 21 are the respective pellets resuspended in DI water to the initial volume. FIG. 8B includes chromatograms (top two images) for supernatant samples 9 and 10 of FIG. 8A and chromatogram (bottom two images) for precipitant samples 20 and 21.

FIG. 9 includes a flow chart of a first run of a recovery method of the present disclosure. Shown are chromatograms showing recovery and purity of protein at various stages in the method and also shown are amounts of protein of interest (POI) recovered at the various stages. MF: microfiltration; UF-DF: ultrafiltration-diafiltration.

FIG. 10 includes a flow chart of a second run of a recovery method of the present disclosure. Shown are chromatograms showing recovery and purity of protein at various stages in the method and also shown are amounts of protein of interest (POI) recovered at the various stages. MF: microfiltration; UF-DF: ultrafiltration-diafiltration.

FIG. 11 to FIG. 13 are photographs of compositions made from the recovered protein of interest of Examples 4 and 5. In FIG. 11, a dough is shown, in FIG. 12, a liquid composition is shown, and FIG. 13, a foam is shown.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure relates to methods for increasing recovery and purity of a secreted protein of interest, recovered proteins from the methods, and uses of the proteins.

### Illustrative Methods

An aspect of the present disclosure is a method for increasing recovery and purity of a secreted protein of interest. The method comprising steps of: obtaining recombinant fungal cells capable of expressing a secreted protein of interest; culturing the recombinant fungal cells under conditions that promote expression and secretion of the recombinant protein of interest into a culturing medium; adding an acid to the culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; and recovering the precipitated protein of interest.

In some embodiments, the method comprises a step of centrifuging the culturing medium and excluding the recombinant fungal cells and other cellular components after culturing the recombinant fungal cells. In some cases, once fermentation is complete, the culturing medium may be diluted, chilled, and clarified using a centrifuge. The solids arc disposed of and the centrate can be filtered through a 0.2 um filter in a TFF mode (hollow fiber/spiral wound) to remove any remaining cell debris. The filtrate may be stored at 8° C. for up to 72 hours at this point.

In various embodiments, the method comprises a step of microfiltering the centrifuged culturing medium to further remove any residual cell components prior to adding the acid and introducing ammonium sulfate.

In embodiments, the microfiltering comprises a filter capable of capturing fungal cells and other cellular components. In some cases, the filter is a 0.2 μm filter. In some cases, the permeate from the 0.2 μm filter is reduced in volume by a 10 kDa membrane, thereby concentrating the filtrate. The filtrate may be concentrated by this step 2-fold, 3-fold, 4-fold, 5-fold, 6-fold, 7-fold, 8-fold, 9-fold, or 10-fold.

In some embodiments, a precipitate is recovered by use of a centrifuge or a Sedicanter®. When centrifuging, extra care can be used when balancing the feed solids percentage, the feed rate, and the residence time in the bowl, thereby helping ensure that solids do not adhere to the bowl. Should solids adhere a water rinse can be performed through the machine and cyclone that is not allowed to go out the centrate. This rinse can help recover protein, since the POI is very soluble. Typical disk stack centrifuges have wash nozzles on the bowl and cyclone to help remove solids. The Sedicanter® from Flottweg can be used to recover the precipitate and may be more efficient than the centrifuge.

In some embodiments, the method comprises a step solubilizing the precipitated secreted protein of interest with water, e.g., DI water, to obtain a solubilized protein of interest. In some cases, a protein concentration target of 40-50 g/L may help achieve proper solubilization. In various cases, the pH can be increased to about 6 with sodium hydroxide.

In various embodiments, the method comprises a step diafiltering and/or ultrafiltering the solubilized protein of interest. In some cases, the diafiltration comprises a 10 kDa membrane. This step remove salts from the solubilized protein of interest. This filtration may be run in a tangential mode. The target protein is in the retentate. In some cases, a solubilized POI solution at pH 6.5±0.2 at ˜50 g/L protein concentration is diafiltered until the conductivity in the retentate is <900 uS/cm. The final dialyzed material will be golden yellow and clear and at about pH 6.9±0.2. Typically, this would mean diafiltering around 6-8 DVs.

In embodiments, the method comprises a step of further microfiltering the diafiltered and/or ultrafiltered protein of interested. Here, the retentate is sterile filtered using a 0.2 um MF filter. This helps ensure that the final product substantially lacks microbial contamination.

In some embodiments, the method comprises a step of drying the further microfiltered protein of interest, thereby obtaining a dried protein product.

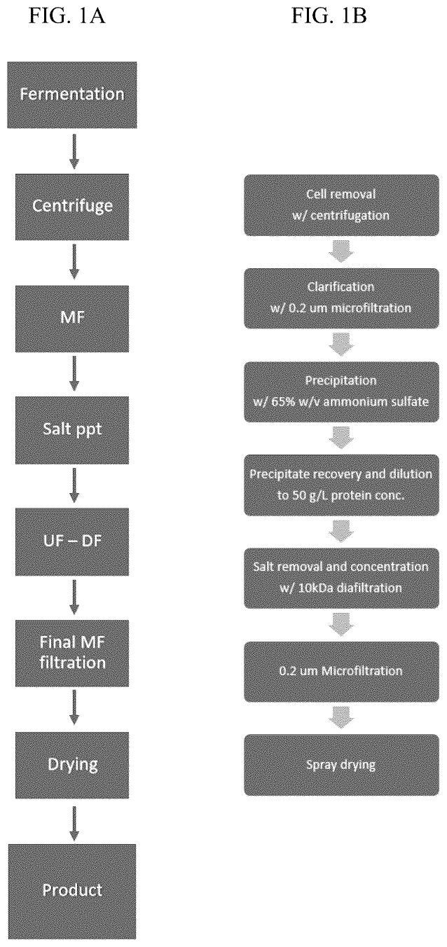

These methods and steps thereof are illustrated in FIG. 1A and FIG. 1B. In some embodiments, the method ends after the “Salt ppt” step and a precipitated protein may be recovered.

In an aspect, the present disclosure provides a method for increasing recovery and purity of a secreted protein of interest. The method comprising steps of: obtaining recombinant fungal cells capable of expressing a secreted protein of interest; culturing the recombinant fungal cells under conditions that promote expression and secretion of the recombinant protein of interest into a culturing medium; centrifuging the culturing medium and excluding the recombinant fungal cells and other cellular components; microfiltering the centrifuged culturing medium to further remove any residual cell components prior to adding the acid and introducing ammonium sulfate; adding an acid to the microfiltered culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; and recovering the precipitated protein of interest.

In another aspect, the present disclosure provides a method for increasing recovery and purity of a secreted protein of interest. The method comprising steps of: obtaining recombinant fungal cells capable of expressing a secreted protein of interest; culturing the recombinant fungal cells under conditions that promote expression and secretion of the recombinant protein of interest into a culturing medium; centrifuging the culturing medium and excluding the recombinant fungal cells and other cellular components; microfiltering the centrifuged culturing medium to further remove any residual cell components prior to adding the acid and introducing ammonium sulfate; adding an acid to the microfiltered culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; recovering the precipitated protein of interest; solubilizing the precipitated secreted protein of interest with water to obtain a solubilized protein of interest; diafiltering and/or ultrafiltering the solubilized protein of interest; microfiltering the diafiltered and/or ultrafiltered protein of interested; and drying the further microfiltered protein of interest, thereby obtaining a dried protein product.

In some embodiments, the method comprises a step of centrifuging the culturing medium and excluding the recombinant fungal cells and other cellular components after culturing the recombinant fungal cells.

In various embodiments, the method comprises a step of microfiltering the centrifuged culturing medium to further remove any residual cell components prior to adding the acid and introducing ammonium sulfate.

In embodiments, the microfiltering comprises a filter capable of capturing fungal cells and other cellular components. In some cases, the filter is a 0.2 μm filter.

In some embodiments, the method comprises a step solubilizing the precipitated secreted protein of interest with water, e.g., DI water, to obtain a solubilized protein of interest. In some cases, a protein concentration target of 40-50 g/L may help achieve proper solubilization. In various cases, the pH can be increased to about 6 with sodium hydroxide.

In various embodiments, the method comprises a step diafiltering and/or ultrafiltering the solubilized protein of interest.

In embodiments, the method comprises a step of further microfiltering the diafiltered and/or ultrafiltered protein of interested.

In some embodiments, the method comprises a step of drying the further microfiltered protein of interest, thereby obtaining a dried protein product.

In various embodiments, the pH is reduced to a pH below 5, below 4.75, below 4.5, below 4.25, below 4.0, below 3.75, below 3.5, or below 3.25.

In embodiments, the pH is reduced to a pH of about 5, about 4.75, about 4.5, about 4.25, about 4.0, about 3.75, about 3.5, or about 3.25.

In some embodiments, the acid is phosphoric acid, e.g., 85% v/v phosphoric acid.

In various embodiments, adding the acid occurs before introducing the ammonium sulfate.

In embodiments, adding the acid occurs after introducing the ammonium sulfate.

In some embodiments, adding the acid is contemporaneous with introducing the ammonium sulfate.

In various embodiments, the ammonium sulfate concentration is above 200 g/l, the ammonium sulfate concentration is above 300 g/l, or the ammonium sulfate concentration is above 400 g/l.

In embodiments, the ammonium sulfate concentration is about 200 g/l, the ammonium sulfate concentration is about 300 g/l, or the ammonium sulfate concentration at is about 400 g/l.

In various embodiments, the ammonium sulfate is provided as a concentrated solution. In some case, the concentrated solution comprises about 30% w/v ammonium sulfate, about 35% w/v ammonium sulfate, about 40% w/v ammonium sulfate, about 45% w/v ammonium sulfate, about 50% w/v ammonium sulfate, about 55% w/v ammonium sulfate, about 60% w/v ammonium sulfate, about 65% w/v ammonium sulfate, about 70% w/v ammonium sulfate, or about 75% w/v ammonium sulfate. In some cases the concentrated solution comprises about 65% w/v ammonium sulfate.

In some embodiments, the pH is about or below 4.75, is about or below 4.5, is about or below 4.25, is about or below 4.0, is about or below 3.75, is about or below 3.5, or is about or below 3.25 and wherein the ammonium sulfate concentration is about or above 200 g/l, the ammonium sulfate concentration is about or above 300 g/l, or the ammonium sulfate concentration about is or above 400 g/l.

In various embodiments, the pH is about or below 4.75 and the ammonium sulfate concentration is about or above 300 g/l.

In embodiments, the pH is about or below 4.75 and the ammonium sulfate concentration is about or above 400 g/l.

In some embodiments, the pH is about or below 4.5 and the ammonium sulfate concentration is about or above 300 g/l.

In various embodiments, the pH is about or below 4.5 and the ammonium sulfate concentration is about or above 400 g/l.

In embodiments, the pH is about or below 4.25 and the ammonium sulfate concentration is about or above 300 g/l.

In some embodiments, the pH is about or below 4.25 and the ammonium sulfate concentration is about or above 400 g/l.

In various embodiments, the pH is about or below 4.0 and the ammonium sulfate concentration is about or above 300 g/l.

In embodiments, the pH is about or below 4.0 and the ammonium sulfate concentration is about or above 400 g/l.

In some embodiments, the pH is about or below 3.75 and the ammonium sulfate concentration is about or above 300 g/l.

In various embodiments, the pH is about or below 3.75 and the ammonium sulfate concentration is about or above 400 g/l.

In embodiments, the pH is about or below 3.5 and the ammonium sulfate concentration is about or above 300 g/l.

In some embodiments, the pH is about or below 3.5 and the ammonium sulfate concentration is about or above 400 g/l.

In various embodiments, the pH is about or below 3.25 and the ammonium sulfate concentration is about or above 300 g/l.

In embodiments, the pH is about or below 3.25 and the ammonium sulfate concentration is about or above 400 g/l.

In various embodiments, the ammonium sulfate is added with moderate mixing to allow the salt to fully dissolve avoiding clumps and poor precipitation. A milky white precipitate will form, and hard agitation will cause foaming. Thus, moderate agitation and tank chilling (as compared to using an external heat exchanger and pump) is preferred.

Precipitation of the protein of interest may take a few hours to almost a day, e.g., 2 hours, 3 hours, 4 hours, 5 hours, 6 hours, 7 hours, 8 hours, 9 hours, 10 hours, 11 hours, 12 hours, 13 hours, 14 hours, 16 hours, 17 hours, 18 hours, 19 hours, or 20 hours.

In some cases, after the protein is recovered by decanting or aspirating the ammonium sulfate-protein solution (slurry) from the container in which precipitation occurred. Any residual precipitate that is adhered to the container may be removed from the container by washing, e.g., with water, or by scraping the container, e.g., with a sterile implement.

In some embodiments, the amount of recovered protein of interest is greater than the recovery that is obtained from a method that does not comprise reducing the pH of the culturing medium to about or below the pI of the protein of interest and does not comprise adding ammonium sulfate to the culturing medium to achieve an ammonium sulfate concentration above 200 g/l.

In various embodiments, recovery of the protein of interest is at least about 40% w/w, is at least about 45% w/w, is at least about 50% w/w, is at least about 55% w/w, or is at least about 60% w/w for the final dried product, wherein the recovery is the weight of the protein of interest recovered in the final product relative to the weight of the protein of interest prior to adding the acid and introducing ammonium sulfate.

In some embodiments, recovery of the protein of interest is at least about 40% w/w, is at least about 45% w/w, is at least about 50% w/w, is at least about 55% w/w, is at least about 60% w/w, is at least about 65% w/w, is at least about 70% w/w, is at least about 75% w/w, is at least about 80% w/w, is at least about 85% w/w, is at least about 90% w/w, for the final dried product, wherein the recovery is the weight of the protein of interest recovered in the final product relative to the sum of weight of the protein of recovered and the weight of the protein remaining in the supernatant following adding the acid and introducing ammonium sulfate.

In embodiments, the purity of recovered protein of interest is greater than the purity that is obtained from a method that does not comprise reducing the pH of the culturing medium to about or below the pI of the protein of interest and does not comprise adding ammonium sulfate to the culturing medium to achieve an ammonium sulfate concentration above 200 g/l.

In some embodiments, the purity is at least about 75% w/w, is at least about 80% w/w, is at least about 85% w/w is at least about 90% w/w, is at least about 95% w/w for the final dried product, wherein the purity is defined as the weight of the protein of interest relative to the total weight of solid product.

In various embodiments, the method does not comprise use of a purification resin and/or a purification column. This is noteworthy in that many protein purification methods rely on purification resins and/or purification columns.

In embodiments, the fungal cells are of the species selected from Agaricus bisporus; Agaricus spp.; Aspergillus awamori; Aspergillus fumigatus; Aspergillus nidulans; Aspergillus niger; Aspergillus oryzae; Aspergillus oryzae; Aspergillus spp.; Colletotrichum gloeosporiodes; Colletotrichum spp.; Endothia parasitica; Endothia spp.; Fusarium graminearum; Fusarium solani; Fusarium spp.; Komagatella pastoris.; Komagatella phaffi; Mucor miehei; Mucor pusillus; Mucor spp.; Myceliophthora spp.; Myceliophthora thermophila; Neurospora crassa; Neurospora spp.; Penicillium (Talaromyces) emersonii; Penicillium camemberti; Penicillium canescens; Penicillium chrysogenum; Penicillium funiculosum; Penicillium purpurogenum; Penicillium roqueforti; Penicillium spp.; Pichia angusta; Pichia pastoris; Pichia pastoris; Pichia Pastoris “MutS” strain (Graz University of Technology (CBS7435MutS) or Biogrammatics (BG11)); Pichia spp.; Pleurotus ostreatus; Pleurotus spp.; Rhizomucor miehei; Rhizomucor pusillus; Rhizomucor spp.; Rhizopus arrhizus; Rhizopus oligosporus; Rhizopus oryzae; Rhizopus spp.; Trichoderma altroviride; Trichoderma reesei; Trichoderma spp.; Trichoderma vireus; Yarrowia lipolytica; and Yarrowia spp. In some cases, the fungal cells are Aspergillus cells, e.g., of the species Aspergillus spp., Aspergillus awamori, Aspergillus fumigatus, Aspergillus nidulans, Aspergillus niger, or Aspergillus oryzae.

### Proteins of Interest

Any protein of interest that may be recombinantly expressed and secreted by a fungal cell may be used in methods of the present disclosure. Proteins that can be recombinantly expressed by a fungal cell but cannot normally be secreted by the fungal cell may still be recovered by methods of the present disclosure; in these cases, the protein of interest is modified (e.g., by genetic manipulation of its DNA code) to express a signal that permits its secretion from the fungal cells. Such secretion signals are well-known in the art and choice of signal (or DNA encoding the signal) can be selected based on the fungal cell used and/or the specific protein of interest.

Another aspect of the present disclosure is a powdered composition comprising any herein-disclosed recovered protein of interest.

Yet another aspect of the present disclosure is a liquid composition comprising a solvent suitable for animal or human consumption and a powdered composition comprising any herein-disclosed recovered protein of interest.

In some cases, the protein of interest is a food protein, e.g., which is used as nutritional, dietary, digestive, supplements, such as in food products and feed products. The food protein may be a plant protein or may be an animal protein.

The animal protein may be an egg white protein, e.g., selected from ovalbumin, ovomucoid, ovotransferrin, lysozyme, ovalbumin, ovomucoid, ovotransferrin, lysozyme, ovomucin, ovoglobulin G2, ovoglobulin G3, ovoinhibitor, ovoglycoprotein, flavoprotein, ovomacroglobulin, ovostatin, cystatin, avidin, ovalbumin related protein X, ovalbumin related protein Y, and any combination thereof.

In some cases, the egg white protein is an ovalbumin (OVA) that comprises the amino acid sequence of a chicken OVA, a goose OVA, a quail OVA, an ostrich OVA, or a duck OVA. The egg white protein may have at least 70%, 75%, 80%, 85%, 90%, 95%, 96%, 97%, 98%, or 99% sequence identity to one of SEQ ID NO: 1 to SEQ ID NO: 74.

An rOVA can be a non-naturally occurring variant of an OVA. Such variant can comprise one or more amino acid insertions, deletions, or substitutions relative to a native OVA sequence.

Such a variant can have at least 70%, 75%, 80%, 85%, 90%, 95%, 96%, 97%, 98%, or 99% sequence identity to SEQ ID NOs: 1-74. The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software, with BLAST being the preferable alignment algorithm. Those skilled in the art can determine appropriate parameters for measuring alignment, including any algorithms needed to achieve maximal alignment over the full-length of the sequences being compared.

Depending on the host organism used to express the rOVA, the rOVA can have a glycosylation, acetylation, or phosphorylation pattern different from wildtype OVA. For example, the rOVA herein may or may not be glycosylated, acetylated, or phosphorylated. An rOVA may have an avian, non-avian, microbial, non-microbial, mammalian, or non-mammalian glycosylation, acetylation, or phosphorylation pattern.

In some cases, rOVA may be deglycosylated (e.g., chemically, enzymatically, Endo-H, PNGase F, O-Glycosidase, Neuraminidase, β1-4 Galactosidase, β-N-acetylglucosaminidase), deacetylated (e.g., protein deacetylase, histone deacetylase, sirtuin), or dephosphorylated (e.g., acid phosphatase, lambda protein phosphatase, calf intestinal phosphatase, alkaline phosphatase). Deglycosylation, deacetylation or dephosphorylation may produce a protein that is more uniform or is capable of producing a composition with less variation.

The present disclosure contemplates modifying glycosylation of the recombinant OVA to alter or enhance one or more functional characteristics of the protein and/or its production. In some embodiments, the change in rOVA glycosylation can be due to the host cell glycosylating the rOVA. In some embodiments, rOVA has a glycosylation pattern that is not identical to a native ovalbumin (nOVA), such as a nOVA from chicken egg. In some embodiments, rOVA is treated with a deglycosylating enzyme before it is used as an ingredient in an rOVA composition, or when rOVA is present in a composition. In some embodiments, the glycosylation of rOVA is modified or removed by expressing one or more enzymes in a host cell and exposing rOVA to the one or more enzymes. In some embodiments, rOVA and the one or more enzymes for modification or removal of glycosylation are co-expressed in the same host cell.

Native ovalbumin (nOVA), such as isolated from a chicken or another avian egg, has a highly complex branched form of glycosylation. The glycosylation pattern comprises N-linked glycan structures such as N-acetylglucosamine units, galactose and N-linked mannose units. In some cases, the rOVA for use in a herein disclosed consumable composition and produced using the methods described herein has a glycosylation pattern which is different from the glycosylation pattern of nOVA. For example, when rOVA is produced in a Pichia sp., the protein may be glycosylated differently from the nOVA and lack galactose units in the N-linked glycosylation. The glycosylation patterns of rOVA produced by P. pastoris have a complex branched glycosylation pattern. In some embodiments of the compositions and methods disclosed herein, rOVA is treated such that the glycosylation pattern is modified from that of nOVA and also modified as compared to rOVA produced by a Pichia sp. without such treatment. In some cases, the rOVA lacks glycosylation.

The molecular weight or rOVA may be different as compared to nOVA. The molecular weight of the protein may be less than the molecular weight of nOVA or less than rOVA produced by the host cell where the glycosylation of rOVA is not modified. In embodiments, the molecular weight of an rOVA may be between 40 kDa and 55 kDa. In some cases, an rOVA with modified glycosylation has a different molecular weight, such as compared to a native OVA (as produced by an avian host species) or as compared to a host cell that glycosylates the rOVA, such as where the rOVA includes N-linked mannosylation. In some cases, the molecular weight of rOVA is greater than the molecular weight of the rOVA that is completely devoid of post-translational modifications. or an rOVA that lacks all forms of N-linked glycosylation.

The compositions and methods provided herein contain fermentation-derived ovalbumin, produced through recombinant technology, i.e., a recombinant ovalbumin (rOVA). The compositions and methods for making compositions comprising rOVA can increase the protein content of a consumable or food ingredient, and also provide functional features for use in the preparation of food ingredients and consumable food products for animal and human ingestion.

In some embodiments, the rOVA provides one or more functional characteristics such as of gelling, foaming, whipping, fluffing, binding, springiness, aeration, coating, film forming, emulsification, browning, thickening, texturizing, humectant, clarification, and cohesiveness. The rOVA with such feature(s) can be a food ingredient that provides for production of an egg-less or animal-free food ingredient or food product.

As used herein “native” in the context of native egg white, native egg protein, native ovalbumin and native egg, refers to the egg white, egg protein, ovalbumin or whole egg, respectively, produced by an animal or collected from an animal, in particular an egg-laying animal such as a bird. The rOVA and compositions containing rOVA can be used in food ingredients and food products, such that the ingredient or product does not contain any native egg white, native egg protein, native ovalbumin or native egg. In some cases, the ingredients or food products made using rOVA do not include any egg-white proteins other than rOVA. The rOVA and compositions containing rOVA can be used in food ingredients and food products, such that the ingredient or product does not contain any animal products.

In some embodiments, the rOVA can (alone or with other ingredients) substitute for the use of whole egg or egg white in the production of a food product. In some embodiments, the feature(s) provided by the rOVA is substantially the same or better than the same characteristic provided by a native egg white or native egg. For example, the rOVA and compositions containing rOVA can have gelling, foaming, whipping, fluffing, binding, springiness, aeration, coating, film forming, emulsification, browning, thickening, texturizing, preserving moisture (humectant), clarification, and cohesiveness, improved color, such as a whiter color, as compared to native egg white or native whole egg and compositions made with native egg white.

### Food Ingredients and Food Products With rOVA

Food ingredients and food products disclosed herein include compositions that comprise, consists essentially of, or consist of rOVA, where rOVA provides at least one functional feature to the composition, food ingredient, or food product. In some cases, at least one functional feature provided by the rOVA is comparable or substantially similar to a native egg or egg white or native OVA (nOVA). For instance, it may provide any one of gelling, foaming, whipping, fluffing, binding, springiness, aeration, coating, film forming, emulsification, browning, thickening, texturizing, preserving moisture (humectant), clarification, and cohesiveness comparable to a whole egg, egg-white or nOVA composition. In some embodiments, the at least one functional feature is provided by or provided substantially by the inclusion of rOVA in the food ingredient or food product, for example, in the absence of any other whole egg proteins or egg white proteins.

Such compositions can include rOVA in an amount between 0.1% and 25% on a weight/weight (w/w) or weight/volume (w/v) basis. rOVA may be present at or at least at 0.1%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% on a weight/weight (w/w) or weight/volume (w/v) basis. These concentrations can be based on the dry weight of the composition. Additionally, or alternatively, the concentration of rOVA in such compositions is at most 30%, 20%, 15%, 10%, 5%, 4%, 3%, 2% or 1% on a w/w or w/v basis. In some embodiments, the rOVA in the food ingredient or food product can be at a concentration range of 0.1%-20%, 1%-20%, 0.1%-10%, 1%-10%, 0.1%-5%, 1%-5%, 2-10%, 4-8%, 4-10%, 4-12%, 0.1%-2%, 1%-2% or 0.1-1%.

Provided herein are consumable food compositions and methods of making such compositions where rOVA provides at least one feature of whole egg or egg-whites to a consumable food composition. In some embodiments, rOVA is added to a consumable food composition to increase the protein content, such as for added nutrition. In some embodiments, rOVA is present in the consumable food composition between about 1% and about 40% on a weight per total weight (w/w) and/or weight per total volume (w/v) of composition basis. For example, in a composition of 100 ml, rOVA is present at 30 g and the rOVA is thus at a 30% concentration (w/v) or for example, in a composition of 100 g, rOVA is present at 30 g and the rOVA is thus at a 30% concentration (w/w). In some embodiments, the concentration of rOVA is or is about 0.5%, 1%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% on a w/w and/or w/v of composition basis. In some embodiments, the rOVA is present at a concentration of or of about 0.5-1%, 1-5%, 2-8%, 4-8%, 2-12%, 4-12%, 5-10%, 10-15%, 15-20%, 20-25%, 25-30% or rOVA is present concentration greater than 1%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% w/w and/or w/v.

A consumable product can include one or more other proteins, such as a non-OVA protein or a non-recombinant protein. The rOVA can increase amount of protein content in a consumable product, and/or provide one or more egg-white like features. For example, the consumable composition can include a whey protein, a pea protein, a soy protein, an almond protein, an oat protein, a flax seed protein, a vegetable protein, or an egg-white protein. The consumable protein may include an extruded plant protein or a non-extruded plant protein. In some cases, the one or more other proteins can comprise OVA having an amino acid sequence naturally found in a bird or a reptile.

In some embodiments, the compositions and methods for making compositions have an egg-white like property and increase the protein content in the composition. In some embodiments, the compositions and methods for making compositions with an egg-white like property increase the protein content, while not adversely affecting the stability, or one or more sensory qualities of the composition.

In some embodiments, the consumable food compositions and methods for making consumable food compositions comprise rOVA and the addition of rOVA generates an egg-white like composition. The consumable food composition may be a finished product or an ingredient for making a finished product, e.g., a liquid or a powdered rOVA composition.

rOVA protein may be used on its own or in combination with other components to form a composition. In some embodiments, rOVA is used as an ingredient to form a composition and the rOVA ingredient (or rOVA starting composition to be added) may contain about or at least about 10%, 20%, 30%, 40%, 50%, 55%, 60%, 65%, 70%, 75%, 80%, 85%, 90%, 95%, 96%, 97%, 98%, or 99% rOVA by weight per total weight (w/w) and/or weight per total volume (w/v). In some cases, a composition described herein may contain up to about 10%, 20%, 30%, 40%, 50%, 55%, 60%, 65%, 70%, 75%, 80%, 85%, 90%, 95%, 96%, 97%, 98%, or 99% rOVA by w/w or w/v. In some embodiments, about or at least about 10%, 20%, 30%, 40%, 50%, 55%, 60%, 65%, 70%, 75%, 80%, 85%, 90%, 95%, 96%, 97%, 98%, or 99% of the protein in a composition is rOVA by weight per total weight (w/w) and/or weight per total volume (w/v). In some cases, up to or about 10%, 20%, 30%, 40%, 50%, 55%, 60%, 65%, 70%, 75%, 80%, 85%, 90%, 95%, 96%, 97%, 98%, or 99% of the protein in a composition is rOVA by w/w or w/v.

In some embodiments, a composition described herein contains total protein at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, or 75 g total protein per 100 mL liquid (e.g., water). In some cases, a composition described herein contains total protein at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, 75, 80, 85, 90, 95, or 100 g total protein per 100 g composition (e.g., powder).

In some embodiments, a composition described herein contains rOVA at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, or 75 g per 100 mL liquid (e.g., water). In some cases, a composition described herein contains rOVA at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, 75, 80, 85, 90, 95, or 100 g total protein per 100 g composition (e.g., powder)

In some embodiments, a composition described herein contains total protein at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5 g total protein per 100 mL liquid (e.g., water). In some cases, a composition described herein contains total protein at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5 g total protein per 100 g composition (e.g., powder).

In some embodiments, a composition described herein contains rOVA at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5 g per 100 mL liquid (e.g., water). In some cases, a composition described herein contains rOVA at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5 g per 100 g composition (e.g., powder).

In some embodiments, the rOVA consumable composition is a liquid composition. In such cases, the concentration of rOVA in the liquid composition may be between 0.1% to 90%. The concentration of rOVA in the liquid composition may be at least 0.1%. The concentration of rOVA in the liquid composition may be at most 90%. The concentration of rOVA in the liquid composition may be from 0.1% to 1%, 0.1% to 5%, 0.1% to 10%, 0.1% to 15%, 0.1% to 20%, 0.1% to 25%, 0.1% to 30%, 0.1% to 35%, 0.1% to 40%, 1% to 5%, 1% to 10%, 1% to 15%, 1% to 20%, 1% to 25%, 1% to 30%, 1% to 35%, 1% to 40%, 5% to 10%, 5% to 15%, 5% to 20%, 5% to 25%, 5% to 30%, 5% to 35%, 5% to 40%, 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 20% to 40%, 25% to 30%, 25% to 35%, 25% to 40%, 30% to 35%, 30% to 40%, 35% to 40%, 40% to 45%, 45% to 50%, 50% to 55%, 55% to 60%, 60% to 65%, 65% to 70%, 70% to 75%, 75% to 80%, 80% to 85%, 85% to 90%, or 90% to 95% in weight per total volume (w/v). The concentration of rOVA in the liquid composition may be about 0.1%, 1%, 5%, 10%, 15%, 20%, 25%, 30%, 35%, 40%, 45%, 50%, 55%, 60%, 65%, 70%, 75%, 80%, 85%, 90%, or 95% w/v. The concentration of rOVA in the liquid composition may be at least 0.1%, 1%, 5%, 10%, 15%, 20%, 25%, 30%, 35%, 40%, 45%, 50%, 55%, 60%, 65%, 70%, 75%, 80%, 85%, 90%, or 95% w/v. The concentration of rOVA in the liquid composition may be at most 1%, 5%, 10%, 15%, 20%, 25%, 30%, 35% 40%, 45%, 50%, 55%, 60%, 65%, 70%, 75%, 80%, 85%, 90%, or 95% w/v. In some embodiments, rOVA is the sole protein in the liquid composition. In other embodiments, a liquid composition comprises proteins other than rOVA.

In some embodiments, the rOVA consumable composition is a solid composition. In such cases, the concentration of rOVA in the solid composition may be between 0.1% to 70%. The concentration of rOVA in the solid composition may be at least 0.1%. The concentration of rOVA in the solid composition may be at most 70%. The concentration of rOVA in the solid composition may be 0.1% to 1%, 0.1% to 10%, 0.1% to 20%, 0.1% to 30%, 0.1% to 40%, 0.1% to 50%, 0.1% to 60%, 0.1% to 70%, 1% to 10%, 1% to 20%, 1% to 30%, 1% to 40%, 1% to 50%, 1% to 60%, 1% to 70%, 10% to 20%, 10% to 30%, 10% to 40%, 10% to 50%, 10% to 60%, 10% to 70%, 20% to 30%, 20% to 40%, 20% to 50%, 20% to 60%, 20% to 70%, 30% to 40%, 30% to 50%, 30% to 60%, 30% to 70%, 40% to 50%, 40% to 60%, 40% to 70%, 50% to 60%, 50% to 70%, or 60% to 70% weight per total weight (w/w) and/or weight per total volume (w/v). The concentration of rOVA in the solid composition may be 0.1%, 1%, 10%, 20%, 30%, 40%, 50%, 60%, or 70% w/w or w/v. The concentration of rOVA in the solid composition may be at least 0.1%, 1%, 10%, 20%, 30%, 40%, 50% or 60% w/w or w/v. The concentration of rOVA in the solid composition may be at most 1%, 10%, 20%, 30%, 40%, 50%, 60%, or 70% w/w or w/v.

In some embodiments, the rOVA consumable composition is a powdered composition. In such cases, the concentration of rOVA in the powder composition may be between 15% to 99% weight per total weight (w/w) and/or weight per total volume (w/v). The concentration of rOVA in the powder composition may be at least 15% w/w or w/v. In embodiments, the concentration of rOVA in the powder composition may be at most 99% w/w or w/v. The concentration of rOVA in the powder composition may be 15% to 30%, 15% to 45%, 15% to 60%, 15% to 75%, 15% to 80%, 15% to 85%, 15% to 90%, 15% to 95%, 15% to 99%, 30% to 45%, 30% to 60%, 30% to 75%, 30% to 80%, 30% to 85%, 30% to 90%, 30% to 95%, 30% to 99%, 45% to 60%, 45% to 75%, 45% to 80%, 45% to 85%, 45% to 90%, 45% to 95%, 45% to 99%, 60% to 75%, 60% to 80%, 60% to 85%, 60% to 90%, 60% to 95%, 60% to 99%, 75% to 80%, 75% to 85%, 75% to 90%, 75% to 95%, 75% to 99%, 80% to 85%, 80% to 90%, 80% to 95%, 80% to 99%, 85% to 90%, 85% to 95%, 85% to 99%, 90% to 95%, 90% to 99%, or 95% to 99% w/w or w/v. The concentration of rOVA in the powder composition may be about 15%, 30%, 45%, 60%, 75%, 80%, 85%, 90%, 95%, or 99% w/w or w/v. The concentration of rOVA in the powder composition may be at least 15%, 30%, 45%, 60%, 75%, 80%, 85%, 90% or 95% w/w or w/v. The concentration of rOVA in the powder composition may be at most 30%, 45%, 60%, 75%, 80%, 85%, 90%, 95%, or 99% w/w or w/v. In some embodiments, rOVA is the sole protein in the powder composition. In other embodiments, a powder composition comprises proteins other than rOVA.

In some cases, a powder composition may be a concentrate which comprises at least 70% rOVA w/w. In some cases, a powder composition may be a concentrate which comprises at least 80% rOVA w/w. In some cases, a powder composition may be an isolate which comprises at least 90% rOVA w/w. In some cases, a powder composition may be an isolate which comprises at least 95% rOVA w/w.

In some embodiments, the rOVA consumable composition is a concentrated liquid composition. In such cases, the concentration of rOVA in the concentrated liquid composition may be between 10% to 60% weight per total weight (w/w) and/or weight per total volume (w/v). The concentration of rOVA in the concentrated liquid may be at least 10% w/w or w/v. The concentration of rOVA in the concentrated liquid may be at most 60% w/w or w/v. The concentration of rOVA in the concentrated liquid may be 10% to 20%, 10% to 30%, 10% to 40%, 10% to 50%, 10% to 60%, 20% to 30%, 20% to 40%, 20% to 50%, 20% to 60%, 30% to 40%, 30% to 50%, 30% to 60%, 40% to 50%, 40% to 60%, or 50% to 60% w/w or w/v. The concentration of rOVA in the concentrated liquid may be about 10%, 20%, 30%, 40%, 50%, or 60% w/w or w/v. The concentration of rOVA in the concentrated liquid may be at least 10%, 20%, 30%, 40% or 50% w/w or w/v. The concentration of rOVA in the concentrated liquid may be at most 20%, 30%, 40%, 50%, or 60% w/w or w/v. The liquid may include any consumable solvent, e.g., water, dairy, oil, or other cooking base.

In some embodiments, the rOVA consumable composition is a prepared food for example, as a baked good, a salad dressing, an egg-like dish (such as an egg-patty or scramble), a dessert or dairy-like product or a meat-analog (such as a vegan meat patty, sausage or hot dog). Such compositions can include rOVA in an amount between 0.1% and 20% on a weight/weight (w/w) or weight/volume (w/v) basis. rOVA may be present at or at least at 0.1%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20% on a weight/weight (w/w) or weight/volume (w/v) basis. Additionally, or alternatively, the concentration of rOVA in such compositions is at most 30%, 20%, 15%, 10%, 5%, 4%, 3%, 2% or 1% on a w/w or w/v basis. In some embodiments, the rOVA in the food ingredient or food product can be at a concentration range of 0.1%-20%, 1%-20%, 0.1%-10%, 1%-10%, 0.1%-5%, 1%-5%, 0.1%-2%, 1%-2% or 0.1-1%.

### Features and Characteristics of rOVA Compositions and Food Ingredients and Food Products Containing rOVA

The rOVA containing compositions herein can provide one or more functional features to food ingredients and food products. In some embodiments, the rOVA provides a nutritional feature such as protein content, protein fortification and amino acid content to a food ingredient or food product. The nutritional feature provided by rOVA in the composition may be comparable or substantially similar to an egg, egg white or native OVA (nOVA). The nutritional feature provided by rOVA in the composition may be better than that provided by a native whole egg or native egg white. In some cases, rOVA provides the one or more functional features of egg-white in absence of any other egg-white proteins.

rOVA compositions disclosed herein can provide foaming and foam capacity to a composition. For example, rOVA can be used for forming a foam to use in baked products, such as cakes, for meringues and other foods where rOVA can replace egg white to provide foam capacity. In some cases, rOVA provides foaming and foam capacity of egg-white in absence of any other egg-white proteins.

A composition comprising rOVA may have a foam height greater than a foam height of an egg white or a composition comprising nOVA. In some cases, a composition comprising rOVA may have a foam height of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. In some cases, a composition comprising rOVA may have a foam height of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. Substitute egg whites may include products such as aquafaba, chia seeds, flax seeds, starches; apple sauce, banana puree; condensed milk, etc. which are commonly used as egg white substitutes.

A composition comprising rOVA may have a foam stability greater than a foam stability of an egg white, nOVA compositions or a substitute egg white. In some cases, a composition comprising rOVA may have a foam stability of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white or a substitute egg white. In some cases, a composition comprising rOVA may have a foam stability of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white. Foam stability may be calculated by measuring drainage of a foamed solution. The drainage may be measured in 10-minute increments for 30 minutes to gather data for foam stability. The drained volume after 30 minutes may be compared to the initial liquid volume (5 mL) for instance, foam Stability (%): (Initial volume−drained volume)/initial volume*100.

A composition comprising rOVA may have a foam capacity greater than a foam capacity of an egg white, nOVA compositions or a substitute egg white. In some cases, a composition comprising rOVA may have a foam capacity of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA or a substitute egg white. In some cases, a composition comprising rOVA may have a foam capacity of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. Foam capacity may be determined by measuring the initial volume of foam following the whipping and compare against the initial volume of 5 mL. Foam Capacity (%)=(volume of foam/initial volume)*100.

A liquid composition may foam faster than a composition comprising egg whites, nOVA or a substitute egg white. In some cases, an rOVA composition foams at least 1%, 5%, 10%, 15%, 20%, 25%, 30%, 35%, 40%, 45%, 50%, 55%, 60%, 65%, 70%, 75%, 80%, 85%, 90%, 95%, 100%, faster than an egg white, nOVA or substitute egg-white composition. In some cases, an rOVA composition foams up to 1%, 5%, 10%, 15%, 20%, 25%, 30%, 35%, 40%, 45%, 50%, 55%, 60%, 65%, 70%, 75%, 80%, 85%, 90%, 95%, 100% faster than an egg white, nOVA or substitute egg-white composition.

A composition comprising rOVA may have a gel strength greater than a gel strength of an egg white, nOVA composition or an egg white substitutes. In some cases, the rOVA composition may have a gel strength within the range from 100 g to 1500 g, from 500 g to 1500 g, or from 700 g to 1500 g. In some cases, an rOVA composition has a gel strength of about or at least 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, an rOVA composition has a gel strength of up to 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, an rOVA composition has a gel strength of about or at least 50%, 55%, 60%, 65%, 70%, 75%, 80%, 85%, 90%, 95%, 100% relative to an egg white, nOVA or egg white substitutes. In some cases, an rOVA composition has a gel strength of up to 50%, 55%, 60%, 65%, 70%, 75%, 80%, 85%, 90%, 95%, 100% relative to an egg white, nOVA or egg white substitutes.

rOVA compositions disclosed herein can provide structure, texture or a combination of structure and texture. In some embodiments, rOVA is added to a food ingredient or food product for baking and the rOVA provides structure, texture or a combination of structure and texture to the baked product. rOVA can be used in such baked products in place of native egg white, native egg or native egg protein. The addition of rOVA to baked products can also provide protein fortification to improve the nutritional content. In some embodiments, rOVA is used in a baked product in an amount between 0.1% and 25% on a weight/weight or weight/volume basis. In some embodiments, rOVA is used in a baked product in an amount between 0.1% and 5%. In some cases, rOVA provides the structure and/or texture of egg-white in absence of any other egg-white proteins.

rOVA compositions disclosed herein can be compatible with gluten formation, such that the rOVA can be used where gluten formation provides structure, texture and/or form to a food ingredient or food product.

Exemplary baked products in which rOVA can be used as an ingredient include, but are not limited to cake, cookie, bread, bagel, biscuits, muffin, cupcake, scone, pancake, macaroon, choux pastry, meringue, and soufflé. For example, rOVA can be used as an ingredient to make cakes such as pound cake, sponge cake, yellow cake, or angel food cake, where such cakes do not contain any native egg white, native whole egg or native egg protein. Along with rOVA, baked products may contain additional ingredients such as flour, sweetening agents, gum, hydrocolloids, starches, fibers, flavorings (such as flavoring extracts) and other protein sources. In some embodiments, a baked product may include rOVA and at least one fat or oil, at least one grain starch, and optionally at least one sweetener. Grain starch for use in such compositions include flours such as wheat flour, rice flour, corn flour, millet flour, spelt flour, and oat flour, and starches such as from corn, potato, sorghum, and arrowroot. Oil and fat for use in such compositions include plant-derived oils and fats, such as olive oil, corn oil, avocado oil, nut oils (e.g., almond, walnut and peanut) and safflower oil. rOVA may provide such baked goods with at least one characteristic of an egg white such as binding, springiness, aeration, browning, texturizing, humectant, and cohesiveness of the baked product. In some cases, the baked product does not comprise any natural egg white or natural egg, and/or does not include any other egg white derived proteins except rOVA. In some cases, rOVA is provided to the baked composition as an ingredient, such as starting with a concentrate, isolate or powder form of rOVA. In some cases, the rOVA provided as an ingredient for baked products is at a pH range between about 3.5 and 7.0. In some cases, a sweetener is included in the baked product such as a sugar, syrup, honey or sugar-substitute.

rOVA compositions disclosed herein can also be used to prepare egg-less food products, such as food products made where native whole egg or native egg white is a primary or featured ingredient such as scramble, omelet, patty, soufflé, quiche and frittata. In some embodiments, rOVA provides one or more functional features to the preparation including foaming, coagulation, binding, structure, texture, film-formation, nutritional profile, absence of cholesterol (i.e., cholesterol free) and protein fortification. Such egg-less preparations can be vegan, vegetarian, halal, or kosher, or a combination thereof. An egg-less preparation (also referred to as an egg-white substitute) may include rOVA and at least one fat or oil, a polysaccharide or polysaccharide-containing ingredient, and a starch. In some cases, the egg-less preparation may also include a flavoring agent (such as to provide a salty, sulfur-like or umami flavor), and/or a coloring agent (for example to provide yellow-like or off-white color to the baked product). In some cases, the inclusion or rOVA in the egg-less preparation provides a characteristic of natural (native) egg white such as hardness, adhesiveness, fracturability, cohesiveness, gumminess and chewiness when the composition is heated or cooked. Exemplary polysaccharide or polysaccharide-containing ingredients for such compositions include gellan gum, sodium alginate, and psyllium. Oil and fat for use in such compositions include plant-derived oils and fats, such as olive oil, corn oil, avocado oil, and safflower oil.

rOVA compositions disclosed herein can be used for a processed meat product or meat-like product, or for fish-like or shell-fish-like products. In such products, rOVA can provide one or more functional characteristics such as protein content and protein supplementations as well as binding, texturizing properties. Exemplary meat and meat-like products include burger, patty, sausage, hot dog, sliced deli meat, jerky, bacon, nugget and ground meat-like mixtures. Meat-like products can resemble beef, pork, chicken, lamb and other edible and consumed meats for humans and for other animals. Fish-like and shell-fish like products can resemble, for example, fish cakes, crab cakes, shrimp, shrimp balls, fish sticks, seafood meat, crab meat, fish fillets and clam strips. In some embodiments, rOVA is present in an amount between about 0.1% and 30% w/w/or w/v in the meat or meat-like product. In some embodiments, rOVA is used for a meat-like product (also referred to as a meat-analog and includes at least one fat or oil; and a plant-derived protein. Oil and fat for use in such compositions include plant-derived oils and fats, such as olive oil, corn oil, avocado oil, and safflower oil. Plant-derived proteins for use in meat analogs include soy protein, nut proteins, pea protein, lentil and other pulse proteins and whey proteins. In some cases, such plant protein is extruded, in other cases, such plant protein is non-extruded protein. In some cases, a meat analog includes rOVA at about 2% to 15% (w/w). In some cases, for meat analog compositions, rOVA acts as a binding agent, a gelling agent or a combination of a binding and gelling agent for such compositions.

rOVA compositions disclosed herein can be employed in coatings for food products. For example, rOVA can provide binding or adhesion characteristics to adhere batter or breading to another food ingredient. rOVA can be used as an “egg-less egg wash” where the rOVA protein provides appearance, color and texture when coated onto other food ingredients or food products, such as baked products. In one example, the “egg-less egg wash” may be used to coat a baked good such that the baked good adheres to a coating (e.g., seed, salt, spice, and herb). The addition of rOVA as a coating to a food product can provide a crunchy texture or increase the hardness, for example, of the exterior of a food product such as when the product is cooked, baked or fried.

rOVA compositions disclosed herein include sauces and dressings, such as an eggless mayonnaise, commercial mayonnaise substitutes, gravy, sandwich spread, salad dressing or food sauce. Inclusion of rOVA in a sauce or dressing, and the like, can provide one or more characteristics such as binding, emulsifying, odor neutrality, and mouthfeel. In some embodiments rOVA is present in such sauces and dressing in an amount between 0.1% and 3% or between about 3% and about 5% w/w/or w/v. In some cases, the amount of rOVA in a sauce or dressing may be substantially similar to the amount of whole egg, egg-white or nOVA used in a commercially available or commonly used recipe. Exemplary sauces and dressing include mayonnaise, commercial mayonnaise substitutes, alfredo sauce, and hollandaise sauce. In some embodiments, the rOVA-containing sauce or dressing does not contain whole egg, egg white, or any other protein extracted from egg. In some cases, the sauce, dressing or other emulsified product made with rOVA includes at least one fat or oil and water. Exemplary fats and oils for such compositions include corn oil, safflower oil, nut oils, and avocado oil.

rOVA compositions can be used to prepare confectionaries such as eggless, animal-free, vegetarian and vegan confectionaries. rOVA can provide one or more functional features to the confectionary including odor neutrality, flavor, mouthfeel, texture, gelling, cohesiveness, foaming, frothiness, nutritional value and protein fortification. In some embodiments, the prepared confectionary containing rOVA does not contain any native egg protein or native egg white. rOVA in such confectionaries can provide a firm or chewy texture. In some embodiments, rOVA is present between about 0.1% and 15% in a confectionary. Exemplary confectionaries include a gummy, a taffy, a divinity candy, meringue, marshmallow, and a nougat. In some embodiments, a confectionary includes rOVA, at least one sweetener and optionally a consumable liquid. Exemplary sweeteners include sugar, honey, sugar-substitutes and plant-derived syrups. In some cases, the rOVA is provided as an ingredient for making confectionaries at a pH between about 3.5 and about 7. In some cases, the rOVA is present in the confectionary composition at about 2% to about 15% (w/v). In some embodiments, the confectionary is a food product such as a meringue, a whipped dessert, or a whipped topping. In some embodiments, rOVA in the confectionary provides foaming, whipping, fluffing or aeration to the food product, and/or provides gelation. In some cases, the confectionary is a liquid, such as a foamed drink. In some cases, the liquid may include a consumable alcohol (such as in a sweetened cocktail or after-dinner drink).

rOVA compositions herein can be used in dairy products, dairy-like products or dairy containing products. For example, rOVA can be used in preparations of beverages such as a smoothie, milkshake, “egg-nog”, and coffee beverage. In some embodiments, rOVA is added to additional ingredients where at least one ingredient is a dairy ingredient or dairy-derived ingredient (such as milk, cream, whey, and butter). In some embodiments, rOVA is added to additional ingredients to create a beverage that does not contain any native egg protein, native egg white or native egg. In some embodiments, rOVA is an ingredient in a beverage that does not contain any animal-derived ingredients, such as one that does not contain any native egg-derived or any dairy-derived ingredients. Examples of such non-dairy derived drinks include nut milks, such as soy milk or almond milk. rOVA can also be used to create beverage additions, such as creamer or “milk” to provide protein, flavor, texture and mouthfeel to a beverage such as a coffee, tea, alcohol-based beverages or cocoa. In some embodiments, rOVA is present in a beverage ingredient or beverage addition in an amount between about 0.1% and 20% w/w or w/v.

In some embodiments herein, rOVA can be used to prepare a dairy-like product such as yogurt, cheese or butter. Dairy products with rOVA can include other animal-based dairy components or proteins. In some embodiments, dairy products prepared with rOVA do not include any animal-based ingredients.

Preparations of dessert products can be prepared using rOVA. In dessert products rOVA can provide one or more characteristics such as creamy texture, low fat content, odor neutrality, flavor, mouthfeel, texture, binding, and nutritional value. rOVA may be present in an ingredient or set of ingredients that is used to prepare a dessert product. Exemplary dessert products suitable for preparation with rOVA include a mousse, a cheesecake, a custard, a pudding, a popsicle and an ice cream. In some embodiments, dessert products prepared to include rOVA are vegan, vegetarian or dairy-free. Dessert products that include rOVA can have an amount of rOVA that is between about 0.1% and about 10% rOVA w/w or w/v.

rOVA can be used to prepare a snack food, such as a protein bar, an energy bar, a nutrition bar or a granola bar. The rOVA can provide characteristics to the snack food including one or more of binding, protein supplementation, flavor neutrality, odor neutrality, coating and mouth feel. In some embodiments, rOVA is added to a preparation of a snack food in an amount between about 0.1% and 30% w/w or w/v.

rOVA can be used for nutritional supplements such as in parenteral nutrition, protein drink supplements, protein shakes where rOVA provides a high protein supplement. In some embodiments, rOVA can be added to such compositions in an amount between about 10% and 30% w/w or w/v.

In some embodiments, rOVA compositions can be used as an egg-replacer and an egg white-replacer. rOVA can be mixed or combined with at least one additional component to form the egg white replacer. rOVA can provide one or more characteristics to the egg-replacer or egg white-replacer, such as gelling, foaming, whipping, fluffing, binding, springiness, aeration, creaminess and cohesiveness. In some embodiments, characteristic is the same or better than a native egg or native egg white provided in the same amount or concentration (w/w or w/v). In some embodiments, the egg-replacer or egg white-replacer, does not contain any egg, egg white, protein extracted or isolated from egg.

The rOVA-containing food ingredient and food products, such as described herein, can contain additional ingredients or components. For example, rOVA compositions can be prepared with an additional component such as one or more of a sweetener, a gum, a flavoring, a thickener, an acidulant and an emulsifier. Other ingredients such as flour, grains, oils and fats, fiber, fruit and vegetables can be combined with rOVA. Such rOVA compositions can be vegan, vegetarian, halal, kosher and animal-free, or a combination thereof. In some embodiments, rOVA can be a food ingredient or prepared for a food product that is normally animal based or normally contains animal-derived components, such as meat, dairy or eggs.

Compositions including rOVA including food ingredients and food products can be compatible with one or more steps of consumables preparation such as heated, baked, grilled, roasted, braised, microwaved, broiled, boiled, steamed, extruded, deep fried, or pan-fried, or processed using ohmic heating, Sue Vide, freezing, chilling, blanching, packaging, canning, bleaching, enriching, drying, pressing, grinding, mixing, par cooking, cooking, proofing, marinating, cutting, slicing, dicing, crushing, shredding, chopping, shaking, coring, spiralizing, rolling, juicing, straining, filtering, kneading, whisking, beating, whipping, grating, stuffing, peeling, smoking, curing, salting, preserving, pickling, fermenting, homogenizing, pasteurizing, sterilizing, irradiating, cold plasma processing, high pressure processing, pulse electric field processing, microwave assisted thermal sterilization, stabilizing, blending, pureeing, fortifying, refining, hydrogenating, aging, extending shelf life, or adding enzymes.

Food ingredients and food products prepared with rOVA can be essentially free of any microbial cells or microbial cell debris. For instance, rOVA may be secreted from a microbial host cell and isolated from microbial cells, culture media and/or microbial cell debris.

In some embodiments, rOVA may be prepared as a whole cell extract or fractionated extract such that an rOVA composition contains microbial cells and/or microbial cell components.

In one embodiment, an rOVA composition is prepared for animal consumption where the rOVA is present in a whole cell extract or fractionated extract such that an rOVA composition contains microbial cells and/or microbial cell components. In some embodiments, an rOVA composition is prepared for animal consumption where rOVA is isolated from microbial cells, culture media and microbial cell debris. Exemplary compositions for animal consumption can include a pet food, an animal feed, a chewy treat, bone broth, smoothie or other liquid for animal nutrition and a solid nutritional supplement suitable for animal consumption. In these cases, the microbial cell extract or microbial cell debris may provide additional nutritional value.

Animals which may consume rOVA compositions can include companion animals (e.g., dog, cat, horse), farm animals, exotic animals (lion, tiger, zebra) as well as livestock (such as cow, pig, sheep, goat). rOVA compositions as described herein can also be used for aquaculture (such as for fish and shellfish) and for avian nutrition (such as for bird pets, zoo birds, wild birds, fowl and birds raised for human and animal food).

In some embodiments of the consumable food compositions described herein, the composition is essentially free of animal-derived components, whey protein, caseinate, fat, lactose, hydrolyzed lactose, soy protein, collagen, hydrolyzed collagen, or gelatin, or any combination thereof. A composition described herein may be essentially free of cholesterol, glucose, fat, saturated fat, trans fat, or any combination thereof. In some cases, a composition described herein comprises less than 10%, 5%, 4%, 3%, 2%, 1%, or 0.5% fat by dry weight. In some embodiments, the composition may be fat-containing (e.g., such as a mayonnaise and commercial mayonnaise substitutes) and such composition may include up to about 60% fat or a reduced-fat composition (e.g., reduced fat mayonnaise and commercial mayonnaise substitutes) and such composition may include lesser percentages of fat. A composition that free of an animal-derived component can be considered vegetarian and/or vegan.

In some embodiments, an rOVA powder composition comprises less than 5% ash. The term “ash” is an art-known term and represents inorganics such as one or more ions, elements, minerals, and/or compounds. In some cases, the rOVA powder composition comprises less than 5%, 4.5%, 4%, 3.5%, 3%, 2.5%, 2%, 1.5%, 1%, 0.75%, 0.5%, 0.25% or 0.1% ash weight per total weight (w/w) and/or weight per total volume (w/v).

In some embodiments, the moisture content of an rOVA powder composition may be less than 15%. The rOVA powder composition may have less than 15%, 12%, 10%, 8%, 6%, 5%, 3%, 2% or 1% moisture weight per total weight (w/w) and/or weight per total volume (w/v). In some embodiments, the carbohydrate content of an rOVA powder composition may be less than 30%. The rOVA powder composition may have less than 30%, 27%, 25%, 22%, 20%, 17%, 15%, 12%, 10%, 8%, 5%, 3% or 1% carbohydrate content w/w or w/v.

### Sensory Neutrality and Improved Sensory Appeal

In some embodiments, in addition to the egg-white like properties, the addition of rOVA to a consumable food composition provides increased protein nutritional content, sensory neutrality or an improved sensory appeal as compared to other proteins in such compositions. As used herein “sensory neutrality” refers to the absence of a strong or distinctive taste, odor (smell) or combination of taste and smell, as well as texture, mouth-feel, aftertaste and color. A sensory panel such as one described in Kemp et al. 2009 may be used by a trained sensory analyst. Sensory neutrality may provide an improved sensory appeal to a taster, such as a tester of foods or a consumer, when a consumable food composition containing rOVA is compared with another like composition that has a different protein such as nOVA, whey protein, pea protein, soy protein, whole egg or egg white protein at the same concentration.

In some embodiments, rOVA when added to a consumable food composition is substantially odorless, such as measured by a trained sensory analyst, in comparison with different solutions/products with a different protein component present in an equal concentration to the rOVA containing solution/product, for example, in the comparison is whey, soy, collagen, pea, egg white solid isolates and/or nOVA. In some embodiments of the rOVA compositions described herein, such compositions are essentially odorless at a protein concentration between about 0.5-1%, 1%-5%, 5-10%, 10-15%, 15-20%, 20-25%, 25-30% rOVA weight per total weight (w/w) and/or weight per total volume (w/v) or at a protein concentration of about 0.1, 1, 5, 10, 11, 12, 13, 14, 15, 16, 17, 18, 19, 20, 21, 22, 23, 24, 25, 26, 27, 28, 29 or 30 g of total rOVA protein per 100 mL solution (e.g., per 100 mL water).

In some embodiments, the addition of rOVA to a consumable food composition also provides a neutral taste in addition to the characteristics such as egg-white like properties and increased protein nutrition content. A neutral taste can be measured for example, by a trained sensory analyst in comparison with solutions containing a different protein present in an equal concentration to the rOVA, for example, whey, soy, collagen, pea, whole egg, and egg white solid isolates (including native OVA).

In some embodiments, the addition of rOVA provides a reduction in a certain odor and/or taste that is associated with other proteins or egg-whites. For example, addition of rOVA has less of an “egg-like” odor or taste as compared to the addition of whole egg, fractionated egg or egg-white to a consumable food composition. In some embodiments, addition of rOVA has less of a metallic odor or taste as compared to other protein sources.

In some embodiments, the addition of rOVA has an improved mouth-feel as compared to the addition of other protein sources used to produce egg-white like properties. For example, the addition of rOVA is less grainy or has less precipitates or solids as compared to other protein sources.

In some embodiments, the addition of rOVA has an improved texture, for example, as compared to other available supplemental protein sources.

A consumable composition with rOVA may also have an improved sensory appeal as compared to the composition without rOVA or with a different protein present in an equal concentration to the rOVA. Such improved sensory appeal may relate to taste and/or smell. Taste and smell can be measured, for example, by a trained sensory analyst. In some instances, a sensory analyst compares a consumable composition with rOVA to one without it or with a different protein or protein source in an equivalent amount.

As described herein, a consumable composition herein can be in a liquid form. A liquid form can be an intermediate product such as soluble rOVA solution. In some cases, a liquid form can be a final product, such as a beverage comprising rOVA. Example of different types of beverages contemplated herein include: a juice, a soda, a soft drink, a flavored water, a protein water, a fortified water, a carbonated water, a nutritional drink, an energy drink, a sports drink, a recovery drink, an alcohol-based drink, a heated drink, a coffee-based drink, a tea-based drink, a plant-based milk, a nut milk, a milk based drink, a non-dairy, plant based mild drink, infant formula drink, and a meal replacement drink.

### pH of Compositions

The pH of an rOVA composition may be 3.5 to 8. The pH of an rOVA composition may be at least 3.5. The pH of an rOVA composition may be at most 8. The pH of an rOVA composition may be 3.5 to 4, 3.5 to 4.5, 3.5 to 5, 3.5 to 5.5, 3.5 to 6, 3.5 to 6.5, 3.5 to 7, 3.5 to 7.5, 3.5 to 8, 4 to 4.5, 4 to 5, 4 to 5.5, 4 to 6, 4 to 6.5, 4 to 7, 4 to 7.5, 4 to 8, 4.5 to 5, 4.5 to 5.5, 4.5 to 6, 4.5 to 6.5, 4.5 to 7, 4.5 to 7.5, 4.5 to 8, 5 to 5.5, 5 to 6, 5 to 6.5, 5 to 7, 5 to 7.5, 5 to 8, 5.5 to 6, 5.5 to 6.5, 5.5 to 7, 5.5 to 7.5, 5.5 to 8, 6 to 6.5, 6 to 7, 6 to 7.5, 6 to 8, 6.5 to 7, 6.5 to 7.5, 6.5 to 8, 7 to 7.5, 7 to 8, or 7.5 to 8. The pH of an rOVA composition may be 3.5, 4, 4.5, 5, 5.5, 6, 6.5, 7, 7.5, or 8. An rOVA composition with a pH between 3.5 to 7 may have one or more improved functionalities as compared to nOVA, egg white or egg-white substitute compositions.

The pH of an rOVA composition may be 2 to 3.5. The pH of an rOVA composition may be at least 2. The pH of an rOVA composition may be at most 3.5. The pH of an rOVA composition may be 2 to 2.5, 2 to 3, 2 to 3.5, 2.5 to 3, 2.5 to 3.5, or 3 to 3.5. The pH of an rOVA composition may be 2, 2.5, 3, or 3.5.

The pH of an rOVA composition may be 7 to 12. The pH of an rOVA composition may be at least 7. The pH of an rOVA composition may be at most 12. The pH of an rOVA composition may be 7 to 7.5, 7 to 8, 7 to 8.5, 7 to 9, 7 to 9.5, 7 to 10, 7 to 10.5, 7 to 11, 7 to 11.5, 7 to 12, 7.5 to 8, 7.5 to 8.5, 7.5 to 9, 7.5 to 9.5, 7.5 to 10, 7.5 to 10.5, 7.5 to 11, 7.5 to 11.5, 7.5 to 12, 8 to 8.5, 8 to 9, 8 to 9.5, 8 to 10, 8 to 10.5, 8 to 11, 8 to 11.5, 8 to 12, 8.5 to 9, 8.5 to 9.5, 8.5 to 10, 8.5 to 10.5, 8.5 to 11, 8.5 to 11.5, 8.5 to 12, 9 to 9.5, 9 to 10, 9 to 10.5, 9 to 11, 9 to 11.5, 9 to 12, 9.5 to 10, 9.5 to 10.5, 9.5 to 11, 9.5 to 11.5, 9.5 to 12, 10 to 10.5, 10 to 11, 10 to 11.5, 10 to 12, 10.5 to 11, 10.5 to 11.5, 10.5 to 12, 11 to 11.5, 11 to 12, or 11.5 to 12. The pH of an rOVA composition may be 7, 7.5, 8, 8.5, 9, 9.5, 10, 10.5, 11, 11.5, or 12.

In some embodiments, the pH of rOVA may be adjusted prior to its inclusion in a composition or its use as an ingredient. In some embodiments, the pH of rOVA is adjusted during the purification and/or isolation processes. In some embodiments, the pH of the rOVA for use in an ingredient or in production of a food product composition is adjusted to between about 3.5 to about 7.0. In some cases, the pH of rOVA may be adjusted to more than one pH during the production process. For example, rOVA may be expressed in a host cell such as a a microbial cell, and in some cases the rOVA is secreted by the host cell into the growth media (e.g., liquid media). rOVA is separated from the host cells and such separation step may be performed at a selected pH, for example at a pH of about 3.5. In some cases, the rOVA at such separation pH may not be soluble or may not be fully soluble and the pH is adjusted to a higher pH, such as about pH 12. The rOVA may then be adjusted to a final pH between about 3.5 and about 7.0. Separation of rOVA from other components of the host cells or other components of the liquid media can include one or more of ion exchange chromatography, such as cation exchange chromatography and/or anion exchange chromatography, filtration and ammonium sulfate precipitation.

### Additional Components of Compositions

The consumable food compositions containing rOVA disclosed herein and the methods of making such compositions may including adding or mixing the rOVA with one or more ingredients. For example, food additives may be added in or mixed with the compositions. Food additives can add volume and/or mass to a composition. A food additive may improve functional performance and/or physical characteristics. For example, a food additive may prevent gelation or increased viscosity due to the lipid portion of the lipoproteins in the freeze-thaw cycle. An anticaking agent may be added to make a free-flowing composition. Carbohydrates can be added to increase resistance to heat damage, e.g., less protein denaturation during drying and improve stability and flowability of dried compositions. Food additives include, but are not limited to, food coloring, pH adjuster, natural flavoring, artificial flavoring, flavor enhancer, batch marker, food acid, filler, anticaking agent (e.g., sodium silico aluminate), antigreening agent (e.g., citric acid), food stabilizer, foam stabilizer or binding agent, antioxidant, acidity regulatory, bulking agent, color retention agent, whipping agent (e.g., ester-type whipping agent, triethyl citrate, sodium lauryl sulfate), emulsifier (e.g., lecithin), humectant, thickener, excipient, solid diluent, salts, nutrient, sweetener, glazing agent, preservative, vitamin, dietary elements, carbohydrates, polyol, gums, starches, flour, oil, or bran.

Food coloring includes, but is not limited to, FD&C Yellow #5, FD&C Yellow #6, FD&C Red #40, FD&C Red #3, FD&C Blue No. 1, FD&C Blue No. 2, FD&C Green No. 3, carotenoids (e.g., saffron, β-carotene), anthocyanins, annatto, betanin, butterfly pea, caramel coloring, chlorophyllin, elderberry juice, lycopene, carmine, pandan, paprika, turmeric, curcuminoids, quinoline yellow, carmoisine, Ponceau 4R, Patent Blue V, and Green S.

Ingredients for pH adjustment include, but are not limited to, Tris buffer, potassium phosphate, sodium hydroxide, potassium hydroxide, citric acid, sodium citrate, sodium bicarbonate, and hydrochloric acid.

Salts include, but are not limited, to acid salts, alkali salts, organic salts, inorganic salts, phosphates, chloride salts, sodium salts, sodium chloride, potassium salts, potassium chloride, magnesium salts, magnesium chloride, magnesium perchlorate, calcium salts, calcium chloride, ammonium chloride, iron salts, iron chlorides, zinc salts, and zinc chloride.

Nutrient includes, but is not limited to, macronutrient, micronutrient, essential nutrient, non-essential nutrient, dietary fiber, amino acid, essential fatty acids, omega-3 fatty acids, and conjugated linoleic acid.

Sweeteners include, but are not limited to, sugar substitute, artificial sweetener, acesulfame potassium, advantame, alitame, aspartame, sodium cyclamate, dulcin, glucin, neohesperidin dihydrochalcone, neotame, P-4000, saccharin, aspartame-acesulfame salt, sucralose, brazzein, curculin, glycyrrhizin, glycerol, inulin, mogroside, mabinlin, malto-oligosaccharide, mannitol, miraculin, monatin, monellin, osladin, pentadin, stevia, trilobatin, and thaumatin.

Carbohydrates include, but are not limited to, sugar, sucrose, glucose, fructose, galactose, lactose, maltose, mannose, allulose, tagatose, xylose, arabinose, high fructose corn syrup, high maltose corn syrup, corn syrup (e.g., glucose-free corn syrup), sialic acid, monosaccharides, disaccharides, polysaccharides (e.g., polydextrose, maltodextrin), and starch.

Polyols include, but are not limited to, xylitol, maltitol, erythritol, sorbitol, threitol, arabitol, hydrogenated starch hydrolysates, isomalt, lactitol, mannitol, and galactitol (dulcitol).

Gums include, but are not limited to, gum arabic, gellan gum, guar gum, locust bean gum, acacia gum, cellulose gum, and xanthan gum.

Vitamins include, but are not limited to, niacin, riboflavin, pantothenic acid, thiamine, folic acid, vitamin A, vitamin B6, vitamin B12, vitamin D, vitamin E, lutein, zeaxanthin, choline, inositol, and biotin.

Dietary elements include, but are not limited to, calcium, iron, magnesium, phosphorus, potassium, sodium, zinc, copper, manganese, selenium, chlorine, iodine, sulfur, cobalt, molybdenum, nickel, and bromine.

### Definitions

The terminology used herein is for the purpose of describing particular cases only and is not intended to be limiting.

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

As used in the specification and claims, the singular forms “a”, “an” and “the” include plural references unless the context clearly dictates otherwise.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “about” a number refers to that number plus or minus 10% of that number and/or within one standard deviation (plus or minus) from that number. The term “about” a range refers to that range minus 10% of its lowest value and plus 10% of its greatest value and that range minus one standard deviation its lowest value and plus one standard deviation of its greatest value.

Ranges can be expressed herein as from “about” or “approximately” one particular value, and/or to “about” or “approximately” another particular value. When such a range is expressed, another case includes from the one particular value and/or to the other particular value. Similarly, when values are expressed as approximations, by use of the antecedent “about” or “approximately”, it will be understood that the particular value forms another case. It will be further understood that the endpoints of each of the ranges are significant both in relation to the other endpoint, and independently of the other endpoint. The term “about” or “approximately” as used herein refers to a range that is 15% plus or minus from a stated numerical value within the context of the particular usage. For example, about 10 would include a range from 8.5 to 11.5. The term “about” or “approximately” also accounts for typical error or imprecision in measurement of values.

Moreover, it should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

The terms “including”, “includes”, “having”, “has”, “with”, or variants thereof are used in either the detailed description and/or the claims, such terms are intended to be inclusive in a manner similar to the term “comprising”.

Any aspect or embodiment described herein can be combined with any other aspect or embodiment as disclosed herein.

### REFERENCES

Upadhyay V., Singh A., Panda A. (2016) Purification of recombinant ovalbumin from inclusion bodies of Escherichia coli, Protein Expression and Purification, 117, 52-58.
Demain A. L., Vaishnav P. (2009). Production of recombinant proteins by microbes and higher organisms. Biotechnol. Adv. 27 297-306
Jarvio, N., Parviainen, T., Maljanen, N L. et al. Ovalbumin production using Trichoderma reesei culture and low-carbon energy could mitigate the environmental impacts of chicken-egg-derived ovalbumin. Nat Food 2, 1005-1013 (2021).
US2022/0039443, US2021/0337826, and US2018/0355020.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

Illustrative OVA amino acid sequences contemplated herein are provided in the below Table 1 as SEQ ID NO: 1 to SEQ ID NO: 74.

TABLE 1

| SEQ | | |
|---|---|---|
| ID | | |
| NO | Name | Sequence |
| 1 | Chicken | MRFPSIFTAVLFAASSALAAPVNTTTEDETAQIPAEAVIGYSDLEGDFDVAVLPFS |
| | Ovalbumin with | NSTNNGLLFINTTIASIAAKEEGVSLDKREAEAGSIGAASMEFCFDVFKELKVHHA |
| | bolded signal | NENIFYCPIAIMSALAMVYLGAKDSTRTQINKVVRFDKLPGFGDSIEAQCGTSVNV |
| | sequence | HSSLRDILNQITKPNDVYSFSLASRLYAEERYPILPEYLQCVKELYRGGLEPINFQ |
| | | TAADQARELINSWVESQTNGIIRNVLQPSSVDSQTAMVLVNAIVFKGLWEKAFKDE |
| | | DTQAMPFRVTEQESKPVQMMYQIGLFRVASMASEKMKILELPFASGTMSMLVLLPD |
| | | EVSGLEQLESIINFEKLTEWTSSNVMEERKIKVYLPRMKMEEKYNLTSVLMAMGIT |
| | | DVFSSSANLSGISSAESLKISQAVHAAHAEINEAGREVVGSAEAGVDAASVSEEFR |
| | | ADHPFLFCIKHIATNAVLFFGRCVSP |
| 2 | Chicken OVA | EAEAGSIGAASMEFCFDVFKELKVHHANENIFYCPIAIMSALAMVYLGAKDSTRTQ |
| | sequence as | INKVVRFDKLPGFGDSIEAQCGTSVNVHSSLRDILNQITKPNDVYSFSLASRLYAE |
| | secreted from | ERYPILPEYLQCVKELYRGGLEPINFQTAADQARELINSWVESQINGIIRNVLQPS |
| | pichia | SVDSQTAMVLVNAIVFKGLWEKAFKDEDTQAMPFRVTEQESKPVQMMYQIGLFRVA |
| | | SMASEKMKILELPFASGTMSMLVLLPDEVSGLEQLESIINFEKLTEWTSSNVMEER |
| | | KIKVYLPRMKMEEKYNLTSVLMAMGITDVFSSSANLSGISSAESLKISQAVHAAHA |
| | | EINEAGREVVGSAEAGVDAASVSEEFRADHPFLFCIKHIATNAVLFFGRCVSP |
| 3 | Predicted | MRVPAQLLGLLLLWLPGARCGSIGAASMEFCFDVFKELKVHHANENIFYCPIAIMS |
| | Ovalbumin | ALAMVYLGAKDSTRTQINKVVRFDKLPGFGDSIEAQCGTSVNVHSSLRDILNQITK |
| | [Achromobacter | PNDVYSFSLASRLYAEERYPILPEYLQCVKELYRGGLEPINFQTAADQARELINSW |
| | denitrificans] | VESQTNGIIRNVLQPSSVDSQTAMVLVNAIVFKGLWEKAFKDEDTQAMPFRVTEQE |
| | | SKPVQMMYQIGLFRVASMASEKMKILELPFASGTMSMLVLLPDEVSGLEQLESIIN |
| | | FEKLTEWTSSNVMEERKIKVYLPRMKMEEKYNLTSVLMAMGITDVFSSSANLSGIS |
| | | SAESLKISQAVHAAHAEINEAGREVVGSAEAGVDAASVSEEFRADHPFLFCIKHIA |
| | | TNAVLFFGRCVSPLEIKRAAAHHHHHH |
| 4 | OLLAS epitope- | MTSGFANELGPRLMGKLTMGSIGAASMEFCFDVFKELKVHHANENIFYCPIAIMSA |
| | tagged | LAMVYLGAKDSTRTQINKVVRFDKLPGFGDSIEAQCGTSVNVHSSLRDILNQITKP |
| | ovalbumin | NDVYSFSLASRLYAEERYPILPEYLQCVKELYRGGLEPINFQTAADQARELINSWV |
| | | ESQTNGIIRNVLQPSSVDSQTAMVLVNAIVFKGLWEKTFKDEDTQAMPFRVTEQES |
| | | KPVQMMYQIGLFRVASMASEKMKILELPFASGTMSMLVLLPDEVSGLEQLESIINF |
| | | EKLTEWTSSNVMEERKIKVYLPRMKMEEKYNLTSVLMAMGITDVESSSANLSGISS |
| | | AESLKISQAVHAAHAEINEAGREVVGSAEAGVDAASVSEEFRADHPFLFCIKHIAT |
| | | NAVLFFGRCVSPSR |
| 5 | Serpin family | MGGRRVRWEVYISRAGYVNRQIAWRRHHRSLTMRVPAQLLGLLLLWLPGARCGSIG |
| | protein | AASMEFCFDVFKELKVHHANENIFYCPIAIMSALAMVYLGAKDSTRTQINKVVRFD |
| | [Achromobacter | KLPGFGDSIEAQCGTSVNVHSSLRDILNQITKPNDVYSFSLASRLYAEERYPILPE |
| | denitrificans] | YLQCVKELYRGGLEPINFQTAADQARELINSWVESQTNGIIRNVLQPSSVDSQTAM |
| | | VLVNAIVFKGLWEKAFKDEDTQAMPFRVTEQESKPVQMMYQIGLFRVASMASEKMK |
| | | ILELPFASGTMSMLVLLPDEVSGLEQLESIINFEKLTEWTSSNVMEERKIKVYLPR |
| | | MKMEEKYNLTSVLMAMGITDVFSSSANLSGISSAESLKISQAVHAAHAEINEAGRE |
| | | VVGSAEAGVDAASVSEEFRADHPFLFCIKHIATNAVLFFGRCVSPLEIKRAAAHHH |
| | | HHH |
| 6 | PREDICTED: | MGSIGAVSMEFCFDVFKELKVHHANENIFYSPFTIISALAMVYLGAKDSTRTQINK |
| | ovalbumin | VVRFDKLPGFGDSVEAQCGTSVNVHSSLRDILNQITKPNDVYSFSLASRLYAEETY |
| | isoform X1 | PILPEYLQCVKELYRGGLESINFQTAADQARGLINSWVESQTNGMIKNVLQPSSVD |
| | [Meleagris | SQTAMVLVNAIVFKGLWEKAFKDEDTQAIPFRVTEQESKPVQMMYQIGLFKVASMA |
| | gallopavo] | SEKMKILELPFASGTMSMWVLLPDEVSGLEQLETTISFEKMTEWISSNIMEERRIK |
| | | VYLPRMKMEEKYNLTSVLMAMGITDLFSSSANLSGISSAGSLKISQAVHAAYAEIY |
| | | EAGREVIGSAEAGADATSVSEEFRVDHPFLYCIKHNLTNSILFFGRCISP |
| 7 | Ovalbumin | MGSIGAVSMEFCFDVFKELKVHHANENIFYSPFTIISALAMVYLGAKDSTRTQINK |
| | precursor | VVRFDKLPGFGDSVEAQCGTSVNVHSSLRDILNQITKPNDVYSFSLASRLYAEETY |
| | [Meleagris | PILPEYLQCVKELYRGGLESINFQTAADQARGLINSWVESQTNGMIKNVLQPSSVD |
| | gallopavo] | SQTAMVLVNAIVFKGLWEKAFKDEDTQAIPFRVTEQESKPVQMMYQIGLFKVASMA |
| | | SEKMKILELPFASGTMSMWVLLPDEVSGLEQLETTISFEKMTEWISSNIMEERRIK |
| | | VYLPRMKMEEKYNLTSVLMAMGITDLFSSSANLSGISSAGSLKISQAAHAAYAEIY |
| | | EAGREVIGSAEAGADATSVSEEFRVDHPFLYCIKHNLTNSILFFGRCISP |
| 8 | Hypothetical | YYRVPCMVLCTAFHPYIFIVLLFALDNSEFTMGSIGAVSMEFCFDVFKELRVHHPN |
| | protein | ENIFFCPFAIMSAMAMVYLGAKDSTRTQINKVIRFDKLPGFGDSTEAQCGKSANVH |
| | [Bambusicola | SSLKDILNQITKPNDVYSFSLASRLYADETYSIQSEYLQCVNELYRGGLESINFQT |
| | thoracicus] | AADQARELINSWVESQTNGIIRNVLQPSSVDSQTAMVLVNAIVFRGLWEKAFKDED |
| | | TQTMPFRVTEQESKPVQMMYQIGSFKVASMASEKMKILELPLASGTMSMLVLLPDE |
| | | VSGLEQLETTISFEKLTEWTSSNVMEERKIKVYLPRMKMEEKYNLTSVLMAMGITD |
| | | LFRSSANLSGISLAGNLKISQAVHAAHAEINEAGRKAVSSAEAGVDATSVSEEFRA |
| | | DRPFLFCIKHIATKVVFFFGRYTSP |
| 9 | Egg albumin | MGSIGAASMEFCFDVFKELKVHHANDNMLYSPFAILSTLAMVFLGAKDSTRTQINK |
| | | VVHFDKLPGFGDSIEAQCGTSVNVHSSLRDILNQITKQNDAYSFSLASRLYAQETY |
| | | TVVPEYLQCVKELYRGGLESVNFQTAADQARGLINAWVESQTNGIIRNILQPSSVD |
| | | SQTAMVLVNAIAFKGLWEKAFKAEDTQTIPFRVTEQESKPVQMMYQIGSFKVASMA |
| | | SEKMKILELPFASGTMSMLVLLPDDVSGLEQLESIISFEKLTEWTSSSIMEERKVK |
| | | VYLPRMKMEEKYNLTSLLMAMGITDLFSSSANLSGISSVGSLKISQAVHAAHAEIN |
| | | EAGRDVVGSAEAGVDATEEFRADHPFLFCVKHIETNAILLFGRCVSP |
| 10 | Ovalbumin | MASIGAVSTEFCVDVYKELRVHHANENIFYSPFTIISTLAMVYLGAKDSTRTQINK |
| | isoform X2 | VVRFDKLPGFGDSIEAQCGTSVNVHSSLRDILNQITKPNDVYSFSLASRLYAEETY |
| | [Numida | PILPEYLQCVKELYRGGLESINFQTAADQARELINSWVESQTSGIIKNVLQPSSVN |
| | meleagris] | SQTAMVLVNAIYFKGLWERAFKDEDTQAIPFRVTEQESKPVQMMSQIGSFKVASVA |
| | | SEKVKILELPFVSGTMSMLVLLPDEVSGLEQLESTISTEKLTEWTSSSIMEERKIK |
| | | VFLPRMRMEEKYNLTSVLMAMGMTDLFSSSANLSGISSAESLKISQAVHAAYAEIY |
| | | EAGREVVSSAEAGVDATSVSEEFRVDHPFLLCIKHNPTNSILFFGRCISP |
| 11 | Ovalbumin | MALCKAFHPYIFIVLLFDVDNSAFTMASIGAVSTEFCVDVYKELRVHHANENIFYS |
| | isoform X1 | PFTIISTLAMVYLGAKDSTRTQINKVVRFDKLPGFGDSIEAQCGTSVNVHSSLRDI |
| | [Numida | LNQITKPNDVYSFSLASRLYAEETYPILPEYLQCVKELYRGGLESINFQTAADQAR |
| | meleagris] | ELINSWVESQTSGIIKNVLQPSSVNSQTAMVLVNAIYFKGLWERAFKDEDTQAIPF |
| | | RVTEQESKPVQMMSQIGSFKVASVASEKVKILELPFVSGTMSMLVLLPDEVSGLEQ |
| | | LESTISTEKLTEWTSSSIMEERKIKVFLPRMRMEEKYNLTSVLMAMGMTDLFSSSA |
| | | NLSGISSAESLKISQAVHAAYAEIYEAGREVVSSAEAGVDATSVSEEFRVDHPFLL |
| | | CIKHNPTNSILFFGRCISP |
| 12 | PREDICTED: | MGSIGAASMEFCFDVFKELKVHHANDNMLYSPFAILSTLAMVFLGAKDSTRTQINK |
| | Ovalbumin | VVHFDKLPGFGDSIEAQCGTSANVHSSLRDILNQITKQNDAYSFSLASRLYAQETY |
| | isoform X2 | TVVPEYLQCVKELYRGGLESVNFQTAADQARGLINAWVESQTNGIIRNILQPSSVD |
| | [Coturnix | SQTAMVLVNAIAFKGLWEKAFKAEDTQTIPFRVTEQESKPVQMMHQIGSFKVASMA |
| | japonica] | SEKMKILELPFASGTMSMLVLLPDDVSGLEQLESTISFEKLTEWTSSSIMEERKVK |
| | | VYLPRMKMEEKYNLTSLLMAMGITDLFSSSANLSGISSVGSLKISQAVHAAYAEIN |
| | | EAGRDVVGSAEAGVDATEEFRADHPFLFCVKHIETNAILLFGRCVSP |
| 13 | PREDICTED: | MGLCTAFHPYIFIVLLFALDNSEFTMGSIGAASMEFCFDVFKELKVHHANDNMLYS |
| | ovalbumin | PFAILSTLAMVFLGAKDSTRTQINKVVHFDKLPGFGDSIEAQCGTSANVHSSLRDI |
| | isoform X1 | LNQITKONDAYSFSLASRLYAQETYTVVPEYLQCVKELYRGGLESVNFQTAADQAR |
| | [Coturnix | GLINAWVESQTNGIIRNILQPSSVDSQTAMVLVNAIAFKGLWEKAFKAEDTQTIPF |
| | japonica] | RVTEQESKPVQMMHQIGSFKVASMASEKMKILELPFASGTMSMLVLLPDDVSGLEQ |
| | | LESTISFEKLTEWTSSSIMEERKVKVYLPRMKMEEKYNLTSLLMAMGITDLFSSSA |
| | | NLSGISSVGSLKISQAVHAAYAEINEAGRDVVGSAEAGVDATEEFRADHPFLFCVK |
| | | HIETNAILLFGRCVSP |
| 14 | Egg albumin | MGSIGAASMEFCFDVFKELKVHHANDNMLYSPFAILSTLAMVFLGAKDSTRTQINK |
| | | VVHFDKLPGFGDSIEAQCGTSANVHSSLRDILNQITKQNDAYSFSLASRLYAQETY |
| | | TVVPEYLQCVKELYRGGLESVNFQTAADQARGLINAWVESQTNGIIRNILQPSSVD |
| | | SQTAMVLVNAIAFKGLWEKAFKAEDTQTIPFRVTEQESKPVQMMHQIGSFKVASMA |
| | | SEKMKILELPFASGTMSMLVLLPDDVSGLEQLESTISFEKLTEWTSSSIMEERKVK |
| | | VYLPRMKMEEKYNLTSLLMAMGITDLFSSSANLSGISSVGSLKIPQAVHAAYAEIN |
| | | EAGRDVVGSAEAGVDATEEFRADHPFLFCVKHIETNAILLFGRCVSP |
| 15 | ovalbumin [Anas | MGSIGAASTEFCFDVFRELRVQHVNENIFYSPFSIISALAMVYLGARDNTRTQIDK |
| | platyrhynchos] | VVHFDKLPGFGESMEAQCGTSVSVHSSLRDILTQITKPSDNFSLSFASRLYAEETY |
| | | AILPEYLQCVKELYKGGLESISFQTAADQARELINSWVESQTNGIIKNILQPSSVD |
| | | SQTTMVLVNAIYFKGMWEKAFKDEDTQAMPFRMTEQESKPVQMMYQVGSFKVAMVT |
| | | SEKMKILELPFASGMMSMFVLLPDEVSGLEQLESTISFEKLTEWTSSTMMEERRMK |
| | | VYLPRMKMEEKYNLTSVFMALGMTDLFSSSANMSGISSTVSLKMSEAVHAACVEIF |
| | | EAGRDVVGSAEAGMDVTSVSEEFRADHPFLFFIKHNPTNSILFFGRWMSP |
| 16 | PREDICTED: | MGSIGAASTEFCFDVFRELKVQHVNENIFYSPLSIISALAMVYLGARDNTRTQIDQ |
| | ovalbumin-like | VVHFDKIPGFGESMEAQCGTSVSVHSSLRDILTEITKPSDNFSLSFASRLYAEETY |
| | [Anser cygnoides | TILPEYLQCVKELYKGGLESISFQTAADQARELINSWVESQTNGIIKNILQPSSVD |
| | domesticus] | SQTTMVLVNAIYFKGMWEKAFKDEDTQTMPFRMTEQESKPVQMMYQVGSFKLATVT |
| | | SEKVKILELPFASGMMSMCVLLPDEVSGLEQLETTISFEKLTEWTSSTMMEERRMK |
| | | VYLPRMKMEEKYNLTSVFMALGMTDLFSSSANMSGISSTVSLKMSEAVHAACVEIF |
| | | EAGRDVVGSAEAGMDVTSVSEEFRADHPFLFFIKHNPSNSILFFGRWISP |
| 17 | PREDICTED: | MGSIGAASTEFCFDVFKELKVQHVNENIFYSPLTIISALSMVYLGARENTRAQIDK |
| | Ovalbumin-like | VLHFDKMPGFGDTIESQCGTSVSIHTSLKDMFTQITKPSDNYSLSFASRLYAEETY |
| | [Aquila | PILPEYLQCVKELYKGGLETISFQTAAEQARELINSWVESQTNGMIKNILQPSSVD |
| | chrysaetos | PQTKMVLVNAIYFKGVWEKAFKDEDTQEVPFRVTEQESKPVQMMYQIGSFKVAVMA |
| | canadensis] | SEKMKILELPYASGQLSMLVLLPDDVSGLEQLESAITFEKLMAWTSSTTMEERKMK |
| | | VYLPRMKIEEKYNLTSVLMALGVTDLFSSSANLSGISSAESLKISKAVHEAFVEIY |
| | | EAGSEVVGSTEAGMEVTSVSEEFRADHPFLFLIKHNPTNSILFFGRCFSP |
| 18 | PREDICTED: | MGSIGAASTEFCFDVFKELKVQHVNENIFYSPLTIISALSMVYLGARENTRTQIDK |
| | Ovalbumin-like | VLHFDKMTGFGDTVESQCGTSVSIHTSLKDIFTQITKPSDNYSLSLASRLYAEETY |
| | [Haliaeetus | PILPEYLQCVKELYKGGLETVSFQTAAEQARELINSWVESQTNGMIKNILQPSSVD |
| | albicilla] | PQTKMVLVNAIYFKGVWEKAFKDEDTQEVPFRVTEQESKPVQMMYQIGSFKVAVMA |
| | | SEKMKILELPYASGQLSMLVLLPDDVSGLEQLESAITSEKLMEWTSSTTMEERKMK |
| | | VYLPRMKIEEKYNLTSVLMALGVTDLFSSSADLSGISSAESLKISKAVHEAFVEIY |
| | | EAGSEVVGSTEGGMEVTSVSEEFRADHPFLFLIKHKPTNSILFFGRCFSP |
| 19 | PREDICTED: | MGSIGAASTEFCFDVFKELKVQHVNENIFYSPLTIISALSMVYLGARENTRTQIDK |
| | Ovalbumin-like | VLHFDKMTGFGDTVESQCGTSVSIHTSLKDIFTQITKPSDNYSLSLASRLYAEETY |
| | [Haliaeetus | PILPEYLQCVKELYKGGLETVSFQTAAEQARELINSWVESQTNGMIKNILQPSSVD |
| | leucocephalus] | PQTKMVLVNAIYFKGVWEKAFKDEDTQEVPFRVTEQESKPVQMMYQIGSFKVAVMA |
| | | SEKMKILELPYASGQLSMLVLLPDDVSGLEQLESAITSEKLMEWTSSTTMEERKMK |
| | | VYLPRMKIEEKYNLTSVLMALGVTDLFSSSADLSGISSAESLKISKAVHEAFVEIY |
| | | EAGSEVVGSTEGGMEVTSFSEEFRADHPFLFLIKHKPTNSILFFGRCFSP |
| 20 | PREDICTED: | MGSIGAASTEFCFDVFKELKVQHVNENIFYSPLSIISALSMVYLGARENTRAQIDK |
| | Ovalbumin | VVHFDKITGFGETIESQCGTSVSVHTSLKDMFTQITKPSDNYSLSFASRLYAEETY |
| | [Fulmarus | PILPEYLQCVKELYKGGLETTSFQTAADQARELINSWVESQTNGMIKNILQPGSVD |
| | glacialis] | PQTEMVLVNAIYFKGMWEKAFKDEDTQAVPFRMTEQESKTVQMMYQIGSFKVAVMA |
| | | SEKMKILELPYASGELSMLVMLPDDVSGLEQLETAITFEKLMEWTSSNMMEERKMK |
| | | VYLPRMKMEEKYNLTSVLMALGVTDLFSSSANLSGISSAESLKMSEAVHEAFVEIY |
| | | EAGSEVVGSTGAGMEVTSVSEEFRADHPFLFLIKHNPTNSILFFGRCFSP |
| 21 | PREDICTED: | MGSIGAASTEFCFDVFKELRVQHVNENVCYSPLIIISALSLVYLGARENTRAQIDK |
| | Ovalbumin-like | VVHFDKITGFGESIESQCGTSVSVHTSLKDMFNQITKPSDNYSLSVASRLYAEERY |
| | [Chlamydotis | PILPEYLQCVKELYKGGLESISFQTAADQAREAINSWVESQTNGMIKNILQPSSVD |
| | macqueenii] | PQTEMVLVNAIYFKGMWQKAFKDEDTQAVPFRISEQESKPVQMMYQIGSFKVAVMA |
| | | AEKMKILELPYASGELSMLVLLPDEVSGLEQLENAITVEKLMEWTSSSPMEERIMK |
| | | VYLPRMKIEEKYNLTSVLMALGITDLFSSSANLSGISAEESLKMSEAVHQAFAEIS |
| | | EAGSEVVGSSEAGIDATSVSEEFRADHPFLFLIKHNATNSILFFGRCFSP |
| 22 | PREDICTED: | MGSISAASTEFCFDVFKELKVQHVNENIFYSPLSIISALSMVYLGARENTRAQIEK |
| | Ovalbumin like | VVHFDKITGFGESIESQCSTSVSVHTSLKDMFTQITKPSDNYSLSFASRFYAEETY |
| | [Nipponia | PILPEYLQCVKELYKGGLETINFRTAADQARELINSWVESQTNGMIKNILQPGSVD |
| | nippon] | PQTDMVLVNAIYFKGMWEKAFKDEDTQALPFRVTEQESKPVQMMYQIGSFKVAVLA |
| | | SEKVKILELPYASGQLSMLVLLPDDVSGLEQLETAITVEKLMEWTSSNNMEERKIK |
| | | VYLPRIKIEEKYNLTSVLMALGITDLFSSSANLSGISSAESLKVSEAIHEAFVEIY |
| | | EAGSEVAGSTEAGIEVTSVSEEFRADHPFLFLIKHNATNSILFFGRCFSP |
| 23 | PREDICTED: | MVSIGAASTEFCFDVFKELKVQHVNENIFYSPLSIISALSMVYLGARENTRAQIDK |
| | Ovalbumin-like | VVHFDKITGFEETIESQCSTSVSVHTSLKDMFTQITKPSDNYSLSFASRLYAEETY |
| | isoform X2 | PILPEYLQCVKELYKGGLETISFQTAADQARELINSWVESQTDGMIKNILQPGSVD |
| | [Gavia stellata] | PQTEMVLVNAIYFKGMWEKAFKDEDTQAVPFRMTEQESKPVQMMYQIGSFKVAVMA |
| | | SEKMKILELPYASGGMSMLVMLPDDVSGLEQLETAITFEKLMEWTSSNMMEERKMK |
| | | VYLPRMKMEEKYNLTSVLMALGMTDLFSSSANLSGISSAESLKMSEAVHEAFVEIY |
| | | EAGSEAVGSTGAGMEVTSVSEEFRADHPFLFLIKHNPTNSILFFGRCFSP |
| 24 | PREDICTED: | MGSIGAASTEFCFDVFKELKVQHVNENIFYSPLSIISALSMVYLGARENTRAQIDK |
| | Ovalbumin | VVHFDKITGFGEPIESQCGISVSVHTSLKDMITQITKPSDNYSLSFASRLYAEETY |
| | [Pelecanus | PILPEYLQCVKELYKGGLETISFQTAADQARELINSWVENQTNGMIKNILQPGSVD |
| | crispus] | PQTEMVLVNAVYFKGMWEKAFKDEDTQAVPFRMTEQESKPVQMMYQIGSFKVAVMA |
| | | SEKIKILELPYASGELSMLVLLPDDVSGLEQLETAITLDKLTEWTSSNAMEERKMK |
| | | VYLPRMKIEKKYNLTSVLIALGMTDLFSSSANLSGISSAESLKMSEAIHEAFLEIY |
| | | EAGSEVVGSTEAGMEVTSVSEEFRADHPFLFLIKHNPTNSILFFGRCLSP |
| 25 | PREDICTED: | MGSIGAASTEFCFDVFKELKVQHVNENIFYSPLTIISALSMVYLGARENTRAQIDK |
| | Ovalbumin-like | VVHFDKIPGFGDTTESQCGTSVSVHTSLKDMFTQITKPSDNYSVSFASRLYAEETY |
| | [Charadrius | PILPEFLECVKELYKGGLESISFQTAADQARELINSWVESQTNGMIKNILQPGSVD |
| | vociferus] | SQTEMVLVNAIYFKGMWEKAFKDEDTQTVPFRMTEQETKPVQMMYQIGTFKVAVMP |
| | | SEKMKILELPYASGELCMLVMLPDDVSGLEELESSITVEKLMEWTSSNMMEERKMK |
| | | VFLPRMKIEEKYNLTSVLMALGMTDLFSSSANLSGISSAEPLKMSEAVHEAFIEIY |
| | | EAGSEVVGSTGAGMEITSVSEEFRADHPFLFLIKHNPTNSILFFGRCVSP |
| 26 | PREDICTED: | MGSIGAVSTEFCFDVFKELKVQHVNENIFYSPLSIISALSMVYLGARENTRAQIDK |
| | Ovalbumin-like | VVHFDKITGSGETIEAQCGTSVSVHTSLKDMFTQITKPSENYSVGFASRLYADETY |
| | [Eurypyga | PIIPEYLQCVKELYKGGLEMISFQTAADQARELINSWVESQTNGMIKNILQPGSVD |
| | helias] | PQTEMILVNAIYFKGVWEKAFKDEDTQAVPFRMTEQESKPVQMMYQFGSFKVAAMA |
| | | AEKMKILELPYASGALSMLVLLPDDVSGLEQLESAITFEKLMEWTSSNMMEEKKIK |
| | | VYLPRMKMEEKYNFTSVLMALGMTDLFSSSANLSGISSADSLKMSEVVHEAFVEIY |
| | | EAGSEVVGSTGSGMEAASVSEEFRADHPFLFLIKHNPTNSILFFGRCFSP |
| 27 | PREDICTED: | MVSIGAASTEFCFDVFKELKVQHVNENIFYSPLSIISALSMVYLGARENTRAQIDK |
| | Ovalbumin-like | VVHFDKITGFEETIESQVQKKQCSTSVSVHTSLKDMFTQITKPSDNYSLSFASRLY |
| | isoform X1 | AEETYPILPEYLQCVKELYKGGLETISFQTAADQARELINSWVESQTDGMIKNILQ |
| | [Gavia stellata] | PGSVDPQTEMVLVNAIYFKGMWEKAFKDEDTQAVPFRMTEQESKPVQMMYQIGSFK |
| | | VAVMASEKMKILELPYASGGMSMLVMLPDDVSGLEQLETAITFEKLMEWTSSNMME |
| | | ERKMKVYLPRMKMEEKYNLTSVLMALGMTDLFSSSANLSGISSAESLKMSEAVHEA |
| | | FVEIYEAGSEAVGSTGAGMEVTSVSEEFRADHPFLFLIKHNPTNSILFFGRCFSP |
| 28 | PREDICTED: | MGSIGAASGEFCFDVFKELKVQHVNENIFYSPLSIISALSMVYLGARENTRAQIDK |
| | Ovalbumin-like | VVHFDKIIGFGESIESQCGTSVSVHTSLKDMFAQITKPSDNYSLSFASRLYAEETF |
| | [Egretta | PILPEYLQCVKELYKGGLETLSFQTAADQARELINSWVESQTNGMIKDILQPGSVD |
| | garzetta] | PQTEMVLVNAIYFKGVWEKAFKDEDTQTVPFRMTEQESKPVQMMYQIGSFKVAVVA |
| | | AEKIKILELPYASGALSMLVLLPDDVSSLEQLETAITFEKLTEWTSSNIMEERKIK |
| | | VYLPRMKIEEKYNLTSVLMDLGITDLFSSSANLSGISSAESLKVSEAIHEAIVDIY |
| | | EAGSEVVGSSGAGLEGTSVSEEFRADHPFLFLIKHNPTSSILFFGRCFSP |
| 29 | PREDICTED: | MGSIGAASTEFCFDVFKELKVQHVNENIFYSPLSIISALSMVYLGARENTRAQIDK |
| | Ovalbumin-like | VVHFDKITGSGEAIESQCGTSVSVHISLKDMFTQITKPSDNYSLSFASRLYAEETY |
| | [Balearica | PILPEYLQCVKELYKEGLATISFQTAADQAREFINSWVESQTNGMIKNILQPGSVD |
| | regulorum | PQTQMVLVNAIYFKGVWEKAFKDEDTQAVPFRMTKQESKPVQMMYQIGSFKVAVMA |
| | gibbericeps] | SEKMKILELPYASGQLSMLVMLPDDVSGLEQIENAITFEKLMEWTNPNMMEERKMK |
| | | VYLPRMKMEEKYNLTSVLMALGMTDLFSSSANLSGISSAESLKMSEAVHEAFVEIY |
| | | EAGSEVVGSTGAGIEVTSVSEEFRADHPFLFLIKHNPTNSILFFGRCFSP |
| 30 | PREDICTED: | MGSIGEASTEFCIDVFRELKVQHVNENIFYSPLSIISALSMVYLGARENTRAQIDQ |
| | Ovalbumin-like | VVHFDKITGFGDTVESQCGSSLSVHSSLKDIFAQITQPKDNYSLNFASRLYAEETY |
| | [Nestor | PILPEYLQCVKELYKGGLETISFQTAADQARELINSWVESQTNGMIKNILQPSSVD |
| | notabilis] | PQTEMVLVNAIYFKGVWEKAFKDEETQAVPFRITEQENRPVQIMYQFGSFKVAVVA |
| | | SEKIKILELPYASGQLSMLVLLPDEVSGLEQLENAITFEKLTEWTSSDIMEEKKIK |
| | | VFLPRMKIEEKYNLTSVLVALGIADLFSSSANLSGISSAESLKMSEAVHEAFVEIY |
| | | EAGSEVVGSSGAGIEAASDSEEFRADHPFLFLIKHKPTNSILFFGRCFSP |
| 31 | PREDICTED: | MGSIGAASTEFCFDIFNELKVQHVNENIFYSPLSIISALSMVYLGARENTKAQIDK |
| | Ovalbumin-like | VVHFDKITGFGESIESQCSTSASVHTSFKDMFTQITKPSDNYSLSFASRLYAEETY |
| | [Pygoscelis | PILPEYSQCVKELYKGGLESISFQTAADQARELINSWVESQTNGMIKNILQPGSVD |
| | adeliae] | PQTELVLVNAIYFKGTWEKAFKDKDTQAVPFRVTEQESKPVQMMYQIGSYKVAVIA |
| | | SEKMKILELPYASGELSMLVLLPDDVSGLEQLETAITFEKLMEWTSSNMMEERKVK |
| | | VYLPRMKIEEKYNLTSVLMALGMTDLFSPSANLSGISSAESLKMSEAIHEAFVEIY |
| | | EAGSEVVGSTEAGMEVTSVSEEFRADHPFLFLIKCNLTNSILFFGRCFSP |
| 32 | Ovalbumin-like | MGSISTASTEFCFDVFKELKVQHVNENIFYSPLSIISALSMVYLGARENTRAQIEK |
| | [Athene | VVHFDKITGFGESIESQCGTSVSVHTSLKDMLIQISKPSDNYSLSFASKLYAEETY |
| | cunicularia] | PILPEYLQCVKELYKGGLESINFQTAADQARQLINSWVESQTNGMIKDILQPSSVD |
| | | PQTEMVLVNAIYFKGIWEKAFKDEDTQEVPFRITEQESKPVQMMYQIGSFKVAVIA |
| | | SEKIKILELPYASGELSMLIVLPDDVSGLEQLETAITFEKLIEWTSPSIMEERKTK |
| | | VYLPRMKIEEKYNLTSVLMALGMTDLFSPSANLSGISSAESLKMSEAIHEAFVEIY |
| | | EAGSEVVGSAEAGMEATSVSEFRVDHPFLFLIKHNPANIILFFGRCVSP |
| 33 | PREDICTED: | MGSIGAASTEFCFDVFKELKVQHVNENIFYSPLTIISALSLVYLGARENTRAQIDK |
| | Ovalbumin-like | VFHFDKISGFGETTESQCGTSVSVHTSLKEMFTQITKPSDNYSVSFASRLYAEDTY |
| | [Calidris | PILPEYLQCVKELYKGGLETISFQTAADQAREVINSWVESQTNGMIKNILQPGSVD |
| | pugnax] | SQTEMVLVNAIYFKGMWEKAFKDEDTQTMPFRITEQERKPVQMMYQAGSFKVAVMA |
| | | SEKMKILELPYASGEFCMLIMLPDDVSGLEQLENSFSFEKLMEWTTSNMMEERKMK |
| | | VYIPRMKMEEKYNLTSVLMALGMTDLFSSSANLSGISSAETLKMSEAVHEAFMEIY |
| | | EAGSEVVGSTGSGAEVTGVYEEFRADHPFLFLVKHKPTNSILFFGRCVSP |
| 34 | PREDICTED: | MGSIGAASTEFCFDIFNELKVQHVNENIFYSPLSIISALSMVYLGARENTKAQIDK |
| | Ovalbumin | VVHFDKITGFGETIESQCSTSVSVHTSLKDTFTQITKPSDNYSLSFASRLYAEETY |
| | [Aptenodytes | PILPEYSQCVKELYKGGLETISFQTAADQARELINSWVESQTNGMIKNILQPGSVD |
| | forsteri] | PQTELVLVNAIYFKGTWEKAFKDKDTQAVPFRVTEQESKPVQMMYQIGSYKVAVIA |
| | | SEKMKILELPYASRELSMLVLLPDDVSGLEQLETAITFEKLMEWTSSNMMEERKVK |
| | | VYLPRMKIEEKYNLTSVLMALGMTDLFSPSANLSGISSAESLKMSEAVHEAFVEIY |
| | | EAGSEVVGSTGAGMEVTSVSEEFRADHPFLFLIKCNPTNSILFFGRCFSP |
| 35 | PREDICTED: | MGSISAASAEFCLDVFKELKVQHVNENIFYSPLSIISALSMVYLGARENTRAQIDK |
| | Ovalbumin-like | VVHFDKITGSGETIEFQCGTSANIHPSLKDMFTQITRLSDNYSLSFASRLYAEERY |
| | [Pterocles | PILPEYLQCVKELYKGGLETISFQTAADQARELINSWVESQTNGMIKNILQPGSVN |
| | gutturalis] | PQTEMVLVNAIYFKGLWEKAFKDEDTQTVPFRMTEQESKPVQMMYQVGSFKVAVMA |
| | | SDKIKILELPYASGELSMLVLLPDDVTGLEQLETSITFEKLMEWTSSNVMEERTMK |
| | | VYLPHMRMEEKYNLTSVLMALGVTDLFSSSANLSGISSAESLKMSEAVHEAFVEIY |
| | | ESGSQVVGSTGAGTEVTSVSEEFRVDHPFLFLIKHNPTNSILFFGRCFSP |
| 36 | Ovalbumin-like | MGSIGAASVEFCFDVFKELKVQHVNENIFYSPLSIISALSMVYLGARENTKAQIDK |
| | [Falco | VVHFDKIAGFGEAIESQCVTSASIHSLKDMFTQITKPSDNYSLSFASRLYAEEAYS |
| | peregrinus] | ILPEYLQCVKELYKGGLETISFQTAADQARDLINSWVESQTNGMIKNILQPGAVDL |
| | | ETEMVLVNAIYFKGMWEKAFKDEDTQTVPFRMTEQESKPVQMMYQVGSFKVAVMAS |
| | | DKIKILELPYASGQLSMVVVLPDDVSGLEQLEASITSEKLMEWTSSSIMEEKKIKV |
| | | YFPHMKIEEKYNLTSVLMALGMTDLFSSSANLSGISSAEKLKVSEAVHEAFVEISE |
| | | AGSEVVGSTEAGTEVTSVSEEFKADHPFLFLIKHNPTNSILFFGRCFSP |
| 37 | PREDICTED: | MGSIGAASSEFCFDIFKELKVQHVNENIFYSPLSIISALSMVYLGARENTRAQIDK |
| | Ovalbumin-like | VVPFDKITASGESIESQCSTSVSVHTSLKDIFTQITKSSDNHSLSFASRLYAEETY |
| | isoform X2 | PILPEYLQCVKELYEGGLETISFQTAADQARELINSWIESQTNGRIKNILQPGSVD |
| | [Phalacrocorax | PQTEMVLVNAIYFKGMWEKAFKDEDTQAVPFRMTEQESKPVQVMHQIGSFKVAVLA |
| | carbo] | SEKIKILELPYASGELSMLVLLPDDVSGLEQLETAITFEKLMEWTSPNIMEERKIK |
| | | VFLPRMKIEEKYNLTSVLMALGITDLFSPLANLSGISSAESLKMSEAIHEAFVEIS |
| | | EAGSEVIGSTEAEVEVTNDPEEFRADHPFLFLIKHNPTNSILFFGRCFSP |
| 38 | PREDICTED: | MGSIGAASTEFCFDVFKELKAQYVNENIFYSPMTIITALSMVYLGSKENTRAQIAK |
| | Ovalbumin-like | VAHFDKITGFGESIESQCGASASIQFSLKDLFTQITKPSGNHSLSVASRIYAEETY |
| | [Merops | PILPEYLECMKELYKGGLETINFQTAANQARELINSWVERQTSGMIKNILQPSSVD |
| | nubicus] | SQTEMVLVNAIYFRGLWEKAFKVEDTQATPFRITEQESKPVQMMHQIGSFKVAVVA |
| | | SEKIKILELPYASGRLTMLVVLPDDVSGLKQLETTITFEKLMEWTTSNIMEERKIK |
| | | VYLPRMKIEEKYNLTSVLMALGLTDLFSSSANLSGISSAESLKMSEAVHEAFVEIY |
| | | EAGSEVVASAEAGMDATSVSEEFRADHPFLFLIKDNTSNSILFFGRCFSP |
| 39 | PREDICTED: | MGSIGAASTEFCFDVFKELKGQHVNENIFFCPLSIVSALSMVYLGARENTRAQIVK |
| | Ovalbumin-like | VAHFDKIAGFAESIESQCGTSVSIHTSLKDMFTQITKPSDNYSLNFASRLYAEETY |
| | [Tauraco | PIIPEYLQCVKELYKGGLETISFQTAADQAREIINSWVESQTNGMIKNILRPSSVH |
| | erythrolophus] | PQTELVLVNAVYFKGTWEKAFKDEDTQAVPFRITEQESKPVQMMYQIGSFKVAAVT |
| | | SEKMKILEVPYASGELSMLVLLPDDVSGLEQLETAITAEKLIEWTSSTVMEERKLK |
| | | VYLPRMKIEEKYNLTTVLTALGVTDLFSSSANLSGISSAQGLKMSNAVHEAFVEIY |
| | | EAGSEVVGSKGEGTEVSSVSDEFKADHPFLFLIKHNPTNSIVFFGRCFSP |
| 40 | PREDICTED: | MGSIGAASTEFCFDVFKELKVHHVNENILYSPLAIISALSMVYLGAKENTRDQIDK |
| | Ovalbumin-like | VVHFDKITGIGESIESQCSTAVSVHTSLKDVFDQITRPSDNYSLAFASRLYAEKTY |
| | [Cuculus | PILPEYLQCVKELYKGGLETIDFQTAADQARQLINSWVEDETNGMIKNILRPSSVN |
| | canorus] | PQTKIILVNAIYFKGMWEKAFKDEDTQEVPFRITEQETKSVQMMYQIGSFKVAEVV |
| | | SDKMKILELPYASGKLSMLVLLPDDVYGLEQLETVITVEKLKEWTSSIVMEERITK |
| | | VYLPRMKIMEKYNLTSVLTAFGITDLFSPSANLSGISSTESLKVSEAVHEAFVEIH |
| | | EAGSEVVGSAGAGIEATSVSEEFKADHPFLFLIKHNPTNSILFFGRCFSP |
| 41 | Ovalbumin | MGSIGAASTEFCLDVFKELKVQHVNENIFYSPLSIISALSMVYLGARENTRAQIDK |
| | [Antrostomus | VVHFDKITGFEDSIESQCGTSVSVHTSLKDMFTQITKPSDNYSVGFASRLYAAETY |
| | carolinensis] | QILPEYSQCVKELYKGGLETINFQKAADQATELINSWVESQTNGMIKNILQPSSVD |
| | | PQTQIFLVNAIYFKGMWQRAFKEEDTQAVPFRISEKESKPVQMMYQIGSFKVAVIP |
| | | SEKIKILELPYASGLLSMLVILPDDVSGLEQLENAITLEKLMQWTSSNMMEERKIK |
| | | VYLPRMRMEEKYNLTSVFMALGITDLFSSSANLSGISSAESLKMSDAVHEASVEIH |
| | | EAGSEVVGSTGSGTEASSVSEEFRADHPYLFLIKHNPTDSIVFFGRCFSP |
| 42 | PREDICTED: | MGSIGAASTEFCFDVFKELKFQHVDENIFYSPLTIISALSMVYLGARENTRAQIDK |
| | Ovalbumin-like | VVHFDKIAGFEETVESQCGTSVSVHTSLKDMFAQITKPSDNYSLSFASRLYAEETY |
| | [Opisthocomus | PILPEYLQCVKELYKGGLETISFQTAADQARDLINSWVESQTNGMIKNILQPSSVG |
| | hoazin] | PQTELILVNAIYFKGMWQKAFKDEDTQEVPFRMTEQQSKPVQMMYQTGSFKVAVVA |
| | | SEKMKILALPYASGQLSLLVMLPDDVSGLKQLESAITSEKLIEWTSPSMMEERKIK |
| | | VYLPRMKIEEKYNLTSVLMALGITDLFSPSANLSGISSAESLKMSQAVHEAFVEIY |
| | | EAGSEVVGSTGAGMEDSSDSEEFRVDHPFLFFIKHNPTNSILFFGRCFSP |
| 43 | PREDICTED: | MGSIGPLSVEFCCDVFKELRIQHPRENIFYSPVTIISALSMVYLGARDNTKAQIEK |
| | Ovalbumin-like | AVHFDKIPGFGESIESQCGTSLSIHTSLKDIFTQITKPSDNYTVGIASRLYAEEKY |
| | [Lepidothrix | PILPEYLQCIKELYKGGLEPINFQTAAEQARELINSWVESQTNGMIKNILQPSSVN |
| | coronata] | PETDMVLVNAIYFKGLWEKAFKDEDIQTVPFRITEQESKPVQMMFQIGSFRVAEIT |
| | | SEKIRILELPYASGQLSLWVLLPDDISGLEQLETAITFENLKEWTSSTKMEERKIK |
| | | VYLPRMKIEEKYNLTSVLTSLGITDLFSSSANLSGISSAESLKVSSAFHEASVEIY |
| | | EAGSKVVGSTGAEVEDTSVSEEFRADHPFLFLIKHNPSNSIFFFGRCFSP |
| 44 | PREDICTED: | MGSIGTASAEFCFDVFKELKVHHVNENIFYSPLSIISALSMVYLGARENTKTQMEK |
| | Ovalbumin | VIHFDKITGLGESMESQCGTGVSIHTALKDMLSEITKPSDNYSLSLASRLYAEQTY |
| | [Struthio | AILPEYLQCIKELYKESLETVSFQTAADQARELINSWIESQTNGVIKNFLQPGSVD |
| | camelus | SQTELVLVNAIYFKGMWEKAFKDEDTQEVPFRITEQESRPVQMMYQAGSFKVATVA |
| | australis] | AEKIKILELPYASGELSMLVLLPDDISGLEQLETTISFEKLTEWTSSNMMEDRNMK |
| | | VYLPRMKIEEKYNLTSVLIALGMTDLFSPAANLSGISAAESLKMSEAIHAAYVEIY |
| | | EADSEIVSSAGVQVEVTSDSEEFRVDHPFLFLIKHNPTNSVLFFGRCISP |
| 45 | PREDICTED: | MGSIGAVSTEFSCDVFKELRIHHVQENIFYSPVTIISALSMIYLGARDSTKAQIEK |
| | Ovalbumin-like | AVHFDKIPGFGESIESQCGTSLSIHTSIKDMFTKITKASDNYSIGIASRLYAEEKY |
| | [Acanthisitta | PILPEYLQCVKELYKGGLESISFQTAAEQAREIINSWVESQTNGMIKNILQPSSVD |
| | chloris] | PQTDIVLVNAIYFKGLWEKAFRDEDTQTVPFKITEQESKPVQMMYQIGSFKVAEIT |
| | | SEKIKILEVPYASGQLSLWVLLPDDISGLEKLETAITFENLKEWTSSTKMEERKIK |
| | | VYLPRMKIEEKYNLTSVLTALGITDLFSSSANLSGISSAESLKVSEAFHEAIVEIS |
| | | EAGSKVVGSVGAGVDDTSVSEEFRADHPFLFLIKHNPTSSIFFFGRCFSP |
| 46 | PREDICTED: | MGSIGAASTEFCFDVFKELKVQHVNENIFYSPLSIISALSMVYLGARENTRAQIDK |
| | Ovalbumin-like | VVHFDKIAGFGESTESQCGTSVSAHTSLKDMSNQITKLSDNYSLSFASRLYAEETY |
| | [Tyto alba] | PILPEYSQCVKELYKGGLESISFQTAAYQARELINAWVESQTNGMIKDILQPGSVD |
| | | SQTKMVLVNAIYFKGIWEKAFKDEDTQEVPFRMTEQETKPVQMMYQIGSFKVAVIA |
| | | AEKIKILELPYASGQLSMLVILPDDVSGLEQLETAITFEKLTEWTSASVMEERKIK |
| | | VYLPRMSIEEKYNLTSVLIALGVTDLFSSSANLSGISSAESLRMSEAIHEAFVETY |
| | | EAGSTESGTEVTSASEEFRVDHPFLFLIKHKPTNSILFFGRCFSP |
| 47 | PREDICTED: | MGSIGAASSEFCFDIFKELKVQHVNENIFYSPLSIISALSMVYLGARENTRAQIDK |
| | Ovalbumin-like | VVPFDKITASGESIESQVQKIQCSTSVSVHTSLKDIFTQITKSSDNHSLSFASRLY |
| | isoform X1 | AEETYPILPEYLQCVKELYEGGLETISFQTAADQARELINSWIESQTNGRIKNILQ |
| | [Phalacrocorax | PGSVDPQTEMVLVNAIYFKGMWEKAFKDEDTQAVPFRMTEQESKPVQVMHQIGSFK |
| | carbo] | VAVLASEKIKILELPYASGELSMLVLLPDDVSGLEQLETAITFEKLMEWTSPNIME |
| | | ERKIKVFLPRMKIEEKYNLTSVLMALGITDLFSPLANLSGISSAESLKMSEAIHEA |
| | | FVEISEAGSEVIGSTEAEVEVTNDPEEFRADHPFLFLIKHNPTNSILFFGRCFSP |
| 48 | Ovalbumin-like | MGSIGPLSVEFCCDVFKELRIQHARENIFYSPVTIISALSMVYLGARDNTKAQIEK |
| | [Pipra filicauda] | AVHFDKIPGFGESIESQCGTSLSIHTSLKDIFTQITKPSDNYTVGIASRLYAEEKY |
| | | PILPEYLQCIKELYKGGLEPISFQTAAEQARELINSWVESQTNGIIKNILQPSSVN |
| | | PETDMVLVNAIYFKGLWEKAFKDEGTQTVPFRITEQESKPVQMMFQIGSFRVAEIA |
| | | SEKIRILELPYASGQLSLWVLLPDDISGLEQLETAITFENLKEWTSSTKMEERKIK |
| | | VYLPRMKIEEKYNLTSVLTSLGITDLFSSSANLSGISSAERLKVSSAFHEASMEIN |
| | | EAGSKVVGAGVDDTSVSEEFRVDRPFLFLIKHNPSNSIFFFGRCFSP |
| 49 | Ovalbumin | MGSIGAASTEFCFDMFKELKVHHVNENIIYSPLSIISILSMVFLGARENTKTQMEK |
| | [Dromaius | VIHFDKITGFGESLESQCGTSVSVHASLKDILSEITKPSDNYSLSLASKLYAEETY |
| | novaehollandiae] | PVLPEYLQCIKELYKGSLETVSFQTAADQARELINSWVETQTNGVIKNFLQPGSVD |
| | | PQTEMVLVDAIYFKGTWEKAFKDEDTQEVPFRITEQESKPVQMMYQAGSFKVATVA |
| | | AEKMKILELPYASGELSMFVLLPDDISGLEQLETTISIEKLSEWTSSNMMEDRKMK |
| | | VYLPHMKIEEKYNLTSVLVALGMTDLFSPSANLSGISTAQTLKMSEAIHGAYVEIY |
| | | EAGSEMATSTGVLVEAASVSEEFRVDHPFLFLIKHNPSNSILFFGRCIFP |
| 50 | Chain A, | MGSIGAASTEFCFDMFKELKVHHVNENIIYSPLSIISILSMVFLGARENTKTQMEK |
| | Ovalbumin | VIHFDKITGFGESLESQCGTSVSVHASLKDILSEITKPSDNYSLSLASKLYAEETY |
| | | PVLPEYLQCIKELYKGSLETVSFQTAADQARELINSWVETQTNGVIKNFLQPGSVD |
| | | PQTEMVLVDAIYFKGTWEKAFKDEDTQEVPFRITEQESKPVQMMYQAGSFKVATVA |
| | | AEKMKILELPYASGELSMFVLLPDDISGLEQLETTISIEKLSEWTSSNMMEDRKMK |
| | | VYLPHMKIEEKYNLTSVLVALGMTDLFSPSANLSGISTAQTLKMSEAIHGAYVEIY |
| | | EAGSEMATSTGVLVEAASVSEEFRVDHPFLFLIKHNPSNSILFFGRCIFPHHHHHH |
| 51 | Ovalbumin-like | MGSIGPLSVEFCCDVFKELRIQHARENIFYSPVTIISALSMVYLGARDNTKAQIEK |
| | [Corapipo | AVHFDKIPGFGESIESQCGTSLSIHTSLKDIFTQITKPSDNYTVGIASRLYAEEKY |
| | altera] | PILPEYLQCIKELYKGGLEPISFQTAAEQARELINSWVESQTNGMIKNILQPSAVN |
| | | PETDMVLVNAIYFKGLWEKAFKDEGTQTVPFRITEQESKPVQMMFQIGSFRVAEIT |
| | | SEKIRILELPYASGQLSLWVLLPDDISGLEQLETAITFENLKEWTSSTKMEERKIK |
| | | VYLPRMKIEEKYNLTSVLTSLGITDLFSSSANLSGISSAERLKVSSAFHEASMEIY |
| | | EAGSKVVGSTGAGVDDTSVSEEFRVDRPFLFLIKHNPSNSIFFFGRCFSP |
| 52 | Ovalbumin-like | MEDQRGNTGFTMGSIGAASTEFCIDVFRELRVQHVNENIFYSPLTIISALSMVYLG |
| | protein | ARENTRAQIDQVVHFDKIAGFGDTVESQCGSSPSVHNSLKTVXAQITQPRDNYSLN |
| | [Amazona | LASRLYAEESYPILPEYLQCVKELYNGGLETVSFQTAADQARELINSWVESQTNGI |
| | aestiva] | IKNILQPSSVDPQTEMVLVNAIYFKGLWEKAFKDEETQAVPFRITEQENRPVQMMY |
| | | QFGSFKVAXVASEKIKILELPYASGQLSMLVLLPDEVSGLEQNAITFEKLTEWTSS |
| | | DLMEERKIKVFFPRVKIEEKYNLTAVLVSLGITDLFSSSANLSGISSAENLKMSEA |
| | | VHEAXVEIYEAGSEVAGSSGAGIEVASDSEEFRVDHPFLFLIXHNPTNSILFFGRC |
| | | FSP |
| 53 | PREDICTED: | MGSIGAASTEFCIDVFRELRVQHVNENIFYSPLSIISALSMVYLGARENTRAQIDE |
| | Ovalbumin-like | VFHFDKIAGFGDTVDPQCGASLSVHKSLQNVFAQITQPKDNYSLNLASRLYAEESY |
| | [Melopsittacus | PILPEYLQCVKELYNEGLETVSFQTGADQARELINSWVENQTNGVIKNILQPSSVD |
| | undulatus] | PQTEMVLVNAIYFKGLWQKAFKDEETQAVPFRITEQENRPVQMMYQFGSFKVAVVA |
| | | SEKVKILELPYASGQLSMWVLLPDEVSGLEQLENAITFEKLTEWTSSDLTEERKIK |
| | | VFLPRVKIEEKYNLTAVLMALGVTDLFSSSANFSGISAAENLKMSEAVHEAFVEIY |
| | | EAGSEVVGSSGAGIEAPSDSEEFRADHPFLFLIKHNPTNSILFFGRCFSP |
| 54 | Ovalbumin-like | MGSIGPLSVEFCCDVFKELRIQHARDNIFYSPVTIISALSMVYLGARDNTKAQIEK |
| | [Neopelma | AVHFDKIPGFGESIESQCGTSLSVHTSLKDIFTQITKPRENYTVGIASRLYAEEKY |
| | chrysocephalum] | PILPEYLQCIKELYKGGLEPISFQTAAEQARELINSWVESQTNGMIKNILQPSSVN |
| | | PETDMVLVNAIYFKGLWKKAFKDEGTQTVPFRITEQESKPVQMMFQIGSFRVAEIT |
| | | SEKIRILELPYASGQLSLWVLLPDDISGLEQLESAITFENLKEWTSSTKMEERKIK |
| | | VYLPRMKIEEKYNLTSVLTSLGITDLFSSSANLSGISSAEKLKVSSAFHEASMEIY |
| | | EAGNKVVGSTGAGVDDTSVSEEFRVDRPFLFLIKHNPSNSIFFFGRCFSP |
| 55 | PREDICTED: | MGSIGAASAEFCVDVFKELKDQHVNNIVFSPLMIISALSMVNIGAREDTRAQIDKV |
| | Ovalbumin-like | VHFDKITGYGESIESQCGTSIGIYFSLKDAFTQITKPSDNYSLSFASKLYAEETYP |
| | [Buceros | ILPEYLKCVKELYKGGLETISFQTAADQARELINSWVESQTNGMIKNILQPSSVDP |
| | rhinoceros | QTEMVLVNAIYFKGLWEKAFKDEDTQAVPFRITEQESKPVQMMYQIGSFKVAVIAS |
| | silvestris] | EKIKILELPYASGQLSLLVLLPDDVSGLEQLESAITSEKLLEWTNPNIMEERKTKV |
| | | YLPRMKIEEKYNLTSVLVALGITDLFSSSANLSGISSAEGLKLSDAVHEAFVEIYE |
| | | AGREVVGSSEAGVEDSSVSEEFKADRPFIFLIKHNPTNGILYFGRYISP |
| 56 | PREDICTED: | MGSIGAANTDFCFDVFKELKVHHANENIFYSPLSIVSALAMVYLGARENTRAQIDK |
| | Ovalbumin-like | ALHFDKILGFGETVESQCDTSVSVHTSLKDMLIQITKPSDNYSFSFASKIYTEETY |
| | [Cariama | PILPEYLQCVKELYKGGVETISFQTAADQAREVINSWVESHTNGMIKNILQPGSVD |
| | cristata] | PQTKMVLVNAVYFKGIWEKAFKEEDTQEMPFRINEQESKPVQMMYQIGSFKLTVAA |
| | | SENLKILEFPYASGQLSMMVILPDEVSGLKQLETSITSEKLIKWTSSNTMEERKIR |
| | | VYLPRMKIEEKYNLKSVLMALGITDLFSSSANLSGISSAESLKMSEAVHEAFVEIY |
| | | EAGSEVTSSTGTEMEAENVSEEFKADHPFLFLIKHNPTDSIVFFGRCMSP |
| 57 | Ovalbumin | MGSIGPLSVEFCCDVFKELRIQHARENIFYSPVTIISALSMVYLGARDNTKAQIEK |
| | [Manacus | AVHFDKIPGFGESIESQCGTSLSIHTSLKDIFTQITKPSDNYTVGIASRLYAEEKY |
| | vitellinus] | PILPEYLQCIKELYKGGLEPISFQTAAEQARELINSWVESQTNGMIKNILQPSSVN |
| | | PETDMVLVNAIYFKGLWEKAFKDESTQTVPFRITEQESKPVQMMFQIGSFRVAEIA |
| | | SEKIRILELPYASGQLSLWVLLPDDISGLEQLETAITFENLKEWTSSTKMEERKIK |
| | | VYLPRMKIEEKYNLTSVLTSLGITDLFSSSANLSGISSAERLKVSSAFHEASMEIY |
| | | EAGSRVVEAGVDDTSVSEEFRVDRPFLFLIKHNPSNSIFFFGRCFSP |
| 58 | Ovalbumin-like | MGSIGPVSTEFCCDIFKELRIQHARENIIYSPVTIISALSMVYLGARDNTKAQIEK |
| | [Empidonax | AVHFDKIPGFGESIESQCGTSLSIHTSLKDILTQITKPSDNYTVGIASRLYAEEKY |
| | traillii] | PILSEYLQCIKELYKGGLEPISFQTAAEQARELINSWVESQTNGMIKNILQPSSVN |
| | | PETDMVLVNAIYFKGLWEKAFKDEGTQTVPFRITEQESKPVQMMFQIGSFKVAEIT |
| | | SEKIRILELPYASGKLSLWVLLPDDISGLEQLETAITFENLKEWTSSTRMEERKIK |
| | | VYLPRMKIEEKYNLTSVLTSLGITDLFSSSANLSGISSAERLKVSSAFHEVFVEIY |
| | | EAGSKVEGSTGAGVDDTSVSEEFRADHPFLFLVKHNPSNSIIFFGRCYLP |
| 59 | PREDICTED: | MGSTGAASMEFCFALFRELKVQHVNENIFFSPVTIISALSMVYLGARENTRAQLDK |
| | Ovalbumin-like | VAPFDKITGFGETIGSQCSTSASSHTSLKDVFTQITKASDNYSLSFASRLYAEETY |
| | [Leptosomus | PILPEYLQCVKELYKGGLESISFQTAADQARELINSWVESQTNGMIKDILRPSSVD |
| | discolor] | PQTKIILITAIYFKGMWEKAFKEEDTQAVPFRMTEQESKPVQMMYQIGSFKVAVIP |
| | | SEKLKILELPYASGQLSMLVILPDDVSGLEQLETAITTEKLKEWTSPSMMKERKMK |
| | | VYFPRMRIEEKYNLTSVLMALGITDLFSPSANLSGISSAESLKVSEAVHEASVDID |
| | | EAGSEVIGSTGVGTEVTSVSEEIRADHPFLFLIKHKPTNSILFFGRCFSP |
| 60 | Hypothetical | MEHAQLTQLVNSNMTSNTCHEADEFENIDFRMDSISVTNTKFCFDVFNEMKVHHVN |
| | protein | ENILYSPLSILTALAMVYLGARGNTESQMKKALHFDSITGAGSTTDSQCGSSEYIH |
| | H355_008077 | NLFKEFLTEITRTNATYSLEIADKLYVDKTFTVLPEYINCARKFYTGGVEEVNFKT |
| | [Colinus | AAEEARQLINSWVEKETNGQIKDLLVPSSVDFGTMMVFINTIYFKGIWKTAFNTED |
| | virginianus] | TREMPFSMTKQESKPVQMMCLNDTFNMATLPAEKMRILELPYASGELSMLVLLPDE |
| | | VSGLEQIEKAINFEKLREWTSTNAMEKKSMKVYLPRMKIEEKYNLTSTLMALGMTD |
| | | LFSRSANLTGISSVENLMISDAVHGAFMEVNEEGTEAAGSTGAIGNIKHSVEFEEF |
| | | RADHPFLFLIRYNPTNVILFFDNSEFTMGSIGAVSTEFCFDVFKELRVHHANENIF |
| | | YSPFTVISALAMVYLGAKDSTRTQINKVVRFDKLPGFGDSIEAQCGTSANVHSSLR |
| | | DILNQITKPNDIYSFSLASRLYADETYTILPEYLQCVKELYRGGLESINFQTAADQ |
| | | ARELINSWVESQTSGIIRNVLQPSSVDSQTAMVLVNAIYFKGLWEKGFKDEDTQAM |
| | | PFRVTEQENKSVQMMYQIGTFKVASVASEKMKILELPFASGTMSMWVLLPDEVSGL |
| | | EQLETTISIEKLTEWTSSSVMEERKIKVFLPRMKMEEKYNLTSVLMAMGMTDLFSS |
| | | SANLSGISSTLQKKGFRSQELGDKYAKPMLESPALTPQVTAWDNSWIVAHPAAIEP |
| | | DLCYQIMEQKWKPFDWPDFRLPMRVSCRFRTMEALNKANTSFALDFFKHECQEDDD |
| | | ENILFSPFSISSALATVYLGAKGNTADQMAKTEIGKSGNIHAGFKALDLEINQPTK |
| | | NYLLNSVNQLYGEKSLPFSKEYLQLAKKYYSAEPQSVDFLGKANEIRREINSRVEH |
| | | QTEGKIKNLLPPGSIDSLTRLVLVNALYFKGNWATKFEAEDTRHRPFRINMHTTKQ |
| | | VPMMYLRDKFNWTYVESVQTDVLELPYVNNDLSMFILLPRDITGLQKLINELTFEK |
| | | LSAWTSPELMEKMKMEVYLPRFTVEKKYDMKSTLSKMGIEDAFTKVDSCGVTNVDE |
| | | ITTHIVSSKCLELKHIQINKKLKCNKAVAMEQVSASIGNFTIDLFNKLNETSRDKN |
| | | IFFSPWSVSSALALTSLAAKGNTAREMAEDPENEQAENIHSGFKELMTALNKPRNT |
| | | YSLKSANRIYVEKNYPLLPTYIQLSKKYYKAEPYKVNFKTAPEQSRKEINNWVEKQ |
| | | TERKIKNFLSSDDVKNSTKSILVNAIYFKAEWEEKFQAGNTDMQPFRMSKNKSKLV |
| | | KMMYMRHTFPVLIMEKLNFKMIELPYVKRELSMFILLPDDIKDSTTGLEQLERELT |
| | | YEKLSEWADSKKMSVTLVDLHLPKFSMEDRYDLKDALKSMGMASAFNSNADFSGMT |
| | | GFQAVPMESLSASTNSFTLDLYKKLDETSKGQNIFFASWSIATALAMVHLGAKGDT |
| | | ATQVAKGPEYEETENIHSGFKELLSAINKPRNTYLMKSANRLFGDKTYPLLPKFLE |
| | | LVARYYQAKPQAVNFKTDAEQARAQINSWVENETESKIQNLLPAGSIDSHTVLVLV |
| | | NAIYFKGNWEKRFLEKDTSKMPFRLSKTETKPVQMMFLKDTFLIHHERTMKFKIIE |
| | | LPYVGNELSAFVLLPDDISDNTTGLELVERELTYEKLAEWSNSASMMKAKVELYLP |
| | | KLKMEENYDLKSVLSDMGIRSAFDPAQADFTRMSEKKDLFISKVIHKAFVEVNEED |
| | | RIVQLASGRLTGRCRTLANKELSEKNRTKNLFFSPFSISSALSMILLGSKGNTEAQ |
| | | IAKVLSLSKAEDAHNGYQSLLSEINNPDTKYILRTANRLYGEKTFEFLSSFIDSSQ |
| | | KFYHAGLEQTDFKNASEDSRKQINGWVEEKTEGKIQKLLSEGIINSMTKLVLVNAI |
| | | YFKGNWQEKFDKETTKEMPFKINKNETKPVQMMFRKGKYNMTYIGDLETTVLEIPY |
| | | VDNELSMIILLPDSIQDESTGLEKLERELTYEKLMDWINPNMMDSTEVRVSLPRFK |
| | | LEENYELKPTLSTMGMPDAFDLRTADFSGISSGNELVLSEVVHKSFVEVNEEGTEA |
| | | AAATAGIMLLRCAMIVANFTADHPFLFFIRHNKTNSILFCGRFCSP |
| 61 | PREDICTED: | MGSIGTASTEFCFDMFKEMKVQHANQNIIFSPLTIISALSMVYLGARDNTKAQMEK |
| | Ovalbumin | VIHFDKITGFGESVESQCGTSVSIHTSLKDMLSEITKPSDNYSLSLASRLYAEETY |
| | isoform X2 | PILPEYLQCMKELYKGGLETVSFQTAADQARELINSWVESQTNGVIKNFLQPGSVD |
| | [Apteryx | PQTEMVLVNAIYFKGMWEKAFKDEDTQEVPFRITEQESKPVQMMYQVGSFKVATVA |
| | australis | AEKMKILEIPYTHRELSMFVLLPDDISGLEQLETTISFEKLTEWTSSNMMEERKVK |
| | mantelli] | VYLPHMKIEEKYNLTSVLMALGMTDLFSPSANLSGISTAQTLMMSEAIHGAYVEIY |
| | | EAGREMASSTGVQVEVTSVLEEVRADKPFLFFIRHNPTNSMVVFGRYMSP |
| 62 | Hypothetical | MTSNTCHEADEFENIDFRMDSISVTNTKFCFDVFNEMKVHHVNENILYSPLSILTA |
| | protein | LAMVYLGARGNTESQMKKALHFDSITGGGSTTDSQCGSSEYIHNLFKEFLTEITRT |
| | ASZ78_006007 | NATYSLEIADKLYVDKTFTVLPEYINCARKFYTGGVEEVNFKTAAEEARQLMNSWV |
| | [Callipepla | EKETNGQIKDLLVPSSVDFGTMMVFINTIYFKGIWKTAFNTEDTREMPFSMTKQES |
| | squamata] | KPVQMMCLNDTFNMVTLPAEKMRILELPYASGELSMLVLLPDEVSGLERIEKAINF |
| | | EKLREWTSTNAMEKKSMKVYLPRMKIEEKYNLTSTLMALGMTDLFSRSANLTGISS |
| | | VDNLMISDAVHGAFMEVNEEGTEAAGSTGAIGNIKHSVEFEEFRADHPFLFLIRYN |
| | | PTNVILFFDNSEFTMGSIGAVSTEFCFDVFKELRVHHANENIFYSPFTIISALAMV |
| | | YLGAKDSTRTQINKVVRFDKLPGFGDSIEAQCGTSANVHSSLRDILNQITKPNDIY |
| | | SFSLASRLYADETYTILPEYLQCVKELYRGGLESINFQTAADQARELINSWVESQT |
| | | SGIIRNVLQPSSVDSQTAMVLVNAIYFKGLWEKGFKDEDTQAIPFRVTEQENKSVQ |
| | | MMYQIGTFKVASVASEKMKILELPFASGTMSMWVLLPDEVSGLEQLETTISIEKLT |
| | | EWTSSSVMEERKIKVFLPRMKMEEKYNLTSVLMAMGMTDLFSSSANLSGISSTLQK |
| | | KGFRSQELGDKYAKPMLESPALTPQATAWDNSWIVAHPPAIEPDLYYQIMEQKWKP |
| | | FDWPDFRLPMRVSCRFRTMEALNKANTSFALDFFKHECQEDDSENILFSPFSISSA |
| | | LATVYLGAKGNTADQMAKVLHFNEAEGARNVTTTIRMQVYSRTDQQRLNRRACFQK |
| | | TEIGKSGNIHAGFKGLNLEINQPTKNYLLNSVNQLYGEKSLPFSKEYLQLAKKYYS |
| | | AEPQSVDFVGTANEIRREINSRVEHQTEGKIKNLLPPGSIDSLTRLVLVNALYFKG |
| | | NWATKFEAEDTRHRPFRINTHTTKQVPMMYLSDKFNWTYVESVQTDVLELPYVNND |
| | | LSMFILLPRDITGLQKLINELTFEKLSAWTSPELMEKMKMEVYLPRFTVEKKYDMK |
| | | STLSKMGIEDAFTKVDNCGVTNVDEITIHVVPSKCLELKHIQINKELKCNKAVAME |
| | | QVSASIGNFTIDLFNKLNETSRDKNIFFSPWSVSSALALTSLAAKGNTAREMAEDP |
| | | ENEQAENIHSGFNELLTALNKPRNTYSLKSANRIYVEKNYPLLPTYIQLSKKYYKA |
| | | EPHKVNFKTAPEQSRKEINNWVEKQTERKIKNFLSSDDVKNSTKLILVNAIYFKAE |
| | | WEEKFQAGNTDMQPFRMSKNKSKLVKMMYMRHTFPVLIMEKLNFKMIELPYVKREL |
| | | SMFILLPDDIKDSTTGLEQLERELTYEKLSEWADSKKMSVTLVDLHLPKFSMEDRY |
| | | DLKDALRSMGMASAFNSNADFSGMTGERDLVISKVCHQSFVAVDEKGTEAAAATAV |
| | | IAEAVPMESLSASTNSFTLDLYKKLDETSKGQNIFFASWSIATALTMVHLGAKGDT |
| | | ATQVAKGPEYEETENIHSGFKELLSALNKPRNTYSMKSANRLFGDKTYPLLPTKTK |
| | | PVQMMFLKDTFLIHHERTMKFKIIELPYMGNELSAFVLLPDDISDNTTGLELVERE |
| | | LTYEKLAEWSNSASMMKVKVELYLPKLKMEENYDLKSALSDMGIRSAFDPAQADFT |
| | | RMSEKKDLFISKVIHKAFVEVNEEDRIVQLASGRLTGNTEAQIAKVLSLSKAEDAH |
| | | NGYQSLLSEINNPDTKYILRTANRLYGEKTFEFLSSFIDSSQKFYHAGLEQTDFKN |
| | | ASEDSRKQINGWVEEKTEGKIQKLLSEGIINSMTKLVLVNAIYFKGNWQEKFDKET |
| | | TKEMPFKINKNETKPVQMMFRKGKYNMTYIGDLETTVLEIPYVDNELSMIILLPDS |
| | | IQDESTGLEKLERELTYEKLMDWINPNMMDSTEVRVSLPRFKLEENYELKPTLSTM |
| | | GMPDAFDLRTADESGISSGNELVLSEVVHKSFVEVNEEGTEAAAATAGIMLLRCAM |
| | | IVANFTADHPFLFFIRHNKTNSILFCGRFCSP |
| 63 | PREDICTED: | MASIGAASTEFCFDVFKELKTQHVKENIFYSPMAIISALSMVYIGARENTRAEIDK |
| | Ovalbumin-like | VVHFDKITGFGNAVESQCGPSVSVHSSLKDLITQISKRSDNYSLSYASRIYAEETY |
| | [Mesitornis | PILPEYLQCVKEVYKGGLESISFQTAADQARENINAWVESQTNGMIKNILQPSSVN |
| | unicolor] | PQTEMVLVNAIYLKGMWEKAFKDEDTQTMPFRVTQQESKPVQMMYQIGSFKVAVIA |
| | | SEKMKILELPYTSGQLSMLVLLPDDVSGLEQVESAITAEKLMEWTSPSIMEERTMK |
| | | VYLPRMKMVEKYNLTSVLMALGMTDLFTSVANLSGISSAQGLKMSQAIHEAFVEIY |
| | | EAGSEAVGSTGVGMEITSVSEEFKADLSFLFLIRHNPTNSIIFFGRCISP |
| 64 | Ovalbumin, | MGSIGAASTEFCFDVFRELRVQHVNENIFYSPFSIISALAMVYLGARDNTRTQIDK |
| | partial [Anas | ISQFQALSDEHLVLCIQQLGEFFVCTNRERREVTRYSEQTEDKTQDQNTGQIHKIV |
| | platyrhynchos] | DTCMLRQDILTQITKPSDNFSLSFASRLYAEETYAILPEYLQCVKELYKGGLESIS |
| | | FQTAADQARELINSWVESQTNGIIKNILQPSSVDSQTTMVLVNAIYFKGMWEKAFK |
| | | DEDTQAMPFRMTEQESKPVQMMYQVGSFKVAMVTSEKMKILELPFASGMMSMFVLL |
| | | PDEVSGLEQLESTISFEKLTEWTSSTMMEERRMKVYLPRMKMEEKYNLTSVFMALG |
| | | MTDLFSSSANMSGISSTVSLKMSEAVHAACVEIFEAGRDVVGSAEAGMDVTSVSEE |
| | | FRADHPFLFFIKHNPTNSILFFGRWMSP |
| 65 | PREDICTED: | MGSIGAASAEFCLDIFKELKVQHVNENIIFSPMTIISALSLVYLGAKEDTRAQIEK |
| | Ovalbumin-like | VVPFDKIPGFGEIVESQCPKSASVHSSIQDIFNQIIKRSDNYSLSLASRLYAEESY |
| | [Chaetura | PIRPEYLQCVKELDKEGLETISFQTAADQARQLINSWVESQTNGMIKNILQPSSVN |
| | pelagica] | SQTEMVLVNAIYFRGLWQKAFKDEDTQAVPFRITEQESKPVQMMQQIGSFKVAEIA |
| | | SEKMKILELPYASGQLSMLVLLPDDVSGLEKLESSITVEKLIEWTSSNLTEERNVK |
| | | VYLPRLKIEEKYNLTSVLAALGITDLFSSSANLSGISTAESLKLSRAVHESFVEIQ |
| | | EAGHEVEGPKEAGIEVTSALDEFRVDRPFLFVTKHNPTNSILFLGRCLSP |
| 66 | PREDICTED: | MGSISAASGEFCLDIFKELKVQHVNENIFYSPMVIVSALSLVYLGARENTRAQIDK |
| | Ovalbumin-like | VIPFDKITGSSEAVESQCGTPVGAHISLKDVFAQIAKRSDNYSLSFVNRLYAEETY |
| | [Apaloderma | PILPEYLQCVKELYKGGLETISFQTAADQAREIINSWVESQTDGKIKNILQPSSVD |
| | vittatum] | PQTKMVLVSAIYFKGLWEKSFKDEDTQAVPFRVTEQESKPVQMMYQIGSFKVAAIA |
| | | AEKIKILELPYASEQLSMLVLLPDDVSGLEQLEKKISYEKLTEWTSSSVMEEKKIK |
| | | VYLPRMKIEEKYNLTSILMSLGITDLFSSSANLSGISSTKSLKMSEAVHEASVEIY |
| | | EAGSEASGITGDGMEATSVFGEFKVDHPFLFMIKHKPTNSILFFGRCISP |
| 67 | Ovalbumin-like | MGSIGPVSTEVCCDIFRELRSQSVQENVCYSPLLIISTLSMVYIGAKDNTKAQIEK |
| | [Corvus cornix | AIHFDKIPGFGESTESQCGTSVSIHTSLKDIFTQITKPSDNYSISIARRLYAEEKY |
| | cornix] | PILPEYIQCVKELYKGGLESISFQTAAEKSRELINSWVESQTNGTIKNILQPSSVS |
| | | SQTDMVLVSAIYFKGLWEKAFKEEDTQTIPFRITEQESKPVQMMSQIGTFKVAEIP |
| | | SEKCRILELPYASGRLSLWVLLPDDISGLEQLETAITFENLKEWTSSSKMEERKIR |
| | | VYLPRMKIEEKYNLTSVLKSLGITDLFSSSANLSGISSAESLKVSAAFHEASVEIY |
| | | EAGSKGVGSSEAGVDGTSVSEEIRADHPFLFLIKHNPSDSILFFGRCFSP |
| 68 | PREDICTED: | MGSIGAASTEFCFDVFKELKVQHVNENIIISPLSIISALSMVYLGAREDTRAQIDK |
| | Ovalbumin-like | VVHFDKITGFGEAIESQCPTSESVHASLKETFSQLTKPSDNYSLAFASRLYAEETY |
| | [Calypte anna] | PILPEYLQCVKELYKGGLETINFQTAAEQARQVINSWVESQTDGMIKSLLQPSSVD |
| | | PQTEMILVNAIYFRGLWERAFKDEDTQELPFRITEQESKPVQMMSQIGSFKVAVVA |
| | | SEKVKILELPYASGQLSMLVLLPDDVSGLEQLESSITVEKLIEWISSNTKEERNIK |
| | | VYLPRMKIEEKYNLTSVLVALGITDLFSSSANLSGISSAESLKISEAVHEAFVEIQ |
| | | EAGSEVVGSPGPEVEVTSVSEEWKADRPFLFLIKHNPTNSILFFGRYISP |
| 69 | PREDICTED: | MGSIGPVSTEVCCDIFRELRSQSVQENVCYSPLLIISTLSMVYIGAKDNTKAQIEK |
| | Ovalbumin | AIHFDKIPGFGESTESQCGTSVSIHTSLKDIFTQITKPSDNYSISIARRLYAEEKY |
| | [Corvus | PILQEYIQCVKELYKGGLESISFQTAAEKSRELINSWVESQTNGTIKNILQPSSVS |
| | brachyrhynchos] | SQTDMVLVSAIYFKGLWEKAFKEEDTQTIPFRITEQESKPVQMMSQIGTFKVAEIP |
| | | SEKCRILELPYASGRLSLWVLLPDDISGLEQLETSITFENLKEWTSSSKMEERKIR |
| | | VYLPRMKIEEKYNLTSVLKSLGITDLFSSSANLSGISSAESLKVSAVFHEASVEIY |
| | | EAGSKGVGSSEAGVDGTSVSEEIRADHPFLFLIKHNPSDSILFFGRCFSP |
| 70 | Hypothetical | MLNLMHPKQFCCTMGSIGPVSTEVCCDIFRELRSQSVQENVCYSPLLIISTLSMVY |
| | protein | IGAKDNTKAQIEKAIHFDKIPGFGESTESQCGTSVSIHTSLKDIFTQITKPSDNYS |
| | DUI87_08270 | ISIASRLYAEEKYPILPEYIQCVKELYKGGLESISFQTAAEKSRELINSWVESQTN |
| | [Hirundo rustica | GTIKNILQPSSVSSQTDMVLVSAIYFKGLWEKAFKEEDTQTVPFRITEQESKPVQM |
| | rustica] | MSQIGTFKVAEIPSEKCRILELPYASGRLSLWVLLPDDISGLEQLETAITSENLKE |
| | | WTSSSKMEERKIKVYLPRMKIEEKYNLTSVLKSLGITDLFSSSANLSGISSAESLK |
| | | VSGAFHEAFVEIYEAGSKAVGSSGAGVEDTSVSEEIRADHPFLFFIKHNPSDSILF |
| | | FGRCFSP |
| 71 | Ostrich OVA | EAEAGSIGTASAEFCFDVFKELKVHHVNENIFYSPLSIISALSMVYLGARENTKTQ |
| | sequence as | MEKVIHFDKITGLGESMESQCGTGVSIHTALKDMLSEITKPSDNYSLSLASRLYAE |
| | secreted from | QTYAILPEYLQCIKELYKESLETVSFQTAADQARELINSWIESQTNGVIKNFLQPG |
| | pichia | SVDSQTELVLVNAIYFKGMWEKAFKDEDTQEVPFRITEQESRPVQMMYQAGSFKVA |
| | | TVAAEKIKILELPYASGELSMLVLLPDDISGLEQLETTISFEKLTEWTSSNMMEDR |
| | | NMKVYLPRMKIEEKYNLTSVLIALGMTDLFSPAANLSGISAAESLKMSEAIHAAYV |
| | | EIYEADSEIVSSAGVQVEVTSDSEEFRVDHPFLFLIKHNPTNSVLFFGRCISP |
| 72 | Ostrich construct | MRFPSIFTAVLFAASSALAAPVNTTTEDETAQIPAEAVIGYSDLEGDEDVAVLPFS |
| | (secretion | NSTNNGLLFINTTIASIAAKEEGVSLEKREAEAGSIGTASAEFCFDVFKELKVHHV |
| | signal + mature | NENIFYSPLSIISALSMVYLGARENTKTQMEKVIHFDKITGLGESMESQCGTGVSI |
| | protein) | HTALKDMLSEITKPSDNYSLSLASRLYAEQTYAILPEYLQCIKELYKESLETVSFQ |
| | | TAADQARELINSWIESQTNGVIKNFLQPGSVDSQTELVLVNAIYFKGMWEKAFKDE |
| | | DTQEVPFRITEQESRPVQMMYQAGSFKVATVAAEKIKILELPYASGELSMLVLLPD |
| | | DISGLEQLETTISFEKLTEWTSSNMMEDRNMKVYLPRMKIEEKYNLTSVLIALGMT |
| | | DLFSPAANLSGISAAESLKMSEAIHAAYVEIYEADSEIVSSAGVQVEVTSDSEEFR |
| | | VDHPFLFLIKHNPTNSVLFFGRCISP |
| 73 | Duck OVA | EAEAGSIGAASTEFCFDVFRELRVQHVNENIFYSPFSIISALAMVYLGARDNTRTQ |
| | sequence as | IDKVVHFDKLPGFGESMEAQCGTSVSVHSSLRDILTQITKPSDNFSLSFASRLYAE |
| | secreted from | ETYAILPEYLQCVKELYKGGLESISFQTAADQARELINSWVESQINGIIKNILQPS |
| | pichia | SVDSQTTMVLVNAIYFKGMWEKAFKDEDTQAMPFRMTEQESKPVQMMYQVGSFKVA |
| | | MVTSEKMKILELPFASGMMSMFVLLPDEVSGLEQLESTISFEKLTEWTSSTMMEER |
| | | RMKVYLPRMKMEEKYNLTSVFMALGMTDLFSSSANMSGISSTVSLKMSEAVHAACV |
| | | EIFEAGRDVVGSAEAGMDVTSVSEEFRADHPFLFFIKHNPTNSILFFGRWMSP |
| 74 | Duck construct | MRFPSIFTAVLFAASSALAAPVNTTTEDETAQIPAEAVIGYSDLEGDFDVAVLPFS |
| | (secretion | NSTNNGLLFINTTIASIAAKEEGVSLEKREAEAGSIGAASTEFCFDVFRELRVQHV |
| | signal + mature | NENIFYSPFSIISALAMVYLGARDNTRTQIDKVVHFDKLPGFGESMEAQCGTSVSV |
| | protein) | HSSLRDILTQITKPSDNFSLSFASRLYAEETYAILPEYLQCVKELYKGGLESISFQ |
| | | TAADQARELINSWVESQTNGIIKNILQPSSVDSQTTMVLVNAIYFKGMWEKAFKDE |
| | | DTQAMPFRMTEQESKPVQMMYQVGSFKVAMVTSEKMKILELPFASGMMSMFVLLPD |
| | | EVSGLEQLESTISFEKLTEWTSSTMMEERRMKVYLPRMKMEEKYNLTSVFMALGMT |
| | | DLFSSSANMSGISSTVSLKMSEAVHAACVEIFEAGRDVVGSAEAGMDVTSVSEEFR |
| | | ADHPFLFFIKHNPTNSILFFGRWMSP |

### EXAMPLES

### Example 1: Methods for Recovering and Purifying an Illustrative Protein of Interest

FIG. 1A is a flow chart that illustrates illustrative steps of methods of the present disclosure.

The steps of the illustrated method include: obtaining recombinant fungal cells capable of expressing a secreted protein of interest; culturing the recombinant fungal cells under conditions that promote expression and secretion of the recombinant protein of interest into a culturing medium; centrifuging the culturing medium and excluding the recombinant fungal cells and other cellular components; microfiltering the centrifuged culturing medium to further remove any residual cell components prior to adding the acid and introducing ammonium sulfate; adding an acid to the microfiltered culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; recovering the precipitated protein of interest; solubilizing the precipitated secreted protein of interest with water to obtain a solubilized protein of interest; diafiltering and/or ultrafiltering the solubilized protein of interest; microfiltering the diafiltered and/or ultrafiltered protein of interested; and drying the further microfiltered protein of interest, thereby obtaining a dried protein product.

In some cases, one more of the above steps is omitted. For example, the method may conclude when the protein of interest is precipitated and recovered. In this case, the method comprises steps of: obtaining recombinant fungal cells capable of expressing a secreted protein of interest; culturing the recombinant fungal cells under conditions that promote expression and secretion of the recombinant protein of interest into a culturing medium; adding an acid to the culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; and recovering the precipitated protein of interest.

Alternately, the method comprises steps of: obtaining recombinant fungal cells capable of expressing a secreted protein of interest; culturing the recombinant fungal cells under conditions that promote expression and secretion of the recombinant protein of interest into a culturing medium; centrifuging the culturing medium and excluding the recombinant fungal cells and other cellular components; microfiltering the centrifuged culturing medium to further remove any residual cell components prior to adding the acid and introducing ammonium sulfate; adding an acid to the microfiltered culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; and recovering the precipitated protein of interest.

### Example 2: Analysis of pH Effects on Recovery of Protein of Interest

In a first series of experiments, the effect of pH in recovery and purity of a protein of interest was assayed. Here, the illustrative protein was recombinant ovalbumin (rOVA). The pH values, which were selected to be about the isoelectric point (pI) of OVA, included 3.5, 4, 4.25, 4.5, 4.75, 5, 5.25, and 5.5.

In these experiments, two vials (30-35 mLs each) of frozen culturing medium containing secreted protein of interest were obtained. Before freezing, fungal cells (Aspergillus Niger) that expressed rOVA were cultured in the culturing medium into which the rOVA was secreted. Also, before freezing, the culturing medium was centrifuged and microfiltered to remove any residual cell components. The frozen culturing medium was thawed to room temperature. The vials were diluted 2-fold. Samples of the secreted protein of interest was collected before and after dilution. Using external material post-MFI split into 16 samples of about 5 mL each and were placed into 15 mL conical centrifuge tube. Control samples were also collected. For each pH assessment there were two duplicate samples. The temperature and agitation were identical for all conditions to reduce the variation in the analysis. Experiments were done at room temperature, and all conditions were stirred using the tube roller. To each tube, 85% phosphoric acid (v/v) was added to reach the desired pH. The amount of added acid was measured. The tubes were mixed well and placed on a tube roller or gel waver for an hour. Precipitation was noted. The tubes were centrifuged at 6500 RCF for about 30 min. The supernatant was decanted and the vials.

Data is shown in the below Table 2.

TABLE 2

| | | | | | | Total solids |
|---|---|---|---|---|---|---|
| | | Temperature | pH | Appearance | Acid added | % |
| | Pre-dilution | 20.7 C. | 5.69 | Brown, like | | |
| | | | | iodine | | |
| | Post-Dilution | 20.6 | 5.75 | Brown, | | |
| | | | | lighter in | | |
| | | | | color | | |
| 1 | pH 5.5 | 20.2 | 5.48 | Brown, clear | 0.3 mL 100× | 0.00% |
| | | | | Very small | | (resuspended |
| | | | | pellet | | in 1 mL) |
| 2 | pH 5.5 | 20.3 | 5.50 | Brown, clear | 0.28 mL 100× | 0.00% |
| | | | | Very small | | 0.944 g total |
| | | | | pellet | | |
| 3 | pH 5.25 | 19.9 | 5.26 | Brown, clear | .7 mL 100× | 0.931 g total |
| | | | | Very small | | 0% |
| | | | | pellet | | |
| 4 | pH 5.25 | 19.8 | 5.25 | Brown, clear | 0.7 ml 100× | 0% |
| | | | | Very small | | |
| | | | | pellet | | |
| 5 | pH 5 | 20.1 | 5.03 | Brown, clear | 0.1 mL 10× | 0% |
| | | | | Small pellet | | |
| 6 | pH 5 | 20.1 | 5.05 | Brown, clear | 0.1 mL 10× | 0% |
| | | | | Small pellet | | |
| 7 | pH 4.75 | 19.7 | 4.76 | Slight haze | .11 mL 10× | 0% |
| | | | | Pellet smaller | 4 mL 100× | |
| | | | | than 3.5 | | |
| 8 | pH 4.75 | 19.9 | 4.76 | Pellet smaller | | 0 |
| | | | | than 3.5 | | |
| 9 | pH 4.5 | 20.2 | 4.52 | hazy | | 0% |
| 10 | pH 4.5 | 19.9 | 4.53 | hazy | 1.3 mL 10× | .17% (1.77 g |
| | | | | | + | to 0.003 g) |
| 11 | pH 4.25 | 19.9 | 4.27 | hazy | 1.5 mL 10× | 0.4% (0.995 g |
| | | | | | .3 mL 100× | to 0.04 g) |
| 12 | pH 4.25 | 20.0 | 4.22 | hazy | 1.7 mL 10× | 0.51% |
| | | | | | | (0.982->0.05) |
| 13 | pH 4 | | 3.98 | hazy | | 0.91% |
| | | | | | | (0.984->0.009) |
| 14 | pH 4 | | 4.02 | Hazy | .2 mL 10× | 0.036% (in |
| | | | | Large Pellet | .4 mL 100× | 2 mLs)(1.929- |
| | | | | | | >0.007 g) |
| 15 | pH 3.5 | 19.5 | 3.54 | Yellow, hazy | .5 mLs 10× | 1.93 g initially |
| | | | | Pellet settled | | (pellet and |
| | | | | after some | | 2 mL water) |
| | | | | time | | 0.019 g final |
| | | | | Large pellet | | 0.083% |
| 16 | pH 3.5 | 19.0 | 3.52 | Yellow, hazy, | 3 mLs 100×, | 2.12% (0.949 g |
| | | | | turned hazy | .3 mLs 10× | initially, |
| | | | | around 3.8, | | 0.021 g final |
| | | | | Pellet settled | | |
| | | | | after some | | |
| | | | | time | | |
| | | | | Large Pellet | | |
| 17 | pH 3.75 | | 3.79 | Yellow, hazy | .4 mL 10× | 1.38% DC |
| | | | | Large Pellet | | (resuspended in |
| | | | | | | 1 mL) (0.014 g |
| | | | | | | of 1.) |
| 18 | pH 3.75 | | 3.75 | Large Pellet | .4 mL 10× | 0.26% |
| | | | | | | (resuspended in |
| | | | | | | 5 mL) 4.925 g |
| | | | | | | total, 0.013 g |
| | | | | | | final |
| 19 | pH 3.25 | | 3.21 | Large Pellet | .5 mL 10× | 1.39% in |
| | | | | | | 2 mL (2.015 g |
| | | | | | | to 0.028 g) |
| 20 | pH 3.25 | | 3.26 | Large Pellet | .5 mL 10× | 1.27% (1.974 |
| | | | | | | to 0.025 g) |

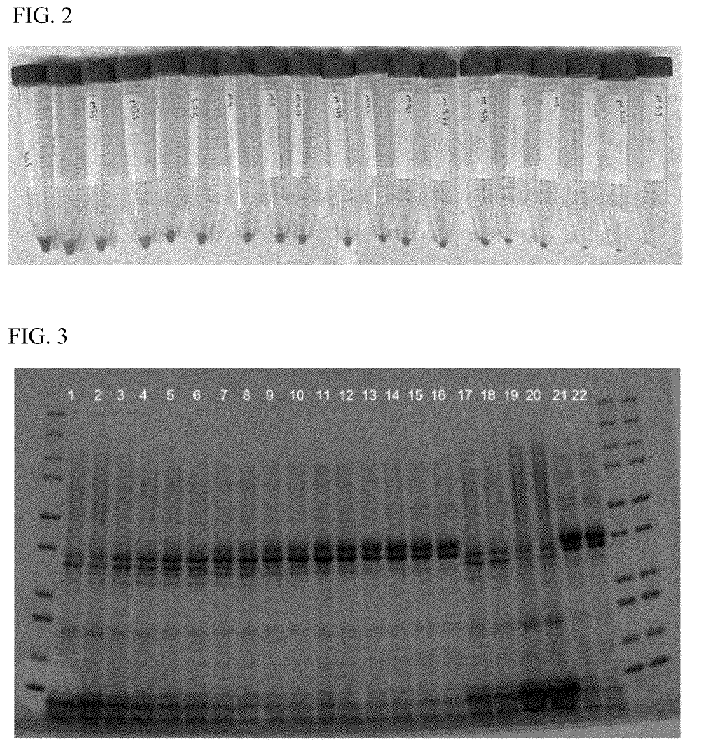

FIG. 2 is a photograph showing centrifuge tubes containing pelleted protein of interest. The proteins were precipitated with increasing pH from left to right (pH 3.25 to pH 5.5).

Later, the pellets were again centrifuged, and the supernatant was decanted and moisture analysis was conducted. Then, 1 to 2 ml of water was added to each conical tube and the pellets were vortexed to suspend the protein of interest.

FIG. 3 is a SDS PAGE gel for proteins precipitated at various pH values.

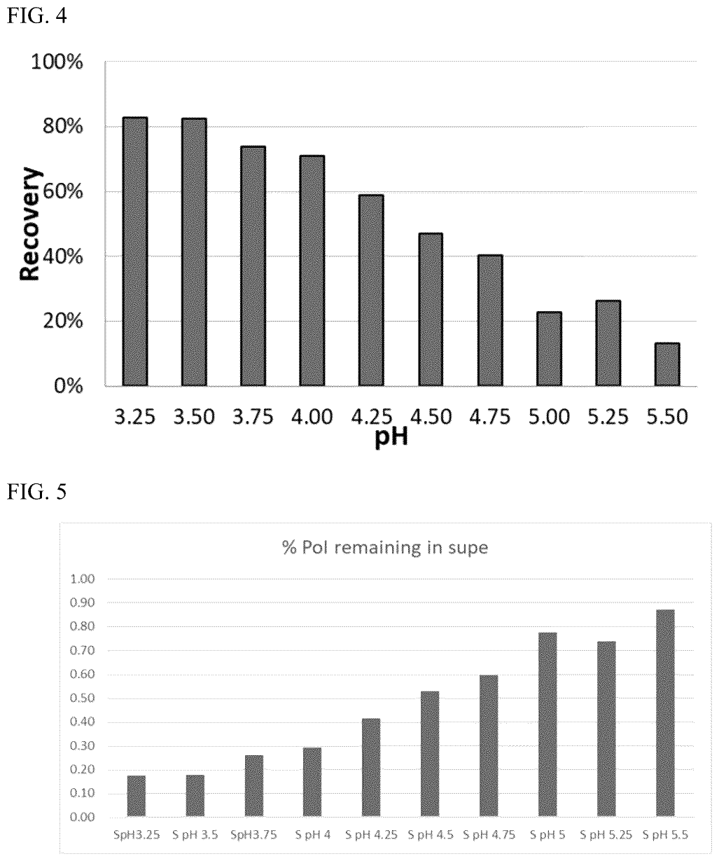

Additional data is shown in the following table (Table 3), which is characterized in FIG. 4 and FIG. 5.

TABLE 3

| Sample | pH | Total Protein | POI amount |
|---|---|---|---|
| 1 | 3.50 | 2.02 | 1.888 |
| 2 | 3.50 | 2.26 | 2.273 |
| 3 | 4.00 | 3.46 | 3.292 |
| 4 | 4.00 | 3.62 | 3.594 |
| 5 | 4.25 | 4.63 | 4.643 |
| 6 | 4.25 | 4.92 | 5.166 |
| 7 | 4.50 | 6.87 | 6.266 |
| 8 | 4.50 | 7.13 | 6.295 |
| 9 | 4.75 | 9.26 | 7.21 |
| 10 | 4.75 | 8.7 | 6.947 |
| 11 | 5.00 | 10.36 | 9.059 |
| 12 | 5.00 | 10.48 | 9.288 |
| 13 | 5.25 | 9.99 | 8.748 |
| 14 | 5.25 | 9.48 | 8.731 |
| 15 | 5.50 | 9.92 | 10.125 |
| 16 | 5.50 | 10.66 | 10.489 |
| 17 | 3.75 | 3.3 | 3.191 |
| 18 | 3.75 | 3.31 | 3.008 |
| 19 | 3.25 | 1.25 | 2.039 |
| 20 | 3.25 | 1.18 | 2.081 |
| 21 | Pre- | 18.381 | 24.068 |
| | Dilution | | |
| 22 | Post- | 11.673 | 11.857 |
| | Dilution | | |

FIG. 4 is a graph showing percentages of protein recovery for proteins precipitated at various pH values. As shown, at pH 3.5 and pH 3.25 the maximum protein recovery was observed, i.e., over 80%.

FIG. 5 is a graph showing percentages of protein that was not recovered, i.e., proteins remaining in the supernatant, for proteins precipitated at various pH values. The percentages shown on the Y-axis are in decimal; thus, “0.1” means “10%”. POI: protein of interest; supe: supernatant. As shown, at pH 3.5 and pH 3.25 the least amount of protein lost in the supernatant was observed, i.e., about 17%.

As shown in FIG. 4 and FIG. 5 and Table 3 a Total Solids analysis was conducted with the pellets. The data shows that as pH decreased, protein precipitation increased. pH 3.25 and pH 3.5 performed very similarly (17.5 and 17.3% of protein of interest remaining in the supernatant, respectively). These pH levels showed the best results out of all conditions, i.e., the least protein remaining in supernatant after centrifugation. The visual pellet sizes (shown in FIG. 2) further demonstrate the superiority of the lower pH. Apparently, the pI of the rOVA used in the example appeared to be between 3.25 and 3.5, and precipitation was best achieved at these pH levels. Interestingly the pI of the rOVA used in this Example was about 3.5 instead of 4.1, which is the pI for natural OVA.

Notably, as shown in FIG. 3, the gel of the low pH samples shows very high impurity. This suggests that precipitating by pH reduction alone does not provide desirable recovery of highly pure protein of interest. Thus, use of salt in conjunction with the reduced pH may be warranted.

### Example 3: Analysis of Salt Effects on Recovery and Purity of Protein of Interest

In a second series of experiments, the effect of salt (ammonium sulfate) in recovery and purity of a protein of interest was assayed. Here, the illustrative protein was recombinant ovalbumin (rOVA). In these experiments, the pH of the culturing medium containing secreted protein of interest was lowered (to pH 4 or pH 4.5) before adding ammonium sulfate. The salt concentrations tested were 100 g/l, 200 g/l, 300 g/l, and 400 g/l. The precipitation steps performed were similar to those of Example 2.

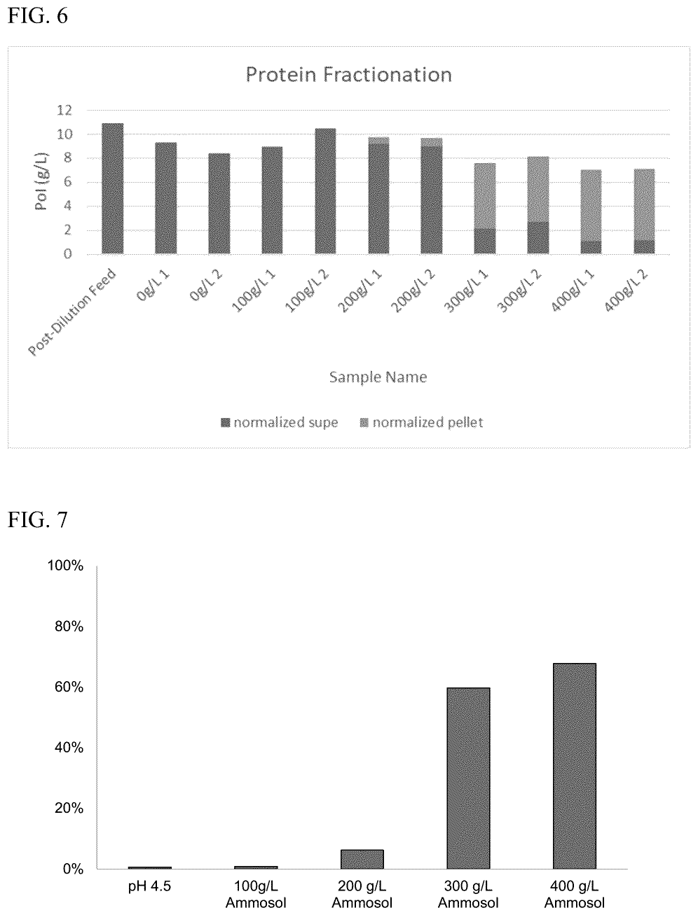

FIG. 6 is a graph showing the amount of protein (in grams/liter) recovered (in blue and bottom portion of each bar) and the amount of protein that was not recovered, i.e., proteins remaining in the supernatant (in orange and top portion of each bar), for proteins precipitated at various concentrations of ammonium sulfate. As shown, a salt concentration of 300 g/l or 400 g/l gave superior recovery of precipitated protein and minimized loss of protein in the supernatant.

FIG. 7 is a graph showing the percentage of protein recovered for proteins precipitated at various concentrations of ammonium sulfate, with pH at 4.5 throughout. Similar to FIG. 6, 300 g/l and 400 g/l showed superior recovery of precipitated protein.

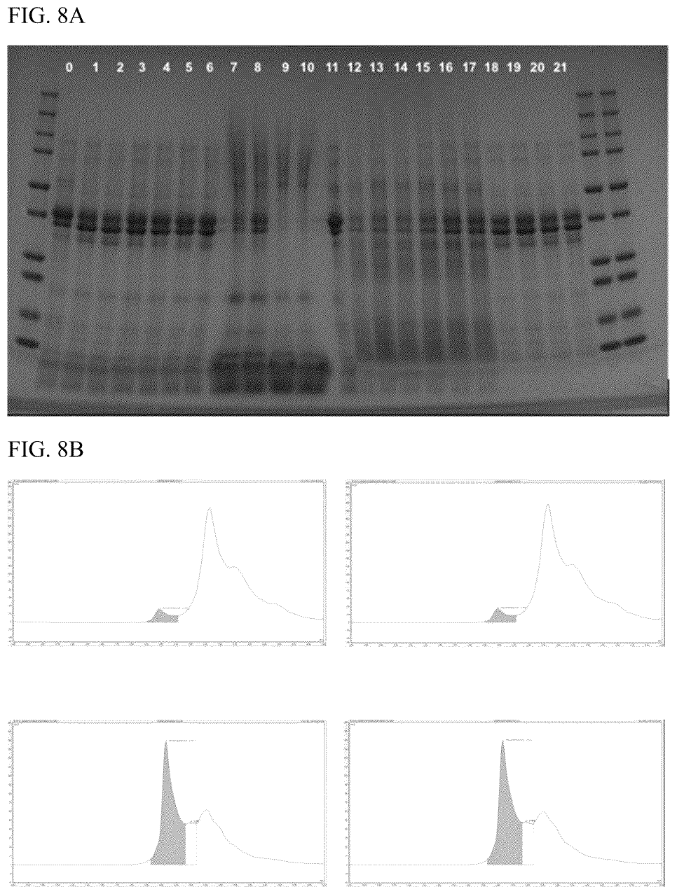

FIG. 8A is an SDS PAGE gel demonstrating the purification of the protein with certain process conditions; lanes 9 and 10 are duplicates of the supernatant and lanes 20 and 21 are the respective pellets resuspended in DI water to the initial volume. Comparing the lanes 7,8 at pH 4.5 of FIG. 3 to lanes 20,21 of FIG. 8A where the pH 4.5 solution was then further modified with addition of 400 g/L equivalent of ammonium sulfate shows that salt precipitation leads to more pure samples.

FIG. 8B includes chromatograms (top two images) for supernatant samples 9 and 10 of FIG. 8A and chromatogram (bottom two images) for precipitant samples 20 and 21. The chromatograms show that combined reduced pH with high salt precipitation provides good recovery of relative pure protein of interest.

Together, the combination of a pH about or below the pI of a protein of interest (for rOVA, a pH of less than 4.5, preferably in the pH 3 range) and a high salt concentration (e.g., 300 g/l or 400 g/l ammonium sulfate) provides superior recovery of pure protein of interest.

### Example 4: Methods Comprising Additional Steps for Recovery and Purity of Protein of Interest

In this example, methods for recovering pure protein of interest were assayed.

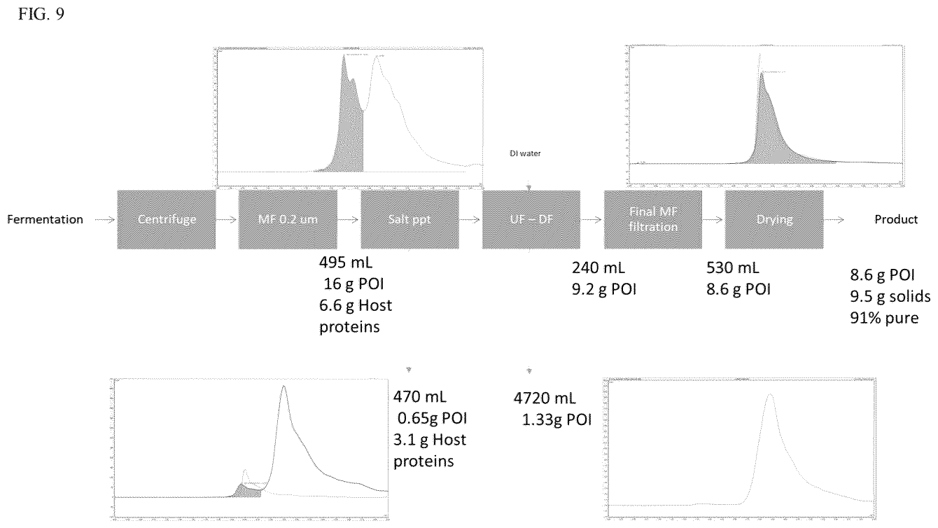

FIG. 9 includes a flow chart of a first run of a recovery method of the present disclosure. Shown are chromatograms showing recovery and purity of protein at various stages in the method and also shown are amounts of protein of interest (POI) recovered at the various stages. MF: microfiltration; UF-DF: ultrafiltration-diafiltration. As shown, in the top right chromatogram, the ultimate protein product was highly pure (i.e., 91% pure protein of interest) and the method recovered over 53% of the original protein of interest.

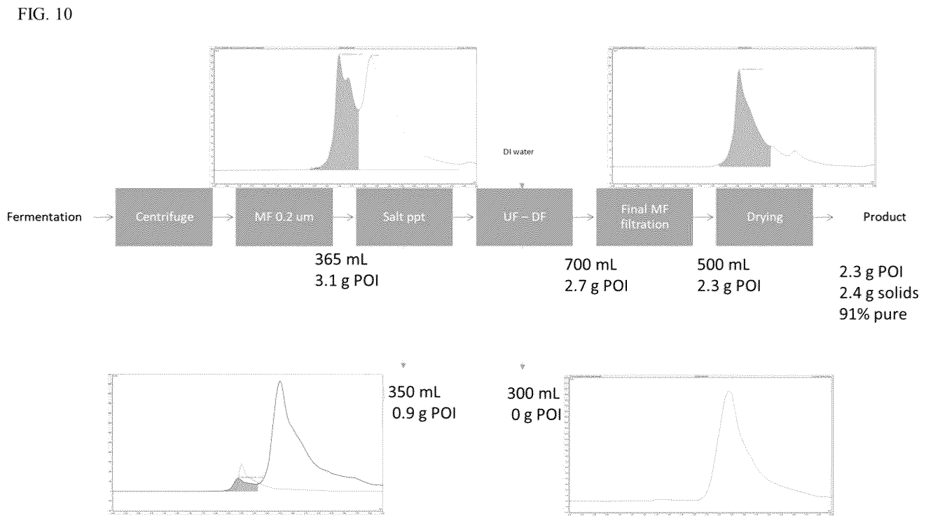

FIG. 10 includes a flow chart of a second run of a recovery method of the present disclosure. Shown are chromatograms showing recovery and purity of protein at various stages in the method and also shown are amounts of protein of interest (POI) recovered at the various stages. MF: microfiltration; UF-DF: ultrafiltration-diafiltration. As shown, in the top right chromatogram, the ultimate protein product was highly pure (i.e., 91% pure protein of interest) and the method recovered over 74% of the original protein of interest.

### Example 5: Illustrative Process Scale Up

This example describes a downstream processing of recombinantly-expressed proteins under current food good manufacturing processes (cGMP).

A fungal platform, as disclosed herein, is used to produce an egg white protein through a fermentation process. The protein of interest is secreted extracellularly. The secreted protein of interest undergoes downstream processing as depicted in FIG. 1A and the overall process is as follows: Once fermentation is complete, the fermentation broth is diluted, chilled, and clarified using a centrifuge. The solids are disposed of and the centrate is filtered through a 0.2 μm filter in a TFF mode (hollow fiber/spiral wound) to remove any remaining cell debris. The filtrate may be stored at 8° C. for up to 72 hours at this point. Following clarification, the permeate from the 0.2 μm filtration is reduced in volume by a 10 kDa membrane concentrating by about six to about eight-fold. Ammonium sulfate is then added to the retentate to precipitate the protein of interest. This precipitate is recovered using a centrifuge or Sedicanter®. The precipitate is resuspended to a protein concentration target of 40-50 g/L and the pH adjusted to about 6. This resuspension is dialyzed using a 10 kDa membrane to reduce the conductivity to below 900 μS/cm. Prior to drying the protein product into a powder, 0.2 μm filtration is performed as a step to reduce bioburden and then the final product is spray dried. All final product packaging material is suitable for food grade use.

### Detailed Process Description

If material is held for more than four hours without processing, it is kept chilled between 8 and 15° C. to minimize microbial growth.

In the “Precipitation” steps, solid separation occurs via centrifugation; clarification occurs via 0.2 μm Filtration UF+DF, and Precipitation in ammonium sulfate (e.g., 65% w/v).

Precipitation Parameters

| | |
|---|---|
| Ammonium Sulfate target | 40% w/v |
| concentration | |
| Temperature | 20° C.-25° C. |
| pH | pH 4.5 |
| Precipitation Time | 4-12 hours |
| Mixing Conditions | Gentle stirring |

The purpose of the ammonium sulfate precipitation step is to further purify the protein. First, ammonium sulfate is added to the retentate from the 10 kDa Ultrafiltration step to create a 40% w/v ammonium sulfate solution. Following salt addition, the pH is adjusted to 4.5 with 85% orthophosphoric acid.

Proper temperature, pH, salt concentration, and mixing control appear to provide proper formation and growth of the precipitate. Moderate agitation and tank chilling (as compared to using an external heat exchanger and pump) appear to be helpful. Slow addition of the salt with moderate mixing helps allow the salt to fully dissolve and avoids clumps and poor precipitation. When mixed properly, a milky white precipitate will form, whereas hard agitation will cause foaming.

Precipitation continues for about four hours to about twelve depending on mixing and rate of precipitation. Tracking the progress of precipitation with bench centrifugation spin test can demonstrate when the precipitation has stalled out. Further salt or acid additions may be needed to restart precipitation. Monitoring should continue until no further change can be affected or are detected.

The precipitate recovery and dilution are performed according to the following:

Precipitation Recovery and Resuspension Parameters

| Final Target Protein Concentration | 40-50 g/L |
|---|---|
| Final pH | 6 ± 0.1 |

The precipitate is recovered by use of a centrifuge or a Sedicanter®. Use of a disk stack centrifuge can be challenging due to the physical nature of the protein precipitate. A balance of the feed solids percentage, the feed rate, and the residence time in the bowl helps prevent the solids from adhering to the bowl. In the event solids do adhere, a water rinse through the machine and cyclone is performed and which is not allowed to go out the centrate. This rinse can recover the protein as the POI is very soluble. Typical disk stack centrifuges have wash nozzles on the bowl and cyclone to help remove solids. Notably, the Sedicanter® from Flottweg can provide more efficient recovery of precipitate than a standard centrifuge.

The resultant solid slurry is resuspended back up to the desired volume to ensure complete solubilization. A protein concentration target of about 40 to about 50 g/L will assist proper solubilization; the pH should be increased to about 6 or about 6.5 with sodium hydroxide. This process can continue until no precipitate is seen in suspension.

The suspension is filtered/dialyzed using a 10 kDa Diafiltration and according to the following:

10 kDa parameters

| | Polyether sulfone (PES) hollow |
|---|---|
| Membrane Type | fiber/spiral wound (recommended) |
| Membrane size | 10 kDa nominal |
| Temperature | 10° C. ± 5 |
| Final pH | 6.9 ± 0.2 |
| Transmembrane | Up to 2 bar (follow manufacturer |
| pressure (TMP) | recommendation) |
| Concentration | At 50 g/L |
| Average Flux | 5 ± 2 LMH |
| Retentate Conductivity | <1 mS/cm |
| Color of retentate | Clear, golden yellow |

The purpose of the 10 kDa diafiltration is to remove salts from the solution. This filtration is run in a tangential mode. The target protein is in the retentate. The solution at pH 6.5±0.2 at ˜50 g/L protein concentration is diafiltered until the conductivity in the retentate is <900 uS/cm. The final dialyzed material should be golden yellow and clear and at about pH 6.9±0.2. Typically, this would mean diafiltering around 6-8 DVs.

The final dialyzed material undergoes 0.2 μm filtration and spray drying. The final step in the downstream process involves polishing wherein the final product is obtained by spray drying. However, prior to spray drying, the ultrafiltered retentate can be sterile filtered using a 0.2 μm MF filter. This needs to be done to reach our target specifications on the microload of our product. Parameters are as follows:

0.2 um Tangential Flow Filtration parameters

| | Polyether sulfone (PES) |
|---|---|
| Membrane type | hollow fiber/spiral wound |
| Membrane size | 0.2 μm nominal |
| Temperature | 10° C. ± 5 |
| Final pH | 6.9 ± 0.2 |
| Transmembrane pressure (TMP) | Up to 2 bar (follow manufacturer |
| | recommendation) |
| Average flux (expected) | 10 ± 2 LMH |
| Color of final product | Clear yellow/gold |

The filtered material is then spray dried at the following conditions: Inlet temperature: 165° C., Outlet temperature: 65-67° C., and Air inlet: 3 bar

The final powdered product shall meet the following specifications depending on the type of product:
Moisture content 5 to 10% w/w
Protein content>80% by bradford assay
Micro levels
   Standard Plate Count less than 5000 CFU/g
   Yeast and Mold less than 10 CFU/g
   Salmonella not detected in 25 g
   E-coli not detected in 25 g
Heavy metals levels
   Mercury<0.1 ppm
   Arsenic<0.2 ppm
   Lead<0.5 ppm
   Cadmium<0.1 ppm

### Example 6: Properties of an Illustrative Recovered Protein of Interest

In this example, properties of a recovered protein of interest were assayed. Here, the recovered protein of FIG. 9 was evaluated for functionality.

The recovered rOVA provided a foam Capacity that was higher than a control OVA sample.

The recovered rOVA provided a foam stability that was slightly higher than a control OVA sample

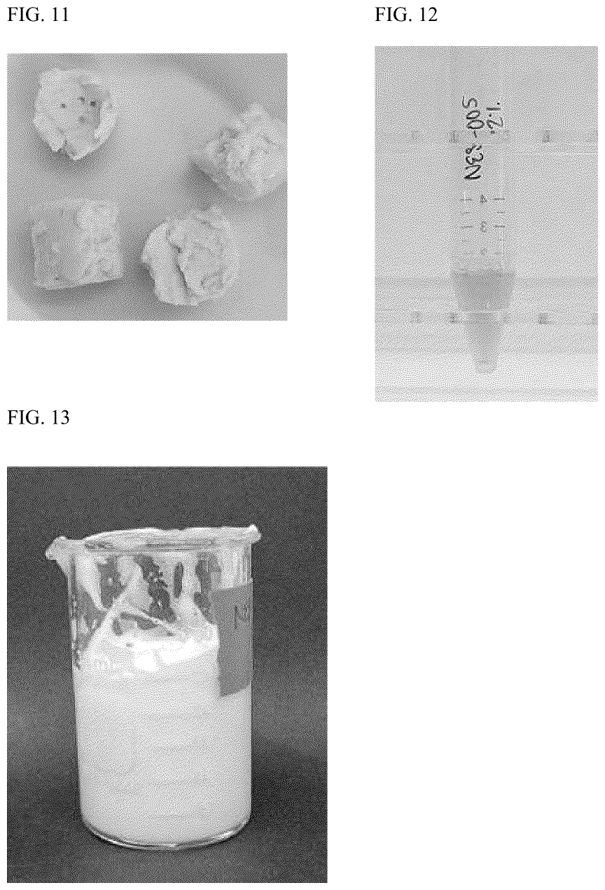

The recovered rOVA provided a hardness that was slightly improved from the control OVA sample; however, gumminess and chewiness were significantly increased. See, FIG. 11.

The recovered rOVA provided absorbance of 0.109 showing the clarity of solubilized rOVA.

When in a 12% (w/w) solution in water, the recovered rOVA had a pH of 5.85, a conductivity of 2.50 ms. See, FIG. 12.

Notably, the 12% (w/w) recovered rOVA solution had foam capacity that was 650%. See, FIG. 13. It had a foam stability that was 65%.

Further data is shown below in Table 4.

TABLE 4

| | |
|---|---|
| Hardness | 449.20 ± 103.85 |
| Adhesiveness | 0.82 ± 0.66 |
| Hardness | 404.87 ± 100.64 |
| Cohesiveness | 0.58 ± 0.06 |
| Springiness | 4.60 ± 0.26 |
| Gumminess | 258.93 ± 59.76 |
| Chewiness | 11.77 ± 3.28 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for increasing recovery and purity of a secreted protein of interest, the method comprising steps of:
obtaining recombinant fungal cells capable of expressing a secreted protein of interest;
culturing the recombinant fungal cells under conditions that promote expression and secretion of the recombinant protein of interest into a culturing medium;
adding an acid to the culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; and
recovering the precipitated protein of interest.

2. The method of claim 1, wherein the method comprises a step of centrifuging the culturing medium and excluding the recombinant fungal cells and other cellular components after culturing the recombinant fungal cells.

3. The method of any one of the preceding claims, wherein the method comprises a step of microfiltering the centrifuged culturing medium to further remove any residual cell components prior to adding the acid and introducing ammonium sulfate.

4. The method of any one of the preceding claims, wherein the microfiltering comprises a filter capable of capturing fungal cells and other cellular components.

5. The method of claim 4, wherein the filter is a 0.2 μm filter.

6. The method of any one of the preceding claims, wherein the method comprises a step solubilizing the precipitated secreted protein of interest with water, e.g., DI water, to obtain a solubilized protein of interest.

7. The method of any one of the preceding claims, wherein the method comprises a step diafiltering and/or ultrafiltering the solubilized protein of interest.

8. The method of any one of the preceding claims, wherein the method comprises a step of further microfiltering the diafiltered and/or ultrafiltered protein of interested.

9. The method of any one of the preceding claims, wherein the method comprises a step of drying the further microfiltered protein of interest, thereby obtaining a dried protein product.

10. The method of any one of the preceding claims, wherein the pH is reduced to a pH below 5, below 4.75, below 4.5, below 4.25, below 4.0, below 3.75, below 3.5, or below 3.25.

11. The method of any one of the preceding claims, wherein the pH is reduced to a pH of about 5, about 4.75, about 4.5, about 4.25, about 4.0, about 3.75, about 3.5, or about 3.25.

12. The method of any one of the preceding claims, wherein the acid is phosphoric acid, e.g., 85% v/v phosphoric acid.

13. The method of any one of the preceding claims, wherein adding the acid occurs before introducing the ammonium sulfate.

14. The method of any one of the preceding claims, wherein adding the acid occurs after introducing the ammonium sulfate.

15. The method of any one of the preceding claims, wherein adding the acid is contemporaneous with introducing the ammonium sulfate.

16. The method of any one of the preceding claims, wherein the ammonium sulfate concentration is above 200 g/l, the ammonium sulfate concentration is above 300 g/l, or the ammonium sulfate concentration is above 400 g/l.

17. The method of any one of the preceding claims, wherein the ammonium sulfate concentration is about 200 g/l, the ammonium sulfate concentration is about 300 g/l, or the ammonium sulfate concentration at is about 400 g/l.

18. The method of any one of the preceding claims, wherein the pH is about or below 4.75, is about or below 4.5, is about or below 4.25, is about or below 4.0, is about or below 3.75, is about or below 3.5, or is about or below 3.25 and wherein the ammonium sulfate concentration is about or above 200 g/l, the ammonium sulfate concentration is about or above 300 g/l, or the ammonium sulfate concentration about is or above 400 g/l.

19. The method of any one of the preceding claims, wherein the pH is about or below 4.75 and the ammonium sulfate concentration is about or above 300 g/l.

20. The method of any one of the preceding claims, wherein the pH is about or below 4.75 and the ammonium sulfate concentration is about or above 400 g/l.

21. The method of any one of the preceding claims, wherein the pH is about or below 4.5 and the ammonium sulfate concentration is about or above 300 g/l.

22. The method of any one of the preceding claims, wherein the pH is about or below 4.5 and the ammonium sulfate concentration is about or above 400 g/l.

23. The method of any one of the preceding claims, wherein the pH is about or below 4.25 and the ammonium sulfate concentration is about or above 300 g/l.

24. The method of any one of the preceding claims, wherein the pH is about or below 4.25 and the ammonium sulfate concentration is about or above 400 g/l.

25. The method of any one of the preceding claims, wherein the pH is about or below 4.0 and the ammonium sulfate concentration is about or above 300 g/l.

26. The method of any one of the preceding claims, wherein the pH is about or below 4.0 and the ammonium sulfate concentration is about or above 400 g/l.

27. The method of any one of the preceding claims, wherein the pH is about or below 3.75 and the ammonium sulfate concentration is about or above 300 g/l.

28. The method of any one of the preceding claims, wherein the pH is about or below 3.75 and the ammonium sulfate concentration is about or above 400 g/l.

29. The method of any one of the preceding claims, wherein the pH is about or below 3.5 and the ammonium sulfate concentration is about or above 300 g/l.

30. The method of any one of the preceding claims, wherein the pH is about or below 3.5 and the ammonium sulfate concentration is about or above 400 g/l.

31. The method of any one of the preceding claims, wherein the pH is about or below 3.25 and the ammonium sulfate concentration is about or above 300 g/l.

32. The method of any one of the preceding claims, wherein the pH is about or below 3.25 and the ammonium sulfate concentration is about or above 400 g/l.

33. The method of any one of the preceding claims, wherein the amount of recovered protein of interest is greater than the recovery that is obtained from a method that does not comprise reducing the pH of the culturing medium to about or below the pI of the protein of interest and does not comprise adding ammonium sulfate to the culturing medium to achieve an ammonium sulfate concentration above 200 g/l.

34. The method of any one of the preceding claims, wherein recovery of the protein of interest is at least about 40% w/w, is at least about 45% w/w, is at least about 50% w/w, is at least about 55% w/w, or is at least about 60% w/w for the final dried product, wherein the recovery is the weight of the protein of interest recovered in the final product relative to the weight of the protein of interest prior to adding the acid and introducing ammonium sulfate.

35. The method of any one of the preceding claims, wherein recovery of the protein of interest is at least about 40% w/w, is at least about 45% w/w, is at least about 50% w/w, is at least about 55% w/w, is at least about 60% w/w, is at least about 65% w/w, is at least about 70% w/w, is at least about 75% w/w, is at least about 80% w/w, is at least about 85% w/w, is at least about 90% w/w, for the final dried product, wherein the recovery is the weight of the protein of interest recovered in the final product relative to the sum of weight of the protein of recovered and the weight of the protein remaining in the supernatant following adding the acid and introducing ammonium sulfate.

36. The method of any one of the preceding claims, wherein the purity of recovered protein of interest is greater than the purity that is obtained from a method that does not comprise reducing the pH of the culturing medium to about or below the pI of the protein of interest and does not comprise adding ammonium sulfate to the culturing medium to achieve an ammonium sulfate concentration above 200 g/l.

37. The method of any one of the preceding claims, wherein the purity is at least about 75% w/w, is at least about 80% w/w, is at least about 85% w/w is at least about 90% w/w, is at least about 95% w/w for the final dried product, wherein the purity is defined as the weight of the protein of interest relative to the total weight of solid product.

38. The method of any one of the preceding claims, wherein the method does not comprise use of a purification resin and/or a purification column.

39. The method of any one of the preceding claims, wherein the fungal cells are of the species selected from Agaricus bisporus; Agaricus spp.; Aspergillus awamori; Aspergillus fumigatus; Aspergillus nidulans; Aspergillus niger; Aspergillus oryzae; Aspergillus oryzae; Aspergillus spp.; Colletotrichum gloeosporiodes; Colletotrichum spp.; Endothia parasitica; Endothia spp.; Fusarium graminearum; Fusarium solani; Fusarium spp.; Komagatella pastoris.; Komagatella phaffi; Mucor miehei; Mucor pusillus; Mucor spp.; Myceliophthora spp.; Myceliophthora thermophila; Neurospora crassa; Neurospora spp.; Penicillium (Talaromyces) emersonii; Penicillium camemberti; Penicillium canescens; Penicillium chrysogenum; Penicillium funiculosum; Penicillium purpurogenum; Penicillium roqueforti; Penicillium spp.; Pichia angusta; Pichia pastoris; Pichia pastoris; Pichia Pastoris “MutS” strain (Graz University of Technology (CBS7435MutS) or Biogrammatics (BG11)); Pichia spp.; Pleurotus ostreatus; Pleurotus spp.; Rhizomucor miehei; Rhizomucor pusillus; Rhizomucor spp.; Rhizopus arrhizus; Rhizopus oligosporus; Rhizopus oryzae; Rhizopus spp.; Trichoderma altroviride; Trichoderma reesei; Trichoderma spp.; Trichoderma vireus; Yarrowia lipolytica; and Yarrowia spp.

40. The method of claim 39, wherein the fungal cells are Aspergillus cells.

41. The method of claim 40, wherein the Aspergillus cells are of the species Aspergillus spp., Aspergillus awamori, Aspergillus fumigatus, Aspergillus nidulans, Aspergillus niger, or Aspergillus oryzae.

42. The method of any one of the preceding claims, wherein the protein of interest is a food protein.

43. The method of claim 42, wherein the food protein is used as nutritional, dietary, digestive, supplements, such as in food products and feed products.

44. The method of claim 42 or claim 43, wherein the food protein is a plant protein.

45. The method of any one of claims 42 to 44, wherein the food protein is an animal protein.

46. The method of claim 45, wherein the animal protein is an egg white protein.

47. The method of claim 46, wherein the egg white protein is selected from ovalbumin, ovomucoid, ovotransferrin, lysozyme, ovomucin, ovoglobulin G2, ovoglobulin G3, ovoinhibitor, ovoglycoprotein, flavoprotein, ovomacroglobulin, ovostatin, cystatin, avidin, ovalbumin related protein X, ovalbumin related protein Y, and any combination thereof.

48. The method of claim 46 or claim 47, wherein the egg white protein is an ovalbumin (OVA) that comprises the amino acid sequence of a chicken OVA, a goose OVA, a quail OVA, an ostrich OVA, or a duck OVA.

49. The method of any one of claims 46 to 48, wherein the egg white protein has at least 70%, 75%, 80%, 85%, 90%, 95%, 96%, 97%, 98%, or 99% sequence identity to one of SEQ ID NO: 1 to SEQ ID NO: 74.

50. The method of any one of the preceding claims, wherein the recovered protein of interest is suitable for use in a food product.

51. A powdered composition comprising the recovered protein of interest of any one of the preceding claims.

52. A liquid composition comprising the powdered composition of claim 51 and a solvent suitable for animal or human consumption.

53. A method for increasing recovery and purity of a secreted protein of interest, the method comprising steps of:
obtaining recombinant fungal cells capable of expressing a secreted protein of interest;
culturing the recombinant fungal cells under conditions that promote expression and secretion of the recombinant protein of interest into a culturing medium;
centrifuging the culturing medium and excluding the recombinant fungal cells and other cellular components;
microfiltering the centrifuged culturing medium to further remove any residual cell components prior to adding the acid and introducing ammonium sulfate;
adding an acid to the microfiltered culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest; and
recovering the precipitated protein of interest.

54. A method for increasing recovery and purity of a secreted protein of interest, the method comprising steps of:
obtaining recombinant fungal cells capable of expressing a secreted protein of interest;
culturing the recombinant fungal cells under conditions that promote expression and secretion of the recombinant protein of interest into a culturing medium;
centrifuging the culturing medium and excluding the recombinant fungal cells and other cellular components;
microfiltering the centrifuged culturing medium to further remove any residual cell components prior to adding the acid and introducing ammonium sulfate;
adding an acid to the microfiltered culturing medium to reduce the pH to about or below the isoelectric point (pI) of the protein of interest and introducing ammonium sulfate to the culturing medium to achieve an ammonium sulfate concentration above 200 g/l, thereby precipitating the secreted protein of interest;
recovering the precipitated protein of interest;
solubilizing the precipitated secreted protein of interest with water to obtain a solubilized protein of interest;
diafiltering and/or ultrafiltering the solubilized protein of interest;
microfiltering the diafiltered and/or ultrafiltered protein of interested; and
drying the further microfiltered protein of interest, thereby obtaining a dried protein product.

55. The method of any preceding claims, wherein once fermentation is complete, the culturing medium may be diluted and chilled, and, optionally, clarified using a centrifuge.

56. The method of claim 55, wherein the culturing medium is centrifuged and the centrate is filtered to remove any remaining cell debris.

57. The method of claim 56, wherein the filtrate is stored at up to 8° C. for up to 72 hours.

58. The method of any one of the preceding claims, wherein a dialyzed liquid solution is concentrated by 2-fold, 3-fold, 4-fold, 5-fold, 6-fold, 7-fold, 8-fold, 9-fold, or 10-fold relative to the liquid solution before dialysis.

59. The method of any one of the preceding claims, wherein diafiltration comprises a 10 kDa membrane.

60. The method of any one of the preceding claims, wherein a solubilized precipitated secreted protein of interest is diluted to protein concentration target of 40-50 g/L.

61. The method of claim 60, wherein a base, e.g., sodium hydroxide, is added to the solubilized precipitated secreted protein of interest to achieve a pH of about 6 or about 6.5.

62. The method of any one of the preceding claims, wherein before drying, a liquid solution comprising the protein of interest is filtered to capture any microbial contamination.

63. The method of any one of the preceding claims, wherein precipitation of the protein of interest takes from a few hours to almost a day, e.g., 2 hours, 3 hours, 4 hours, 5 hours, 6 hours, 7 hours, 8 hours, 9 hours, 10 hours, 11 hours, 12 hours, 13 hours, 14 hours, 16 hours, 17 hours, 18 hours, 19 hours, or 20 hours.
